(19) United States
(12) Patent Application Publication
(10) Pub. No.: US 2016/0220710 A1
(43) Pub. Date: Aug. 4, 2016

(54) COMPOSITIONS AND METHODS FOR DELIVERING PHARMACEUTICAL AGENTS

(71) Applicant: THE REGENTS OF THE UNIVERSITY OF MICHIGAN, Ann Arbor, MI (US)

(72) Inventors: Rahul K. Keswani, Canton, MI (US); Gi S. Yoon, Ann Arbor, MI (US); Kathleen Stringer, Ann Arbor, MI (US); Gustavo Rosania, Ann Arbor, MI (US); Chao Tian, Ann Arbor, MI (US); Xueding Wang, Canton, MI (US)

(21) Appl. No.: 15/011,047

(22) Filed: Jan. 29, 2016

Related U.S. Application Data
(60) Provisional application No. 62/109,906, filed on Jan. 30, 2015.
(60) Provisional application No. 62/111,921, filed on Feb. 4, 2015.

(51) Int. Cl. A61K 49/22; A61K 31/498; A61K 9/127

(52) U.S. Cl. CPC A61K 49/22; A61K 9/127; A61K 31/498

(57) ABSTRACT

Provided herein are microcrystalline forms of drugs. In particular, provided herein are microcrystalline drug formulations for delivery to macrophages and treatment of disease.

The present application claims priority to U.S. Provisional Patent Application Ser. No. 62/109,906 filed Jan. 30, 2015, and U.S. Provisional Patent Application Ser. No. 62/111,921 filed Feb. 4, 2015, each of which is hereby incorporated by reference in its entirety.

### STATEMENT REGARDING FEDERALLY SPONSORED RESEARCH OR DEVELOPMENT

This invention was supported in part by Grant Nos. R01GM078200 and R01AR060350 awarded by the National Institutes of Health. The government may have certain rights in the invention.

### FIELD OF THE DISCLOSURE

Provided herein are biomimetic microcrystalline forms of drugs. In particular, provided herein are microcrystalline drug formulations for delivery to macrophages and treatment of disease.

### BACKGROUND

The lung is vulnerable to many inflammatory disorders as it is the only internal organ that is exposed constantly to the external environment. Consequently, respiratory diseases cause an immense worldwide health burden. According to the World Health Organization (WHO) and Forum of International Respiratory Societies (FIRS), it is estimated that ˜15% of the world's population suffers from chronic respiratory conditions, including 235 million people suffering from asthma, more than 200 million from chronic obstructive pulmonary disease (COPD) and these numbers are growing every year. In the case of acute respiratory distress syndrome (ARDS), 200,000 Americans per year are affected, with a mortality rate of 40% but no available therapeutic drug available. Healthcare costs for respiratory diseases are an increasing burden on the economies of all countries, but respiratory diseases are rarely on the public health agenda.

Respiratory diseases, such as ARDS, COPD and asthma, are caused by an uncontrolled inflammatory response characterized by dysregulated pro- and anti-inflammatory mediators and increased numbers and/or altered activation of immune cells, including macrophages. Key inflammatory mediators are the pro-inflammatory cytokines tumor necrosis factor α (TNFα) and interleukin-1 (IL-1α and IL-1β), which are required for the initiation and activation of the immune response, and the anti-inflammatory cytokine interleukin-1 receptor antagonist (IL-1RA), which counteracts IL-1 by competitively binding to IL-1 receptor to block signal transduction and resolve inflammation. Continued dysregulation of TNFα and IL-1 expression is a hallmark of chronic inflammatory disorders of the lung. Macrophages play a critical role in the initiation and resolution of lung inflammation, and importantly, one of the major producers of TNFα and IL-1RA.

Existing anti-inflammatory drugs that act by blocking TNFα activity (e.g., etanercept) or by blocking IL-1 via IL-1RA (e.g., anakinra) are currently undergoing preclinical studies or clinical trials as single agents for the potential treatments for COPD, idiopathic pulmonary fibrosis, asthma, and acute respiratory distress syndrome amongst others. However, both TNFα inhibitors and IL-1RA are soluble agents that when systemically injected are poised to affect the whole body indiscriminately and can lead to serious side effects, including increased susceptibility to infection and sepsis.

Accordingly, macrophage-targeted therapeutic strategies that aim to modulate the intracellular signaling pathways affecting TNFα and IL-1RA balance are needed.

### SUMMARY

Provided herein are microcrystalline forms of drugs. In particular, provided herein are microcrystalline drug formulations for delivery to macrophages and treatment of disease.

For example, in some embodiments, the present disclosure provides compositions comprising a biomimetic crystal of a pharmaceutical agent. In some embodiments, the biomimetic crystal is a pure drug crystal or in complex with a counterion or cell-stabilizing agent as a salt, hydrate, solvate or a cocrystal. In some embodiments, the biomimetic crystal has an orthorhombic, triclinic, or monoclinic crystal structure. In some embodiments, the crystal has a needle, cube, blade, prism, or rhomboid habit. In some embodiments, the small molecule pharmaceutical agent is clofazimine. In some embodiments, the orthorhombic crystalline structural form has the configuration space group Pbca. In some embodiments, the salt has the crystal structure shown in the Figures. In some embodiments, the density of the crystals is between 1.25-1.4 g/ml and the crystals have a size of 0.001-20 μm in each dimension. In some embodiments, the salt comprises chloride. In some embodiments, the cell stabilizing agent is hydrochloride. In some embodiments, the salt further comprises one or more additional ions or cell stabilizing agents (e.g. one or more (e.g., two or more) of hydrochloride, hydrobromide, hydroiodide, hydrogen sulfate, sulfate, hydrogen phosphate, phosphate, carbonate, hydrogen carbonate, formate, gluconate, lactate, pyruvate, nitrite, glutarate, tartarate, benzoate, sulfate, fumarate, benzenesulfonate, tosylate, galacturonate, acetate, citrate, nitrate, oxalate, succinate, and maleate). In some embodiments, the ratio of pharmaceutical agent to ion is approximately 0.1 to 2.0. In some embodiments, the composition is provided as a crystal hydrate, crystal salt, or cocrystal. In some embodiments, the composition further comprises a lipid. In some embodiments, the pharmaceutical agent is encapsulated by a liposome comprising the lipid. In some embodiments, the lipid is one or more of phosphatidylcholine, cholesterol, phosphatidylethanolamine, phosphatidylglycerol, phosphatidylinositol, phosphatidylserine, sphingomyelin, cardiolipin, dioleoylphosphatidylglycerol (DOPG), diacylphosphatidylcholine, diacylphosphatidylethanolamine, ceramide, sphingomyelin, cephalin, cholesterol, cerebrosides, diacylglycerols, dioleoylphosphatidylcholine (DOPC), dimyristoylphosphatidylcholine (DMPC), and dioleoylphosphatidylserine (DOPS), diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), di stearoylphosphatidylcholine (DSPC), dipalmitoylphosphatidylcholine (DPPC), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, polyethylene glycol (PEG), or PEG modified lipids. In some embodiments, the lipid or other encapsulating agent is modified to comprise a targeting agent (e.g., including but not limited to, antibodies, mannose, folate, or transferrin). In some embodiments, the composition further comprises one or more of a non-ionic surfactant, a niosome, a polymer, a protein, or a carbohydrate.

Further embodiments provide a method of treating or preventing a disease in a subject, comprising: administering any of the aforementioned compositions to a subject diagnosed with or suspected of having a disease. In some embodiments, the administering reduces or eliminates signs and/or or symptoms of the disease. In some embodiments, the disease is asthma, bronchiolitis, bronchiolitis obliterans, chronic obstructive pulmonary disease (COPD), bronchitis, emphysema, hypersensitivity pneumonitis, idiopathic pulmonary fibrosis, pneumoconiosis, silicosis, meningitis, sepsis, malaria, rheumatoid osteoarthritis, psoriasis, acute respiratory disease syndrome, inflammatory bowel disease, multiple sclerosis, joint inflammation, reactive arthritis, hay fever, atherosclerosis, rheumatoid arthritis, bursitis, gouty arthritis, osteoarthritis, polymyalgia rheumatic arthritis, septic arthritis, infectious arthritis, asthma, autoimmune diseases, chronic inflammation, chronic prostatitis, glomerulonephritis, nephritis, inflammatory bowel diseases, pelvic inflammatory disease, reperfusion injury, transplant rejection, vasculitis, myocarditis, colitis, appendicitis, peptic ulcer, gastric ulcer, duodenal ulcer, peritonitis, pancreatitis, ulcerative colitis, seudomembranous colitis, acute colitis, ischemic colitis, diverticulitis, epiglottitis, achalasia, cholangitis, cholecystitits, hepatitis, Crohn's disease, enteritis, Whipple's disease, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, pneumonitits, pneumoultramicroscopic silicovolcanoconiosis, alvealitis, bronchiolitis, pharyngitis, pleurisy, sinusitis, influenza, respiratory syncytial virus infection, HIV infection, hepatitis B virus infection, hepatitis C virus infection, herpes virus infection disseminated bacteremia, Dengue fever, candidiasis, malaria, filariasis, amebiasis, hydatidcysts, burns, dermatitis, dermatomyositis, sunburn, urticaria, Warts, Wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosis, Goodpasture's syndrome, Behcet's syndrome, allograft rejection, graft-versus-host disease, Type I diabetes, Type II diabetes, ankylosing spondylitis, Berger's disease, Reiter's syndrome, Hodgkin's disease, ileus, hypertension, irritable bowel syndrome, myocardial infarction, sleeplessness, anxiety, local inflammation, or stent thrombosis. In some embodiments, the disease is caused by infection by a microorganism, e.g., Staphylococcus aureus, Streptococcus, Streptococcus pneumonia, Neisseria gonorrhoeae, Mycobacterium tuberculosis, Borrelia burgdorferi, or Haemophilus influenza.

In some embodiments, the composition is targeted to macrophages of the subject (e.g., the composition is phagocytized by the macrophage). In some embodiments, the composition has a biological effect in the subject (e.g., one or more of inhibition of TNFα production, enhancement of IL-1RA production, or downregulation of toll-like receptor expression). In some embodiments, the administering is parentally, via inhalation, or nasally. For example, in some embodiments, the administering is to the lung via inhaler or nebulizer.

Additional embodiments provide the use of any of the aforementioned compositions to treat a disease in a subject.

Other embodiments provide a method of identifying inflammation in a joint of a subject, comprising: a) administering any of the aforementioned compositions or clofazimine to the subject; and b) performing photo-Acoustic Tomography (PAT) of a joint of the subject to identify the presence of the composition in the joint, wherein the presence of the composition in said joint is indicative of inflammation in the joint. In some embodiments, the method further comprises the step of imaging said joint using ultrasound. In some embodiments, the joint is, for example, a knee joint, a finger joint, a toe joint, a hip joint, or an elbow joint. In some embodiments, the presence of inflammation in said joint is indicative of arthritis in said joint. In some embodiments, the composition or clofazimine treats the inflammation in the joint.

Still other embodiments provide the use of any of the aforementioned compositions or clofazimine as a PAT contrast agent for imaging a joint.

Additional embodiments are described herein.

### DESCRIPTION OF THE FIGURES

FIG. 1 shows fluorimetric analysis of (A) CFZ dissolved in DMSO (20 μM) done using conventional solution fluorimetry (bottom—chemical formula of CFZ) and (B) CFZ-TC (bottom—representative picture, scale bar=10 μm) and (C) isolated CLDIs from spleen (bottom—representative picture, scale bar=10 μm) acquired using spectral confocal microscopy. Maximum Fluorescence Yield (MFY) indicates the wavelengths at which maximum fluorescence activity was measured

FIG. 2 shows flow cytometric analysis of RAW264.7 incubated with CLDIs using a variety of laser combinations on a MoFlo Astrios. (top left inset) A single population is observed with RAW264.7 cells alone (shown in grey histogram in each sub-plot). A single population is observed with RAW264.7 cells with CLDIs when excited with UV laser (excitation=355 nm, all emission detector settings), violet laser (excitation=405 nm, all emission detector settings), blue laser (excitation=488 nm, emission=513 nm, 576 nm) and orange laser (excitation=561 nm, emission=579 nm). In all other laser combinations, two populations were observed (labeled 1 and 2). (bottom) Confocal microscopy of RAW264.7 incubated with CLDIs confirmed the phagocytosis of CLDIs using the fluorescence of CLDIs in the Cy5 channel while cells were stained with membrane stain FM 1-43 and nuclear counterstain Hoechst 33342 (Scale bar=10 μm, dotted lines show the visualization planes for the three orthogonal views).

FIG. 3 shows (A) Representative images of brightfield microscopy of sorted cell populations—labeled 1 and 2 in Figure at 488 664/22, 561 692/75 and 640 671/30, scale bar=50 μm. Inset photographs show digitally zoomed-in regions (scale bars=10 μm). Flow cytometric compensation to obtain accurate readout of RAW264.7 cells with CLDIs for viability using (B) propidium iodide (PI) and (C) DAPI.

FIG. 4 shows (A) Flow cytometric analysis of peritoneal exudate in control mice and CFZ-treated mice done using various laser combinations. (B) The populations labeled 1 and 3 at 640 795/70 in (A) were sorted, plated with complete growth media and visualized using standard epifluorescence microscopy. (C) Flow cytometric analysis of peritoneal exudate at various time-points post drug feeding.

FIG. 5 shows (A) Flow cytometry analysis of peritoneal exudate in CFZ mice. (B) The peritoneal exudate was also dual labeled with antibodies for the pan-macrophage marker F4/80 (detected using 405 448/59) followed by either CD86 or CD206 (488 513/26). (C) Cumulative statistics on CLDI(+) cells that are (i) F4/80+, (ii) CD86+ and (iii) CD206+ in CFZ mice at four weeks (4 wk), eight weeks (8 wk) and eight weeks+eight weeks washout (8 wk wo) post drug feeding.

FIG. 6 shows flow cytometric analysis of alveolar exudate at 8 weeks post drug feeding.

FIG. 7 shows a schematic for quantification of peritoneal exudate containing CLDIs.

FIG. 8 shows revisualization of Live CLDI(+) and Live CLDI(−) RAW264.7 cells as a single population in FIG. 3.

FIG. 9 shows normalized Background corrected MFI of cells in relation to the number of CLDIs contained in the cell.

FIG. 10 shows background corrected MFI of cells in relation to the number of CLDIs contained in the cell.

FIG. 11 shows screening for candidate PA imaging contrast agents. (a) Solution absorption spectra of the screened molecules (shown from 300 to 600 nm, at concentrations of 0.2-1 mM) when solubilized in DMSO. (b) Schematic diagram for the setup for PA spectroscopy. OPO: Optical parametric oscillator; BS: beam splitter; BR: black rubber; WT1, WT2: water tanks; T1, T2: transducers; GT: glass tube (c) The solution absorption spectra and PA spectra of CFZ and its protonated forms in solution.

FIG. 12 shows MΦ-Targeting and PA Microscopy of CFZ. (a) Cryosections of spleen and liver in 8-week CFZ-fed mice showed specific and massive accumulation of CFZ in CD68(+) MΦs. (b) PA Microscopy Setup Schematic (WT-Water tank) (c) (left) PA microscopy of the (top) liver and (middle) spleen cryosections of vehicle and CFZ-fed mice and (bottom) RAW264.7 cells alone and incubated with CFZ inclusions (Scale bar=50 μm). (right) Signal Intensity (mV) distribution across various samples (representative data from 3 acquired images).

FIG. 13 shows PA Imaging and Anti-Inflammatory Activity of CFZ. (a) (i) top view, (ii) side view of phantoms loaded with CFZ, CFZ-A, CFZ-HCl or CLDIs (all at 2 mM in DMSO or 2 mM soluble equivalent concentration resuspended in PBS), (iii) Normalised PA signals (in mV) acquired using a dual imaging modality PAT instrument described ahead (b) Anti-inflammatory activity of CFZ as CFZ-A, CFZ-HCl or CLDIs (all at 2 mM in DMSO or 2 mM soluble equivalent concentration resuspended in PBS) in a carrageenan (Car)-based mouse footpad inflammation model (n=4).

FIG. 14 shows clinical Finger-Joint Imaging. (a) Schematic and (b) Photograph of the dual modality US-PA imaging setup (c) Close-up model of the injection site and imaging probe. (d) PA-US (background) images obtained of CFZ-A injected into the human cadaver MCP joint of the index finger.

FIG. 15 shows pigmented Drug Profiling. Identification of pigmented anti-inflammatory drugs via Saturation (S in HSY extracted from RGB of colours photographed on NPC plates), absorption spectrophotometry profile as maximum intensity difference (Iₘₐₓ−Iₘᵢₙ) and wavelength of maximum absorption (λₘₐₓ). Pigmented drugs had high saturation, higher λₘₐₓ (>350 nm) and higher intensity differences.

FIG. 16 shows prediction of CFZ solution forms via ChemAxon. Protonation profile of CFZ under variation of pH in accordance with its pKₐ.

FIG. 17 shows brightfield Imaging of Clofazimine Accumulation. (top, middle) Cryosections of spleen and liver in 8-week CFZ-fed mice imaged using conventional optical microscopy showing bright red inclusions of CFZ. Scale bar=50 μm. (bottom) Spleen isolated CFZ inclusions (CLDIs) present within RAW264.7 MΦs. Scale bar=20 μm.

FIG. 18 shows vehicle liver and spleen immunostaining. Cryosections of spleen and liver in 8-week control-fed mice immunostained using fluorescent markers showing inherent CD68(+) MΦs.

FIG. 19 shows edema (swelling) calculations. Calculation of footpad volume based on measured dimensions.

FIG. 20 shows anti-inflammatory activity of orally-fed CFZ. 8 week CFZ-fed and Control-fed mice (n=4) were injected with carrageenan (Car, 2% in PBS, 30 μl) in the right footpad and with PBS (30 μl) in the left footpad at time t=0 h and the swollen volume (edema) was calculated as shown in FIG. 19.

FIG. 21 shows effect of carrageenan on footpad swelling prior to CFZ injections.

FIG. 22 shows CFZ-HCl injections alone. CFZ-HCl injected feet showed no signs of swelling (p>0.10 at all time-points vs PBS injected).

FIG. 23 shows CLDI injections alone. CLDI injected feet showed no signs of swelling (all p>0.10 vs PBS injected).

FIG. 24 shows CFZ-A and DMSO injections alone. Both CFZ-A and DMSO injected mice showed some swelling (p<0.05, t=24 h vs PBS injected) which was reduced considerably by t=96 h (p>0.1 vs PBS injected). Both CFZ-A and DMSO related swelling were equivalent at all time points (p>0.2 at all time-points, CFZ-A vs DMSO).

FIG. 25 shows background PA and US Images.

FIG. 26 shows purification of CLDIs from the spleen of 8 wk CFZ-fed mice using differential sucrose gradient centrifugation. (A) Brightfield (BF), polarized (diattenuation at 623 nm, slow axis orientation and average transmittance) and fluorescence (Cy5 and FITC) microscopy of spleen CLDIs before (top panel) and after (bottom panel) sucrose gradient centrifugation which shows the two were visually similar. (B) Average Feret max, min and area dimensions of CLDIs before (n=629) and after (n=531) sucrose gradient centrifugation. (C) Size (area) distribution of CLDIs before and after sucrose gradient centrifugation.

FIG. 27 shows phagocytosis of soluble CFZ or CLDIs by RAW 264.7 cells. (A) Brightfield (top panel) and fluorescence (Cy5; lower panel) microscopy of RAW 264.7 cell that were incubated with soluble CFZ or CLDIs for 24 h showing the presence of each CFZ species in the intracellular space. Inset image displays CLDI in vacuole-like intracellular vesicle. Bar=20 μm. (B) Concentration dependent increases in the percentage of CLDI positive cells and the number of CLDIs per cell. Data are the mean (±S.D.) of cell percentage or CLDI number from 5 or 6 images acquired from two different experiments with at least 640 total cells per condition. (C) Soluble CFZ-treated cells displayed increased nuclear MTR signal by 4-fold.

FIG. 28 shows that intracellular CLDIs are not cytotoxic to RAW 264.7 cells. (A) Increasing concentrations of soluble CFZ (◯) lead to decreased cell viability as compared with CLDIs () measured by XTT assay. (B) Mitotracker Red and NucBlue (DAPI) staining of control, soluble CFZ (10 μM) and CLDI (10 μM) treated RAW 264.7 cells shows dissipation of mitochondrial membrane potential in soluble CFZ-treated cells, evidenced by diffuse cytoplasmic Mitotracker staining (middle panel), but not in CLDI treated cells (bottom panel). Bar=10 μm. (C) Quantification of mitochondrial membrane potential dissipation by measurement of nuclear fluorescence intensity of Mitotracker Red in control, CFZ (10 μM) and CLDI (10 μM) treated RAW 264.7 cells. (D) Representative RAW 264.7 cell Western blot of caspase 3, cleaved caspase 3 and cleaved PARP following treatment with increasing concentrations of soluble CFZ or CLDIs for 24 h.

FIG. 29 shows that intracellular CLDIs enhance IL-1RA production and activate Akt in RAW 264.7 cells. (A) IL-1RA production in the media increased in CLDI containing cells compared with negative control cells (DM=DMSO) or cells containing intracellular CFZ. (B) Representative RAW 264.7 cell Western blot of Akt phosphorylated at Ser473 (pAkt) and total Akt detected in the same cell lysates as those assayed for IL-1RA showing up regulation of pAkt with increasing CLDI concentration.

FIG. 30 shows that intracellular CLDIs dampen TLR2 and 4-mediated NF-κB activation and TNFα production in RAW 264.7 cells. (A) TNFα production from non-stimulated cells (naïve) or negative control cells (DM: DMSO or Cont) with or without intracellular soluble (sol.) CFZ or CLDIs following a 6 h stimulation by the TLR2 agonist, Pam3 or the TLR4 agonist, LPS. (B) Representative Western blot of RAW 264.7 cell phosphorylated IκB (p-IκB) and IκB from naïve cells (c) or cells with or without intracellular soluble (sol.) CFZ or CLDIs in response to a 1 h stimulation with Pam3 (top panel) or LPS (bottom panel). (C-E) Representative p65 and isotype IgG (F) immunofluorescence images displaying p65 nuclear translocation in control (C), 4 μM soluble CFZ (D), or 40 μM CLDI (E) treated cells non-stimulated (naïve) or stimulated with Pam3 or LPS for 3 h. Images were used for p65 nuclear:cytoplasmic ratio measurements quantitatively presented in (G). (H) Representative Western blot of RAW 264.7 cell TLR2, TLR4, TLR9 and MyD88 expression in cells with or without intracellular soluble (sol.) CFZ or CLDIs demonstrating the CLDI-induced down regulation of TLR2 and TLR4. (I) Brightfield images displaying mouse primary alveolar macrophages isolated from mice fed with control diet or CFZ for 4 wks (contains soluble CFZ) or 8 wks (contains CLDI), and (J) detection of TLR2 by Western blot shows reduced expression of TLR2 in alveolar macrophages from 8 wk CFZ-fed mice, while 4 wk CFZ-fed mice show increased levels of TLR2 compared to control alveolar macrophages. (* p<0.05, ** p<0.01; ANOVA with Bonferroni's post-hoc tests).

FIG. 31 shows the method used to acquire CLDI area, Feret max and min from brightfield image using ImageJ, and the calculation of volume.

FIG. 32 shows evaluation of CLDI phagocytic activity of RAW 264.7 cells as measured by phagocytic index. Correlation analysis (Pearson's) showed a positive association between CLDI concentration and CLDI phagocytosis (r=0.978, p<0.001).

FIG. 33 A-C show immunofluorescent staining of TLR2, TLR4 and TLR9 in control RAW 264.7 cells or cells treated with soluble CFZ (4 μM) or CLDI (40 μM) for 24 h.

FIG. 34 shows a graphic of CFZ with atomic assignment for ¹H NMR identification and pKₐ values.

FIG. 35 shows (a) Partition ratios of CFZ in spleen, liver, and intestine, in relation to serum and fat tissue at the major sequestration sites at three weeks (3 wk), eight weeks (8 wk) and eight weeks+eight weeks washout (8 wk wo). (b) Bright field optical microscopy revealed red clofazimine CLDIs in (A) spleen and (B) liver tissues at 8 weeks (scale bar=20 μm). (A′-B′) Zooming into relevant regions in A and B. TEM images of fixed (C) spleen and (D) liver cryo-sections revealed the corresponding empty (white) CLDI cavities following extraction of clofazimine during the sample preparation process (scale bar=20 μm). (C′, D′) Zooming into relevant regions of the images shown in C and D.

FIG. 36 shows (a) Representative ¹H NMR spectra of spleen CLDIs, CFZ-A1, CFZ-A2 and CFZ-TC, s—residual solvent peak. (b) Powder X-Ray Diffraction (p-XRD) of spleen CLDIs, CFZ-A1, CFZ-A2 and CFZ-TC showing peaks (crystal diffraction planes) common to CLDIs, CFZ-A1 and CFZ-A2, exclusive to CFZ-TC and in all samples. Inset shows spleen CLDIs p-XRD.

FIG. 37 shows CFZ-HCl crystal structure shown in three orthogonal views to depict the unit cell comprising of 8 CFZ-HCl molecules.

FIG. 38 shows a protonation profile of CFZ under different conditions of pH (simulated in ChemAxon).

FIG. 39 shows microscopic analysis of spleen tissue cryo-sections revealed that CLDIs were completely absent in vehicle animals, while they were present after an eight week CFZ oral diet treatment, and then following an eight week washout period. A-B) Bright field images acquired with the transmitted light microscope revealed red clofazimine CLDIs in tissues at the end of treatment period and following the washout period (scale bar=20 μm). A′-B′) Zooming into relevant regions in A-B; C-D) TEM images of fixed spleen cryo-sections revealed the corresponding empty (white) CLDI cavities following extraction of clofazimine during the sample preparation process (scale bar=20 μm). C′-D′) Zooming into relevant regions of the images shown in C-D revealed the location of CLDIs in the cytoplasm of mononuclear macrophage-like cells, juxtaposed to cell nuclei.

FIG. 40 shows that microscopic analysis of liver tissue cryo-sections revealed the absence of CLDIs in livers of vehicle animals and their presence in liver after an eight week CFZ oral diet treatment, and then following an eight week washout period. A-B) Bright field images acquired with the transmitted light microscope revealed red clofazimine CLDIs in liver at the end of treatment period and then following an eight week washout period (scale bar=20 μm); A′-B′) Zooming into relevant regions in A-B; C-D) TEM images of fixed liver cryo-sections revealed the corresponding empty (white) CLDI cavities following extraction of clofazimine during TEM sample preparation (scale bar=20 μm). C′-D′) Zooming into relevant regions of the images shown in C-D revealed CLDIs localized exclusively in the cytoplasm of Kupffer cells.

FIG. 41 shows deep-etch FFEM of livers of clofazimine treated animals revealed stacked, homogeneous layers of nanocrystalline domains, bounded and embedded in a reticulum of cytoplasmic membranes. A) Surface view of membrane-bound nanocrystalline layers, stacked on top of each other and juxtaposed to a broken CLDI core showing the characteristic cleavage planes (on the left); B) Cross-sectional view of nanocrystalline domains bounded and embedded in a reticulum which represents the zoomed in portion in (A) following sample rotation. C) Nanocrystalline domain of a CLDI reveals orthogonal cleavage planes shown as three orthogonal Cartesian planes. D) & E) Zooming into a membrane surrounding a crystalline domain, viewed perpendicularly to the fracture plane (direction indicated via the three orthogonal Cartesian planes).

FIG. 42 shows clofazimine in DMSO-d6 (CFZ-TC), chemical shifts.

FIG. 43 shows clofazimine in DMSO-d6 (CFZ-TC), observed significant cross-peaks from 2D experiments COSY, NOESY, HMBC.

FIG. 44 shows clofazimine in DMSO-d6 (CFZ-TC), 1H spectra.

FIG. 45 shows clofazimine in DMSO-d6 (CFZ-TC), 13C spectra.

FIG. 46 shows clofazimine in DMSO-d6 (CFZ-TC), HMBC of key cross-peaks helpful towards the chemical shifts assignment on the molecule.

FIG. 47 shows clofazimine in DMSO-d6 (CFZ-TC), NOESY of key cross-peaks helpful towards the chemical shifts assignment on the molecule.

FIG. 48 shows clofazimine in DMSO-d6 (CFZ-TC), NOESY of key cross-peaks helpful towards the chemical shifts assignment on the molecule.

FIG. 49 shows clofazimine in DMSO-d6 (CFZ-TC), COSY (right side of the spectra) of key cross-peaks helpful towards the chemical shifts assignment on the molecule.

FIG. 50 shows clofazimine in DMSO-d6 (CFZ-TC), HSQC (left side of the spectra).

FIG. 51 shows clofazimine in DMSO-d6 (CFZ-TC), HSQC (right side of the spectra).

FIG. 52 shows HSQC Spectra in DMSO-d6 for (a) Spleen CLDIs, (b) CFZ-A1 and (c) CFZ-A2. CFZ-A1, CFZ-A2 and Spleen CLDIs have identical HSQC spectra. (X-axis—1H ppm, 1H NMR Spectra overlay on top horizontal line, Y-axis—13C ppm).

FIG. 53 shows that nanoSIMS depth profile plotting integrated counts at each sputtering depth using normalized depth. Various elemental ratios are plotted (a) C/Cl, (b), N/Cl, (c) P/Cl, (d) S/Cl, (e) Si/Cl and (f) O/Cl indicative of higher than expected chlorine presence in the CLDI samples. X-axis, Normalized depth (y/d) where d=total sputtering depth. Y-axis, Normalized atomic ratios

FIG. 54 shows scanning electron microscopy and measured cross-sectional elemental profiles (C, N, Cl, O, S, P, Si, F) of (a,b) CFZ-HCl, (c,d) spleen CLDIs (a,c) before primary ion beam sputtering and (b,d) after primary ion beam sputtering on a silicon wafer

FIG. 55 shows a comparison between CFZ-TC and the closest crystal analog identified in CCDC (refcode: DAKXUI01) which has a triclinic crystal packing pattern.

FIG. 56 shows representative Raman spectra of CLDIs, CFZ-HCl and CFZ-TC using a 785 nm laser.

FIG. 57 shows a comparison between predicted and absolute CFZ-HCl p-XRD pattern based upon its orthorhombic lattice configuration.

FIG. 58 shows that (a) CFZ precipitates under different NH4Cl (1000, 100, 10 and 0 mM) and HCl (10, 1, 0 μM) concentrations, (b) pH of reactive agent used to precipitate CFZ, (c) pH of supernatant following the formation of precipitates. (b,c) X-axis—HCl concentration. Each data series shows different concentration of NH4Cl.

FIG. 59 shows a solubility profile of CFZ in HCl—Representative plot of one of three replicates. Y-axis shows (%) precipitate vs solubilized fraction. X-axis shows the molarity of HCl used.

FIG. 60 shows a solubility profile of CFZ in NH4Cl—Representative plot of one of three replicates. Y-axis shows (%) precipitate vs solubilized fraction. X-axis shows the molarity of NH4Cl used.

FIG. 61 shows clofazimine (CFZ) bioaccumulation and crystal formation in the liver occurs after 2 weeks. (A) Representative brightfield and fluorescence (FITC for CFZ and Cy5 for CFZ-HCl) images of liver sections from control mice and mice treated with CFZ for 2, 4 and 8 weeks. These histological findings are corroborated by the (B) fluorescence intensity values (FITC and Cy5 channels) of liver sections from control and CFZ-treated mice for which the Cy5 signal increased over time. (C) F4/80 (DAB) immunohistochemistry of a representative liver section from an 8 week CFZ-treated mouse in which CFZ crystals (Cy5) are sequestered inside F4/80 positive macrophages. CV, central vein. Scale bar=100 μm.

FIG. 62 shows that clofazimine (CFZ) bioaccumulation reduces caspase 1 (Casp 1) and IL-1β cleavage levels in the liver, but increases IL-1RA expression. (A) Western blots of liver homogenates showing that Casp 1 and IL-1β cleavage was not altered by 2 weeks of CFZ treatment, but 8 weeks of treatment reduced the detection of Casp 1 and IL-1β cleavage. (B) Densitometry of cleaved Casp 1 and IL-1β protein in 2 and 8 week-treated liver blots, normalized to actin (n=4). (C) Levels of TNFα in the spleen, lungs and kidneys were unchanged by CFZ treatment but there was a significant decrease in liver samples from 2 week CFZ-treated mice that was not evident at 8 weeks (n=4-5). (D) The organs that bioaccumulate CFZ crystals—liver, spleen and lung—displayed increased IL-1RA expression while the kidney, which does not accumulate CFZ, did not show changes in IL-1RA expression (n=4-5). (E) Serum IL-1RA levels were unchanged following 2 weeks but were increased following 8 weeks of CFZ treatment.

FIG. 63 shows systemic CFZ bioaccumulation and crystal formation dampens carrageenan-induced inflammatory response in the mouse footpad. (A) Representative photographs of paws 48 h post-carageenan (CAR) or PBS footpad injection showing markedly reduced paw swelling in 8 week CFZ-treated mice compared with control mice. (B) Paw swelling, as measured by foot volume, was reduced, on average by 57% as early as 4 h post-carageenan injection in 8 week CFZ-treated mice compared with control mice. This reduction was sustained over the 48 h study. Footpad homogenate cytokine levels IL-1β (C) and TNFα (D) were dampened in 8 week CFZ-treated mice at 48 h compared with untreated and injured paws. (E) Representative photographs of paws 48 h post-CAR or PBS footpad injection in 2 week CFZ-treated or control mice, which (F) resulted in only a minor reduction in swelling at 4 and 48 h. Associated footpad homogenate (G) TNFα and (H) IL-1β levels were unchanged. PBS-injected paws did not show any difference in cytokine expression between CFZ-treated or control paws. n=5-6/experiment.

FIG. 64 shows that dampened acute inflammatory response in foot is mediated by circulating IL-1RA. (A) 2 week CFZ-treated and control mice display similar serum IL-1RA levels before (0 h) and after (48) carrageenan injection. (B) Serum IL-1RA concentrations are significantly increased after 8 weeks of CFZ treatment compared to control mice (0 h). (C) Representative images of inflamed paws of 6 week CFZ-treated wild-type (WT) and IL-1RA KO mice 48 h after carrageenan injection. (D) Paw swelling, as measured by foot volume, was reduced at 4 h and 48 h post-carageenan injection, respectively, in 6 week CFZ-treated WT mice compared with control WT mice. (E) Footpad homogenate cytokine levels IL-1β were dampened in 6 week CFZ-treated mice at 48 h compared with control and injured paws (top left panel). IL-1RA KO mice displayed radically increased IL-1β levels in both untreated and CFZ-treated mice with carrageenan injection compared to WT mice (E, top panel). TNFα expression was higher in IL-1RA KO mice compared to WT mice (E, bottom panel).

FIG. 65 shows that CFZ bioaccumulation enhances resistance to acute lung injury and improves mouse survival. (A). Control mice lung function progressively deteriorated at a higher rate than CFZ-treated mice as CFZ-treated mice displayed higher arterial oxygen levels at day 5 and day 6 (B). Image of lung injury in control and CFZ-treated mice showed reduced injured area caused by LPS. (C) Image of lung injury in control and CFZ-treated mice showed reduced injured area caused by LPS (D) Albumin levels after LPS. (E) TNF-α and (F) IL-1β expression after LPS.

FIG. 66 shows Clofazimine (CFZ) bioaccumulation and crystal formation in the lung occurs after 2 weeks in CD68(+) macrophages. Representative brightfield and fluorescence (FITC for CD68 and Cy5 for CFZ) images of lung sections from control mice and mice treated with CFZ for 2 and 8 weeks. Scale bar=10 μm.

FIG. 67 shows Clofazimine (CFZ) bioaccumulation does not affect IL-1β cleavage in the spleen, lung or kidney.

FIG. 68 shows (A) 6 week CFZ-treated IL-1RA knockout (KO) mice show reduced body weight compared to control diet treated littermates and wildtype (WT) mice. (B) IL-1RA KO mice display reduced liver weight compared to WT, but spleen are comparable. (C) Brightfield images of live alveolar macrophages isolated from WT and IL-1RA KO mice after 6 week control diet or CFZ treatment. CFZ crystals could be seen in both WT and IL-1RA KO macrophages after CFZ treatment.

FIG. 69 shows Daily monitoring of changes in heart rate (A), breath weight (B) and pulse distention (C) in 8 week CFZ-treated and control mice after PBS (white circle) or LPS (grey circle)-induced acute lung injury.

### DEFINITIONS

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human or non-human mammal subject.

As used herein, the term “diagnosed,” as used herein, refers to the recognition of a disease by its signs and symptoms (e.g., resistance to conventional therapies), or genetic analysis, pathological analysis, histological analysis, and the like.

As used herein, the term “effective amount” refers to the amount of a compound (e.g., a compound of the present disclosure) sufficient to effect beneficial or desired results. An effective amount can be administered in one or more administrations, applications or dosages and is not limited to a particular formulation or administration route.

As used herein, the term “co-administration” refers to the administration of at least two agent(s) (e.g., a compound of the present disclosure) or therapies to a subject. In some embodiments, the co-administration of two or more agents/therapies is concurrent. In some embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

As used herein, the term “pharmaceutical composition” refers to the combination of an active agent with a carrier, inert or active, making the composition especially suitable for diagnostic or therapeutic use in vivo, in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa., (1975)).

As used herein, the term “cell stabilizing agent” refers to an agent (e.g., ion, lipid, or other agent) in complex with a biocrystalline mimetic of pharmaceutical agent. In some embodiments, the cell stabilizing agent stabilizes the complex in vivo.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water and industrial samples. Such examples are not however to be construed as limiting the sample types applicable to the present disclosure.

As used herein, the terms “purified” or “to purify” refer, to the removal of undesired components from a sample. As used herein, the term “substantially purified” refers to molecules that are at least 60% free, at least 65% free, at least 70% free, at least 75% free, at least 80% free, at least 85% free, at least 90% free, at least 95% free, at least 96% free, at least 97% free, at least 98% free, at least 99% free, or 100% free from other components with which they usually associated.

As used herein, the term “modulate” refers to the activity of a compound (e.g., a compound of the present disclosure) to affect (e.g., to promote or retard) an aspect of cellular function.

As used herein, the phrase “in need thereof” means that the subject has been identified as having a need for the particular method or treatment. In some embodiments, the identification can be by any means of diagnosis. In any of the methods and treatments described herein, the subject can be in need thereof. In some embodiments, the subject is in an environment or will be traveling to an environment in which a particular disease, disorder, condition, or injury is prevalent.

### DETAILED DESCRIPTION

Provided herein are microcrystalline forms of drugs. In particular, provided herein are microcrystalline drug formulations for delivery to macrophages and treatment of disease.

The elucidation of molecular mechanisms influencing the solubility of poorly soluble chemical agents in different cells, tissues and organs of mammals is interesting from a fundamental chemical and biological perspective. Cells are able to eliminate soluble chemical agents via metabolism and facilitated or active transport across the plasma membrane. Moreover, in the case of foreign, insoluble particles, phagocytic cells of the immune system are especially equipped to actively ingest these particles and isolate them from the rest of the organism. However, for poorly soluble compounds, that can exist both as soluble and insoluble forms within the cells of an organism, the mechanisms controlling the bioaccumulation and distribution of soluble and insoluble forms of these agents in the different cells and organs of the body are not known.

Many FDA-approved drugs (e.g., clofazimine, amiodarone, azithromycin, chloroquine, gefitinib) fall within the class of poorly soluble compounds that are actively sequestered within macrophages (Ohkuma, et al., J. Cell Biol. 1981, 90, 656-664; Poole, et al., J. Cell Biol. 1981, 90, 665-669; Maxfield, et al., J. Cell Biol. 1982, 95, 676-681; Quaglino, et al., Am. J. Physiol. Lung Cell. Mol. Physiol. 2004, 287, 438-447; Bergman, et al., Int. J. Pharm. 2007, 341, 134-142; Gladue, et al., Antimicrob. Agents Chemother. 1989, 33, 277-282.). Elucidating the mechanisms affecting their solubility and accumulation inside cells is relevant to understanding drug toxicity, disposition and efficacy (Fu, et al., Nat. Chem. 2014, 6, 614-622). Particularly, weakly basic molecules have been implicated to be accumulated via the lysosomal pathway in macrophages due to the decrease in pH of the vesicles that contain the drug (Ohkuma et al., supra). Research into mechanisms responsible for in vivo drug bioaccumulation and retention has been very sparse.

CFZ (Harbeck, et al., Ann. Pharmacother. 1999, 33, 250; Levy, L. Am. J. Trop. Med. Hyg. 1974, 23, 1097-1109; Aplin, et al., Experientia 1975, 31, 468-469; McDougall, et al., Br. J. Dermatol. 1980, 102, 227-230; McDougall, Int J Lepr. Other Mycobact. Dis. 1974, 42, 1-12) is an FDA-approved riminophenazine antibiotic that has been remarkably effective against mycobacterial infections such as leprosy (Tolentino, et al., Int J Lepr. Other Mycobact. Dis. 1974, 42, 416-418; Karuru, et al. Lepr. Rev. 1970, 41, 83-88; Leiker, et al., Lepr. Rev. 1971, 42, 125-130) and mycobacterium avium infections in AIDS patients (Rensburg, et al., Antimicrob. Agents Chemother. 1992, 36, 2729-2735; Reddy, et al., J. Antimicrob. Chemother. 1999, 43, 615-623). It has also attracted attention because of its anti-inflammatory (Cholo, et al., J. Antimicrob. Chemother. 2012, 67, 290-298; Helmy, et al., Lepr. Rev. 1972, 42, 162-177) and immunomodulatory (Ren, et al., PLoS One 2008, 3, e4009) properties, and it is especially used for treating leprotic skin inflammations (erythema leprosum nodosum) (Cholo et al., supra; Helmy et al., supra; Barry, et al., Lepr. Rev. 1965, 36, 3-7; Barry, et al., Nature 1957, 179, 1013-1015).

The chemical structure of CFZ (3-(p-chloroanilino)-10-(p-chlorophenyl)-2, 10-dihydro-2-isopropyliminophenazine) is

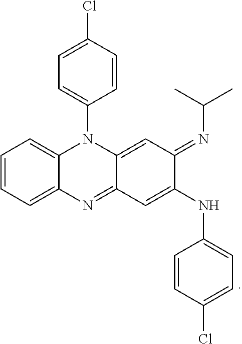 [SMILES: CC(C)/N=c1\cc2n(-c3ccc(Cl)cc3)c3ccccc3nc-2cc1Nc1ccc(Cl)cc1]

Derivatives of CFZ are described, for example, in Franzblau et al., Antimicrob Agents Chemother 1988; 32:1583-5; Jagannath C, et al., Am J Respir Crit Care Med 1995; 151:1083-6; O'Sullivan J F, et al., Health Cooperation Papers 1992; 12:191-7; and O'Connor R, et al., J Chromatogr B Biomed Appl 1996; 681:307-15; each of which is herein incorporated by reference in its entirety.

Accordingly, provided herein are compositions and method for the synthesis, characterization and composition of biomimetic, anti-inflammatory macrophage-targeted, microcrystalline drug formulations (e.g., of CFZ). Alveolar macrophages are primary sites for the anti-inflammatory response in the lung. During or after the inflammation, biomimetic drug crystal formulations are inhaled by the patient or can be administered via intravenous/systemic injections/oral route leading to active uptake by macrophages through phagocytosis. This targeted accumulation causes the macrophages to become more efficient at their anti-inflammatory activity via both down-regulation of TNFα as well as up-regulation of IL-1RA. Both acute as well as chronic disorders are targeted through this approach. Since these are stable drug formulations, their effect is sustained for a long-period of time without the need for repeated dosages. Moreover, since they are targeted to macrophages, their effect is localized to the site of inflammation (e.g., lung or joints), thereby reducing any systemic drug side effects.

### I. Compositions

Provided herein are biomimetic drug microcrystals of small molecule pharmaceutical agents (e.g., CFZ). In some embodiments, the agents are present as salts comprising counterions. In some embodiments, compositions mimic the natural solid state in terms of the drug protonation state, counterions, salt form or crystal polymorph of the drugs (e.g., a biomimetic of a naturally occurring crystal form present in the CLDIs that accumulate in macrophages of drug-treated animals upon long term oral dosing).

The present disclosure is illustrated with CFZ formulations. However, the present disclosure is not limited to CFZ. Crystalline forms of other agents (e.g., agents that accumulate as CLDIs in vivo) are specifically contemplated by the present disclosure.

In some embodiments, active agents are present as a salt with a counterion. For example, in some embodiments, CFZ is present as a salt is positively charged and formulations comprise one or more anions. In some embodiments, the anion is chloride. In some embodiments, the anion is one or more of bromide, iodide, sulfate, phosphate, carbonate, acetate, citrate, nitrate, oxalate, succinate, or maleate. In some embodiments, the ratio of drug:counterion is between approximately 0.1-2.0, although other ratios find use in some embodiments.

Crystalline forms of active agents are formed using any suitable method. For example, in some embodiments, the active agent is dissolved in a solvent (e.g., methanol) and equal volumes of anti-solvents (e.g., comprising counterion) are added to obtain drug crystals. The supernatant is then removed, the crystals are washed, and optionally lyophilized. In some embodiments, crystals are generated by mechanical milling and homogenization.

In some embodiments, crystalline formulations exhibit specific dichroism properties. The interaction of CLDIs with polarized light is highly distinct from other pure CFZ crystals. Such interactions can be quantitatively measured using linear dichroism, an optical parameter that measures optical anisotropy within solid structures of transmittance of light. In some embodiments, dichroic ratios of the formulations described herein at 546 nm and 623 nm (LD623/LD546) is approximately 0.5±0.1. In some embodiments, crystals have an orthorhombic crystal structure (space group: Pbca) with a denser molecular packing compared to triclinic crystals. In some embodiments, crystalline formulations of CFZ described herein have a bulk density in the range of 1.33-1.4 g/ml. In some embodiments, crystalline formulations of CFZ described have a size range between 0.001-20 μm in one and/or two dimensions.

In some embodiments, compositions further comprise one or more lipids. In some embodiments, the lipids are present as a liposome that encapsulates the pharmaceutical agent (e.g., to mimic cellular membranes). For example, in some embodiments, biomimetic forms of the crystal are formed using a remote loading of drugs via ammonium salt method or direct lipid encapsulation of ammonium salt precipitated crystal salt of drug (See e.g., Ceh et al., Langmuir, 1995, 11 (9), pp 3356-3368).

In some embodiments, compositions are provides as crystal hydrates, crystal solvates, or cocrystals.

In some embodiments, the pharmaceutical agent is encapsulated in a niosome (See e.g., Moghassemi et al, Journal of Controlled Release, Volume 185, 10 Jul. 2014, Pages 22-36). Niosomes are a class of molecular cluster formed by self-association of non-ionic surfactants in an aqueous phase.

In some embodiments, the lipid (e.g., phospholipid) structures surrounding crystalline pharmaceutical agent is tailored to the target organ/tissue lipid composition. For example, the natural lipid composition in the lungs is rich in choline lipids. Hence, in some embodiments, synthetic lipids comprising phosphatidylcholines are used in formulating compositions for delivery to the lung. The specific composition and morphology of the formulation is modulated through temperature and concentrations of lipid as well as drug:lipid ratio (See e.g., Keswani et al., Mol Pharm. 2013 May 6; 10(5):1725-35; herein incorporated by reference in its entirety).

In some embodiments, lipids are functionalized to aid in phagocytosis by macrophages. For example, in some embodiments, compositions are enveloped with mannose-conjugated phospholipids that are internalized via the CD206/CD205 receptor on macrophages.

In some embodiments, at least one (or some) of the lipids is/are amphipathic lipids, defined as having a hydrophilic and a hydrophobic portion (typically a hydrophilic head and a hydrophobic tail). The hydrophobic portion typically orients into a hydrophobic phase (e.g., within the bilayer), while the hydrophilic portion typically orients toward the aqueous phase (e.g., outside the bilayer, and possibly between adjacent apposed bilayer surfaces). The hydrophilic portion may comprise polar or charged groups such as carbohydrates, phosphate, carboxylic, sulfato, amino, sulfhydryl, nitro, hydroxy and other like groups. The hydrophobic portion may comprise apolar groups that include without limitation long chain saturated and unsaturated aliphatic hydrocarbon groups and groups substituted by one or more aromatic, cyclo-aliphatic or heterocyclic group(s). Examples of amphipathic compounds include, but are not limited to, phospholipids, aminolipids and sphingolipids.

In some embodiments, the lipids are phospholipids. Phospholipids include without limitation phosphatidylcholine, phosphatidylethanolamine, phosphatidylglycerol, phosphatidylinositol, phosphatidylserine, and the like. It is to be understood that other lipid membrane components, such as cholesterol, sphingomyelin, cardiolipin, etc. may be used.

The lipids may be anionic and neutral (including zwitterionic and polar) lipids including anionic and neutral phospholipids. Neutral lipids exist in an uncharged or neutral zwitterionic form at a selected pH. At physiological pH, such lipids include, for example, dioleoylphosphatidylglycerol (DOPG), diacylphosphatidylcholine, diacylphosphatidylethanolamine, ceramide, sphingomyelin, cephalin, cholesterol, cerebrosides and diacylglycerols. Examples of zwitterionic lipids include without limitation dioleoylphosphatidylcholine (DOPC), dimyristoylphosphatidylcholine (DMPC), and dioleoylphosphatidylserine (DOPS). An anionic lipid is a lipid that is negatively charged at physiological pH. These lipids include without limitation phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

Collectively, anionic and neutral lipids are referred to herein as non-cationic lipids. Such lipids may contain phosphorus but they are not so limited. Examples of non-cationic lipids include lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), di stearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, and cholesterol.

Additional nonphosphorous containing lipids include stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide and the like, diacylphosphatidylcholine, diacylphosphatidylethanolamine, ceramide, sphingomyelin, cephalin, and cerebrosides. Lipids such as lysophosphatidylcholine and lysophosphatidylethanolamine may be used in some instances. Noncationic lipids also include polyethylene glycol-based polymers such as PEG 2000, PEG 5000 and polyethylene glycol conjugated to phospholipids or to ceramides (referred to as PEG-Cer).

In some embodiments, lipids are cationic lipids (e.g., those described herein).

In some instances, modified forms of lipids may be used including forms modified with detectable labels such as fluorophores. In some instances, the lipid is a lipid analog that emits signal (e.g., a fluorescent signal). Examples include without limitation 1,1′-dioctadecyl-3,3,3′,3′-tetramethylindotricarbocyanine iodide (DiR) and 1,1′-dioctadecyl-3,3,3′,3′-tetramethylindodicarbocyanine (DiD).

In some embodiments, the lipids are biodegradable in order to allow release of encapsulated agent in vivo and/or in vitro. Biodegradable lipids include but are not limited to 1,2-dioleoyl-sn-glycero-3-phosphocholine (dioleoyl-phosphocholine, DOPC), anionic 1,2-di-(9Z-octadecenoyl)-sn-glycero-3-phospho-(1′-rac-glycerol) (dioleoyl-phosphoglycerol, DOPG), and 1,2-distearoyl-sn-glycero-3-phosphoethanolamine (distearoyl-phosphoethanolamine, DSPE). Non-lipid membrane components such as cholesterol may also be incorporated.

One or more of the lipids may be functionalized lipids. In some embodiments, the reactive group is one that will react with a crosslinker (or other moiety) to form crosslinks between such functionalized lipids. The reactive group may be located anywhere on the lipid that allows it to contact a crosslinker and be crosslinked to another lipid in an adjacent apposed bilayer. In some embodiments, it is in the head group of the lipid, including for example a phospholipid. An example of a reactive group is a maleimide group. Maleimide groups may be crosslinked to each other in the presence of dithiol crosslinkers such as but not limited to dithiolthrietol (DTT). An example of a functionalized lipid is 1,2-dioleoyl-sn-glycero-3-phosphoethanolamine-N-[4-(p-maleimidophenyl) butyramide, referred to herein as MPB. Another example of a functionalized lipid is 1,2-distearoyl-sn-glycero-3-phosphoethanolamine-N-[maleimide(polyethylene glycol)2000] (also referred to as maleimide-PEG 2k-PE). Another example of a functionalized lipid is dioleoyl-phosphatidylethanolamine 4-(N-maleimidomethyl)-cyclohexane-1-carboxylate (DOPE-mal).

It is to be understood that the disclosure contemplates the use of other functionalized lipids, other functionalized lipid bilayer components, other reactive groups, and other crosslinkers. In addition to the maleimide groups, other examples of reactive groups include but are not limited to other thiol reactive groups, amino groups such as primary and secondary amines, carboxyl groups, hydroxyl groups, aldehyde groups, alkyne groups, azide groups, carbonyls, haloacetyl (e.g., iodoacetyl) groups, imidoester groups, N-hydroxysuccinimide esters, sulfhydryl groups, pyridyl disulfide groups, and the like.

Functionalized and non-functionalized lipids are available from a number of commercial sources including Avanti Polar Lipids (Alabaster, Ala.).

In some embodiments, compositions further comprise one or more additional agents. Examples include, but are not limited to, polymers, proteins, carbohydrates, or other natural or artificial molecular components that serve to enhance the targeting or activity of the active agent (e.g., by promoting the binding to or phagocytosis by alveolar macrophages, or by slowing down the degradation/decomposition/clearance by macrophages in other sites of the body).

### II. Treatment Methods

Embodiments of the present disclosure provide methods of using the aforementioned crystalline drug formulations (e.g., CFZ) in the treatment of disease (e.g., respiratory or inflammatory disease). The present disclosure is not limited to particular inflammatory diseases. Exemplary diseases are described herein.

The compositions described herein find use in the treatment of a variety of acute and chronic respiratory disease. Examples include, but are not limited to, asthma, bronchiolitis, bronchiolitis obliterans, chronic obstructive pulmonary disease (COPD), bronchitis, emphysema, hypersensitivity pneumonitis, idiopathic pulmonary fibrosis, pneumoconiosis, or silicosis.

Further example of inflammatory disease include, but are not limited to acute bacterial or viral infection e.g. meningitis, sepsis, malaria or chronic inflammatory diseases such as rheumatoid osteoarthritis, psoriasis, acute respiratory disease syndrome, inflammatory bowel disease (ulcerative colitis and Crohn's disease), multiple sclerosis, etc. In some embodiments, the inflammatory disease is local inflammation (e.g., at local sites such as eyes/cornea/conjunctiva, sclera, vitreous humor etc.).

In some embodiments, the compositions described herein find use in the treatment of joint inflammation (either acute or chronic) induced due to infection of any other organs via the causative microorganisms. These conditions can also be categorized as septic arthritis or infectious arthritis or inflammatory arthritis. In some embodiments, infectious arthritis is caused by Staphylococcus aureus, Streptococcus, Streptococcus pneumonia, Neisseria gonorrhoeae, Mycobacterium tuberculosis, Borrelia burgdorferi, or Haemophilus influenza.

The compositions further find use in the treatment of arthritis. Arthritis also develops in people who have infections that do not involve the bones or joints, such as infections of the genital organs or digestive organs or ocular regions. This type of arthritis is a reaction to that infection and so is called reactive arthritis. In reactive arthritis, the joint is inflamed but not actually infected.

Additional types of inflammatory disorders that may be treated as described herein include a variety of disease states, including diseases such as hay fever, atherosclerosis, arthritis (rheumatoid, bursitis, gouty arthritis, osteoarthritis, polymyalgia rheumatic, etc.), asthma, autoimmune diseases, chronic inflammation, chronic prostatitis, glomerulonephritis, nephritis, inflammatory bowel diseases, pelvic inflammatory disease, reperfusion injury, transplant rejection, vasculitis, myocarditis, colitis, appendicitis, peptic ulcer, gastric ulcer, duodenal ulcer, peritonitis, pancreatitis, ulcerative colitis, seudomembranous colitis, acute colitis, ischemic colitis, diverticulitis, epiglottitis, achalasia, cholangitis, cholecystitits, hepatitis, Crohn's disease, enteritis, Whipple's disease, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, pneumonitits, pneumoultramicroscopic silicovolcanoconiosis, alvealitis, bronchiolitis, pharyngitis, pleurisy, sinusitis, influenza, respiratory syncytial virus infection, HIV infection, hepatitis B virus infection, hepatitis C virus infection, herpes virus infection disseminated bacteremia, Dengue fever, candidiasis, malaria, filariasis, amebiasis, hydatidcysts, burns, dermatitis, dermatomyositis, sunburn, urticaria, Warts, Wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosis, Goodpasture's syndrome, Behcet's syndrome, allograft rejection, graft-versus-host disease, Type I diabetes, Type II diabetes, ankylosing spondylitis, Berger's disease, Reiter's syndrome, Hodgkin's disease, ileus, hypertension, irritable boWel syndrome, myocardial infarction, sleeplessness, anxiety and stent thrombosis.

The compositions described herein provide a variety of advantages over the delivery of existing formulations of CFZ. Examples include, but are not limited to, enhanced anti-inflammatory response compared to soluble formulations, dual cell-targeted anti-inflammatory response by increasing endogeneous IL-1RA production and reduction in TNF-α levels, enhanced and specific delivery to alveolar macrophages, reduced/No toxicity, and sustained controlled release delivery of therapeutics, thus not requiring repeated medication resulting in better quality of care and patient adherence.

In some embodiments, administration of microcrystalline formulations of drugs (e.g., CFZ) that target macrophages results in several molecular effects. In some embodiments, one or more of the following physiological effects is observed following administration. Inhibition of TNFα production: The active phagocytosis and intracellular compartmentalization of CLDIs by macrophages results in the dampening of NF-κB activation and TNFα release in response to TLR2 and TLR4 agonists. In addition, enhancement of IL-1RA production is also a result of the formulations described herein. The phagocytosis of CLDIs augments the activation of PI-3K/Akt signaling pathways, which leads to enhanced IL-1RA production and release. Downregulation of Toll-like receptors (TLR): The active phagocytosis of CLDIs by macrophages results in the specific downregulation of cell surface TLR2 and TLR4, which play important roles in modulating immune responses.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation, insufflation or by a transdermal patch. Transdermal patches dispense a drug at a controlled rate by presenting the drug for absorption in an efficient manner with minimal degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The compounds described herein, optionally together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

The dose when using the compounds and formulations described herein can vary within wide limits and as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis is conducted or on whether further active compounds are administered in addition to the compounds. Representative doses include, but not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example 2, 3 or 4 doses. Depending on the individual and as deemed appropriate from the patient's physician or caregiver it may be necessary to deviate upward or downward from the doses described herein.

The amount of active ingredient, or an active salt or derivative thereof, for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated or prophylaxis is conducted or on whether further active compounds are administered in addition to the compounds described herein and as part of a drug combination. The dosage regimen for treating a disease condition with the compounds and/or compositions is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods described herein.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4 part administrations. If appropriate, depending on individual behavior, it may be necessary to deviate upward or downward from the daily dose indicated.

The compounds can be administrated in a wide variety of oral and parenteral dosage forms. It will be obvious to those skilled in the art that the following dosage forms may comprise, as the active component, either a compound described herein or a pharmaceutically acceptable salt, solvate or hydrate of a compound described herein.

For preparing pharmaceutical compositions, the selection of a suitable pharmaceutically acceptable carrier can be either solid, liquid or a mixture of both. Solid form preparations include powders, tablets, pills, capsules, cachets, suppositories and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desire shape and size. The powders and tablets may contain varying percentage amounts of the active compound. A representative amount in a powder or tablet may contain from 0.5 to about 90 percent of the active compound; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed, as a solvent or suspending medium. For this purpose any bland fixed oil may be employed including synthetic mono- or diglycerides. In addition, fatty acids such as oleic acid find use in the preparation of injectables.

The compounds according may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous formulations suitable for oral use can be prepared by dissolving or suspending the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well-known suspending agents.

Also included are solid form preparations which are intended to be converted, shortly before use, to liquid form preparations for oral administration. Such liquid forms include solutions, suspensions and emulsions. These preparations may contain, in addition to the active component, colorants, flavors, stabilizers, buffers, artificial and natural sweeteners, dispersants, thickeners, solubilizing agents and the like.

For topical administration to the epidermis the compounds may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compounds or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds as an aerosol can be prepared by processes well known to the person skilled in the art. For their preparation, for example, solutions or dispersions of the compounds in water, water/alcohol mixtures or suitable saline solutions can be employed using customary additives, for example benzyl alcohol or other suitable preservatives, absorption enhancers for increasing the bioavailability, solubilizers, dispersants and others and, if appropriate, customary propellants, for example include carbon dioxide, CFCs, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 50 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

The compounds may optionally exist as pharmaceutically acceptable salts including pharmaceutically acceptable acid addition salts prepared from pharmaceutically acceptable nontoxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. Certain pharmaceutically acceptable salts are listed in Berge, et al., Journal of Pharmaceutical Sciences, 66:1-19 (1977), incorporated herein by reference in its entirety.

### III. Imaging

Embodiments of the present disclosure provide compositions and methods for imaging. In some embodiments, the compositions are used as imaging agents in photoacoustic tomography (PAT). Photoacoustic (PA) detection relies on intrinsic absorption at specific excitation laser-generated wavelengths resulting in ultrasonic waves detected via conventional acoustic transducers. For imaging applications, longer wavelengths are advantageous because they afford greater imaging depth, with reduced potential for phototoxicity. Experiments conducted herein demonstrated that clofazimine yielded optimal PAT signals at 450 to 540 nm. Further experiments demonstrated that clofazimine accumulates in macrophages and thus provides a marker for inflammation.

Accordingly, in some embodiments, provided herein is the use of clofazimine or crystals comprising clofazimine as described herein as PAT imaging or contrast agents to identify inflammation. In some embodiments, PAT is used in combination with ultrasound imaging.

In some embodiments, PAT imaging with clofazimine contrast agents is utilizing to identify inflammation in a joint (e.g., a knee joint, a finger joint, a toe joint, a hip joint, and an elbow joint). In some embodiments, the inflammation is associated with arthritis in the joint. Thus, in some embodiments, the presence of a PAT signal associated with clofazimine in a joint is indicative of a diagnosis of arthritis or other inflammation in the joint.

In some embodiments, clofazimine and crystals thereof further find use in the treatment of inflammation in a joint (e.g., as described herein).

### EXPERIMENTAL

### Example 1

### Flow Cytometry and Image-Based Functional Studies

### Materials and Methods

### Animal Experiments.

Mice (4 week old, male C57Bl6) were purchased from the Jackson Laboratory (Bar Harbor, Me.) and acclimatized for 1 week in a specific-pathogen-free animal facility. Animal care was provided by the University of Michigan's Unit for Laboratory Animal Medicine (ULAM) and the experimental protocol was approved by the Committee on Use and Care of Animals in accordance with NIH guidelines. An oral diet containing CFZ was fed to mice as previously described (Baik J, et al., Antimicrob. Agents Chemother. 2013; 57:1218-30; Baik J, et al., PLoS One 2012; 7:e47494; Keswani R K, et al., Mol. Pharm. 2015:10). CFZ (C8895; Sigma-Aldrich, St. Louis, Mo., USA) was dissolved in sesame oil (Roland, China, or Shirakiku, Japan) to achieve a concentration of 3 mg/ml, which was mixed with Powdered Lab Diet 5001 (PMI International, Inc., St. Louis, Mo., USA) to produce a 0.03% drug to powdered feed mix. A corresponding amount of sesame oil was mixed with chow for vehicle treatment (control mice). On average, food consumption for a 25 g mouse was 3 g per day, resulting in 10 mg of bioavailable drug/kg per day. For CFZ treatment, the drug diet was administered for 8 weeks followed by regular chow for 8 weeks (washout phase).

Isolation of CLDIs from Mouse Spleen.

At 8 weeks post drug feeding, mice were euthanized by exsanguination while deeply anesthetized by an intraperitoneal injection of ketamine (100 mg/kg)/xylazine (10 mg/kg) and spleens were harvested and cut open to prepare tissue homogenate in phosphate-buffered saline (PBS). The tissue homogenate was sonicated for 30 minutes and centrifuged (100×g for 1 minute) to remove large cell debris. A solution of 10% sucrose in PBS was added to the acquired supernatant and the mixture was centrifuged (100×g). The resulting supernatant was centrifuged (21,000×g for 1 min) to pellet drug inclusions which were then resuspended in 2 ml of 10% sucrose. CLDIs were further purified using a 3-layer discontinuous gradient (50%, 30% and 10% sucrose in PBS) centrifugation method (3200×g for 30 min, no brakes) (Yoon G S, et al., Mol. Pharm. 2015:10). The CFZ content of the isolated CLDIs was determined spectrophotometrically (λ=495 nm) by procuring 100 μl of CLDIs (in triplicate) by centrifugation (21,000×g for 1 min) and dissolution in DMSO followed by comparison with calibrated CFZ standards.

### Fluorimetry.

CFZ was dissolved in DMSO to achieve a concentration of 20 μM. Fluorescence excitation and emission scans were done in increments of 10 nm from 400 nm to 800 nm on a Perkin-Elmer LS-55 fluorescence spectrometer using standard cuvettes. Data were imported into Microsoft® Excel (Redmond, Wash., USA) (MS-Excel) for further analysis. The fluorescence yield was background-subtracted using data obtained from solvent alone (DMSO) and was normalized to the maximum fluorescence yield measured across the spectral wavelength range tested.

### Spectral Confocal Microscopy.

For preparation of slides, CFZ drug crystals were dusted on a glass slide followed by the application of a glass cover slip. For slides of CLDIs, a 20 μl drop of purified CLDIs was placed on a glass slide and allowed to dry overnight in the dark. The following day, a single drop of Prolong® Gold (Life Technologies, Carlsbad, Calif.) was added to the CLDIs and a cover slip was applied prior to imaging. Spectral confocal microscopy was performed on a Leica Inverted SP5X confocal microscope system with 2-photon FLIM (Leica Microsystems Inc., Buffalo Grove, Ill.) using excitation wavelengths (λ=470-670 nm). Image analysis and quantification was performed on Leica LAS AF. Several regions of interest (ROIs) of individual crystals were used to obtain fluorescence data which were imported into MS-Excel for further analysis. All fluorescence yields were normalized to the maximum fluorescence yield measured across the spectral range tested and background subtracted using data obtained from a blank slide.

### Epifluorescence Microscopy.

Visualization of all samples (cells or crystals) was done on a Nikon Eclipse Ti (Nikon Instruments Inc., Melville, N.Y., USA). The fluorescence filters (Excitation/Emission) used were DAPI (350/405 nm, exposure=50 ms), FITC (490/510 nm, exposure=100-500 ms), Texas Red (590/610 nm, exposure<500 ms) and Cy5 (640/670 nm, exposure=15 ms). Brightfield color photographs were acquired using a Nikon DS-Fi2 camera while fluorescence photographs were acquired using a Photometrics® CoolSNAP™ MYO (Photometrics, Tucson, Ariz., USA) camera. CLDIs (seen as intense red pigmentation) were counted and analyzed for physical dimensions using the Nikon Elements software (Nikon Instruments Inc., Melville, N.Y., USA).

### Identification of the CLDI Signal by Flow Cytometry in RAW264.7 Cells.

Macrophages phagocytose CLDIs isolated from mouse spleen following 8 weeks of CFZ treatment (14). RAW 264.7 cells (TIB-71™ ATCC, Manassas, Va.) cells were maintained with DMEM+10% fetal bovine serum (FBS) (10082; Gibco®, Invitrogen, Carlsbad, Calif., USA) with 1% penicillin/streptomycin (15140; Gibco®, Invitrogen, Carlsbad, Calif., USA) at 37° C., 5% CO₂. The cells were seeded at 4×10⁵ cells/well in a 6-well plate 18-20 hours prior to incubation with isolated and purified spleen CLDIs at a solution equivalent concentration of 40 μM CLDIs (14). Following 24 hours post CLDI incubation, cells were gently scraped and suspended in sterile flow cytometry tubes at a density of ˜2×10⁶ cells/ml of phosphate-buffered saline (PBS)+5% FBS. The cells were analyzed on a MoFlo® Astrios™ (Beckman Coulter, Brea, Calif., USA) using various laser combinations. Unless otherwise mentioned, forward and side scatter were measured using the 488 nm laser. Laser settings are referenced in the following format—Excitation Emission/Bandwidth in nm. For example, if the excitation laser used is 488 nm and the emission detector is at 576 nm with a bandwidth of 21 nm, the written format is 488 576/21. To check the activity of viability dyes and signal compensation, just prior to analysis, propidium iodide (PI) (00-6990-50; Affymetrix eBioscience, San Diego, Calif., USA) or 4,6-diamidino-2-phenylindole, dihydrochloride (DAPI) (D1306; Life Technologies, Grand Island, N.Y., USA) was added to the cells (5 μl per 1×10⁶ cells). All gating and analysis was done on at least 10,000 cells (DAPI(−) or PI(−) cell population for viability studies) using FlowJo (FlowJo, LLC, Ashland, Oreg., USA). Statistical analysis of sensitivity and specificity was conducted by acquiring >6 brightfield microscopy images of sorted cell populations on standard microscopy slides and >350 cells were counted for each sorting experiment. Sensitivity and specificity were calculated as follows.

$$Sensitivity = \frac{TP}{TP + FN}$$

$$Specificity = \frac{TN}{TN + FP}$$

Where TP (True Positive)=CLDI(+) in CLDI(+) cell population,
FP (False Positive)=CLDI(−) in CLDI(+) cell population,
TN (True Negative)=CLDI(−) in CLDI(−) cell population,
FN (False Negative)=CLDI(+) in CLDI(−) cell population,

### Peritoneal Lavage.

Peritoneal lavage was done as previously reported before and after the initiation of CFZ or vehicle treatment (1 week, 2 weeks, 4 weeks, 8 weeks and 16 weeks (8 weeks drug feed+8 weeks washout phase)) (16). Mice were euthanized as described above followed by sterilization of the outer skin with 70% ethanol. A small incision was made along the midline of the abdomen followed by abdominal skin retraction up to the thoracic boundary and the animal extremities to expose the intact peritoneal wall. A smaller incision was then made on the peritoneal wall to expose the cavity. The entire peritoneal cavity was washed with ice-cold sterile PBS+5% FBS (5-10 ml) and collected as peritoneal exudate. The exudate was then centrifuged (100×g for 5 min, 4° C.) and resuspended in 1.5 ml of PBS+5% FBS. Cells were counted using a hemocytometer for viable cells using Trypan Blue and for CLDI-containing cells.

### Alveolar Lavage.

Mice were euthanized as described above and the trachea was surgically exposed and cannulated with an 18G luer stub and the lungs were lavaged to obtain alveolar exudate by instilling calcium- and magnesium-free Dulbecco's PBS (DPBS) containing 0.5 mM EDTA in 1-ml aliquots for a total of 6 ml. The alveolar exudate fluid was centrifuged (400×g, 10 min, 4° C.) and resuspended in RPMI 1640 media. Viable (using Trypan Blue staining method) and CLDI containing cells were counted using a hemocytometer.

### Flow Cytometry of Peritoneal and Alveolar Exudate.

Peritoneal and alveolar lavage was performed as stated before to obtain the respective exudates. Purified Fc block CD16/32 (1 μl) was added for every 100,000 cells for analysis. For CLDI fluorescence signal experiments, cells were pelleted by centrifugation (100×g for 5 min) followed by resuspension in PBS+5% FBS (500 μl). For functional assays, antibodies—anti-F4/80-eFluor 450 (48-4801, Affymetrix eBioscience, San Diego, Calif., USA), anti-CD86-FITC (553691, BD Biosciences, San Jose, Calif., USA), anti-CD206-FITC (141704, BioLegend®, San Diego, Calif., USA), anti-CD11c-eFluor450 (48-0114, Affymetrix eBioscience) and anti-Ly6G-eFluor450 (48-5931, Affymetrix eBioscience) were added to the cell suspension at a loading of 1 μg/100,000 cells (1:10 volumetric ratio) and incubated in the dark (30 min, 4° C.). Following incubation, the samples were diluted with PBS and pelleted by centrifugation (100×g, 5 min). The supernatant was discarded and the pellet was re-suspended in 300 μl of PBS+2% FBS. Just prior to analysis, PI (5 μl per 1×10⁶ cells) was added to the cells to assess viability. Sample measurements were done on a MoFlo® Astrios™ EQ. All gating and analysis was done on at least 10,000 live cells (PI(−) cell population) using FlowJo. CLDI(+) cells were gated either using 640 671/30 or 640 795/70. A small sub-population of CD206(+) cells, which was present in both CLDI(+) F4/80(−) and CLDI(−) F4/80(+) populations, exhibited high CD206 expression based on the intensity of the fluorescence signal. These cells also had extremely high side-scatter that were measured as saturated signals. This small sub-population of cells were not considered for evaluation of change of CD206 expression in the peritoneal macrophages.

### Laser-Scanning Confocal Microscopy.

CLDI treated RAW264.7 cells were generated as mentioned above but on cover-slips in a 6-well plate. The cells were washed with PBS buffer following which Hoechst 33342 (Invitrogen, Carlsbad, Calif.) and FM® 1-43 (Molecular Probes T35356, Invitrogen) were used to stain cell nuclei and the plasma membrane, respectively. Cells were incubated with 1:1 (v/v) dye mixtures of 5 μg/ml Hoechst 33342 and 7 μM FM® 1-43 in HBSS (300 μl) for 15 min at room temperature. The confocal imaging of the live cells was performed on an Olympus Fluoview 500 (Olympus America Inc., Center Valley, Pa., USA) using lasers for DAPI (405 450/50), FITC (488 525/50) and Cy5 (640 671/30) channels. Z-stack images of the cells were captured along the Z-axis (interval=0.25 μm) and analyzed using the Nikon NIS-Elements 3.2 confocal software (Nikon Instruments Inc., Melville, N.Y.). For peritoneal macrophages stained with F4/80, a drop of the stained cell sample was mounted on a blank microscopy slide; Prolong Gold and a cover-slip were applied followed by immediate scanning.

### Data Plotting and Statistical Analysis.

Plots were constructed using Origin 9.0 (OriginLab Corporation, Northampton, Mass., USA) and laid out in figure format using scalable vector graphics format (svg) in either Inkscape or GIMP. Flow cytometry plots were obtained in svg format directly from FlowJo and assembled using Inkscape. All statistical analysis was performed using Student's t-test in MS-Excel. Correlation statistics were done using a Pearson's test in Origin 9.0. Results were considered significant if p≦0.05. MIFlowCyt compatible information was prepared using established standards Cytom. Part A 2008; 73:926-930).

### Results

### Fluorescence Spectral Analysis of CFZ and CLDIs

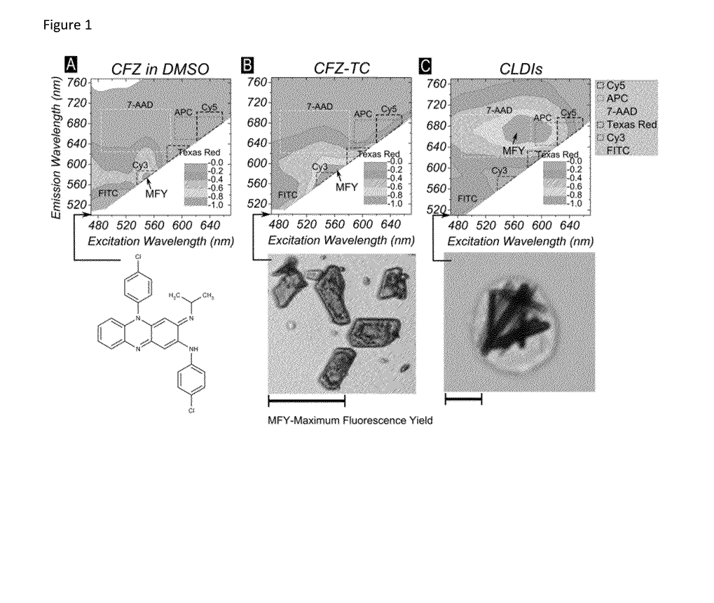

The fluorescence excitation and emission scan of CFZ dissolved in DMSO indicates that CFZ in solution is fluorescent in the range—Excitation: 540-560 nm, Emission: 560-600 nm (FIG. 1A). To characterize the fluorescence of CFZ crystallized as free-base triclinic crystal (closest triclinic polymorph of neutral CFZ crystal, CCDC refcode: DAKXUI01), spectral confocal microscopy on individual CFZ-TC crystals dispersed on a microscopy slide was used. CFZ-TC has a similar fluorescence profile as soluble CFZ (FIG. 1B). However, residual fluorescence of CFZ-TC is also detected at lower (Ex: 480-500 nm, Em: 510-540 nm) and higher (Ex: 580-600 nm, Em: 600-640 nm) spectral ranges. To characterize and identify a specific spectral signature associated with the intracellular accumulated and crystallized CFZ-CLDIs, the spleens of 8-week drug-fed mice were harvested and CLDIs were isolated and purified. By visual inspection, CLDIs were dark red, rod shaped particles that were distinct from the orange-red CFZ-TC crystals (FIG. 1B-C, inset photographs). When subjected to fluorescence spectral analysis using the spectral confocal microscope, CLDIs exhibited a red-shift in their emission fluorescent spectra with peak fluorescence activity at Ex: 560-600 nm, Em: 650-690 nm (FIG. 1C). To place this specific red-shift in the context of other commonly used dyes, CFZ-TC exhibited a fluorescence profile similar to Fluorescein Isothiocyanate (FITC), Cyanine 3 (Cy3), Rhodamine and Texas Red (TR) whereas CLDIs had a red-shifted fluorescence profile similar to TR, Cyanine 5 (Cy5), 7-Aminoactinomycin D, (7-AAD), Allophycocyanin (APC) and other commercial Cy5 derivatives.

Identification of CLDI-Containing Cell Subpopulations with Standard Flow Cytometer Configurations.

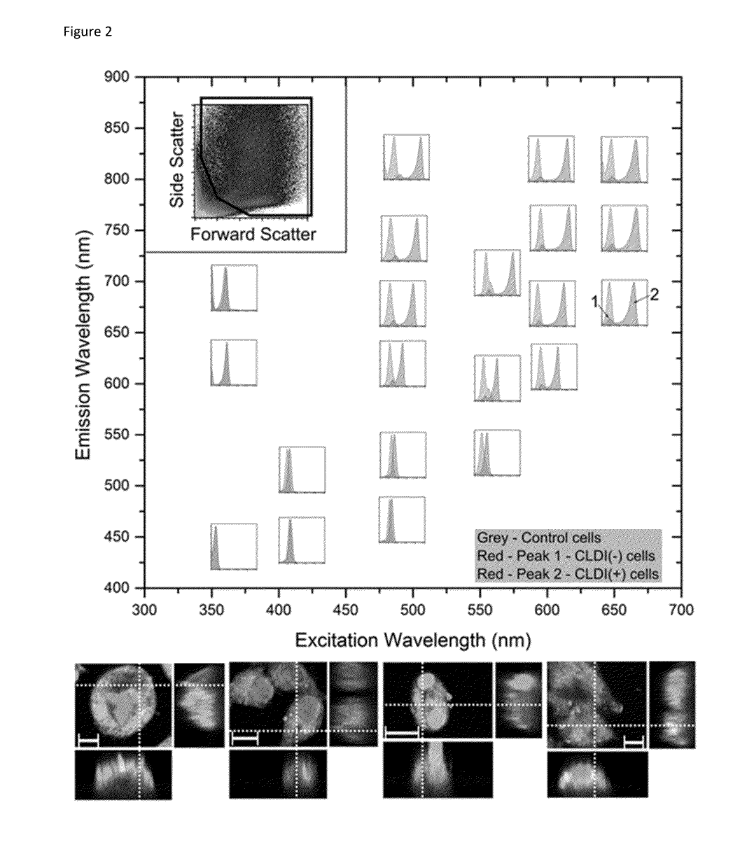

To design a single-cell analysis technique to study the pharmacology of xenobiotic-sequestering macrophages and the impact of CLDIs on cellular functions, it was determined whether flow cytometery could be used to distinguish between cells that contain and do not contain CLDIs. Samples were analyzed using standard excitation lasers and detector configurations, to determine the specific fluorescence cytometry settings that can be used for analyzing CLDI containing cell subpopulations and to establish any spectral overlap detected in the other channels (FIG. 2). The use of ultraviolet (355 nm) and violet (405 nm) excitation lasers resulted in the detection of single populations at all emission detector settings, corresponding to the background autofluorescence signal of unlabeled cells. Similarly, a single cell population was detected when samples were excited with the blue (488 nm) or green (561 nm) excitation lasers at emission detection <600 nm. However, at emission detection >600 nm, two distinct cell populations were observed, one which corresponded to the background fluorescence signal of CLDI-free cells (labeled 1), and the other which corresponded to the positive fluorescence signal of the CLDI containing cells (labeled 2). Furthermore, the use of the red (592 nm) and far-red (640 nm) excitation laser resulted in the detection of two cell sub-populations corresponding to the CLDI-free and CLDI-containing cells, at all tested emission detector settings. The laser and detector settings that detected the subpopulation of CLDI-containing cells corresponded to the CLDI fluorescence spectra, which were independently measured using the spectral confocal microscope (FIG. 1C).

To confirm these observations, a fluorescence activated cells sorting experiment was performed using 488 664/22, 561 692/75 and 640 671/30 along with laser-scanning confocal microscopy of incubated cells. When RAW264.7 cells incubated with CLDIs and labeled with fluorescent cellular staining dyes—Hoechst 33342 (nucleus) and FM 1-43 (plasma membrane) were imaged, the intracellular accumulation of CLDIs was confirmed via their far-red fluorescence (FIG. 2, bottom right inset) distinct from the green fluorescence of FM 1-43 and blue fluorescence of Hoechst 33342. Additionally, as per brightfield microscopy, the sorted cell population with the lower fluorescence signal corresponded to CLDI-free cells whereas the sorted cell population within the high fluorescence signal corresponded to CLDI-containing cells (FIG. 2A). The sorting sensitivity was 98.34%, 98.82% and 99.01% at 488 664/22, 561 692/75 and 640 671/30, respectively, whereas sorting specificity was 86.30%, 90.80% and 93.98%, respectively (See Methods for formula). The detection of CLDIs in subsequent flow cytometry experiments was done using either 640 671/30 or 640 795/70 whereas for microscopy, CLDIs were detected using the red laser (Excitation: 640 nm, Emission: 670 nm).

### Functional Flow Cytometric Analysis of Live Vs Dead, CLDI-Containing Cells

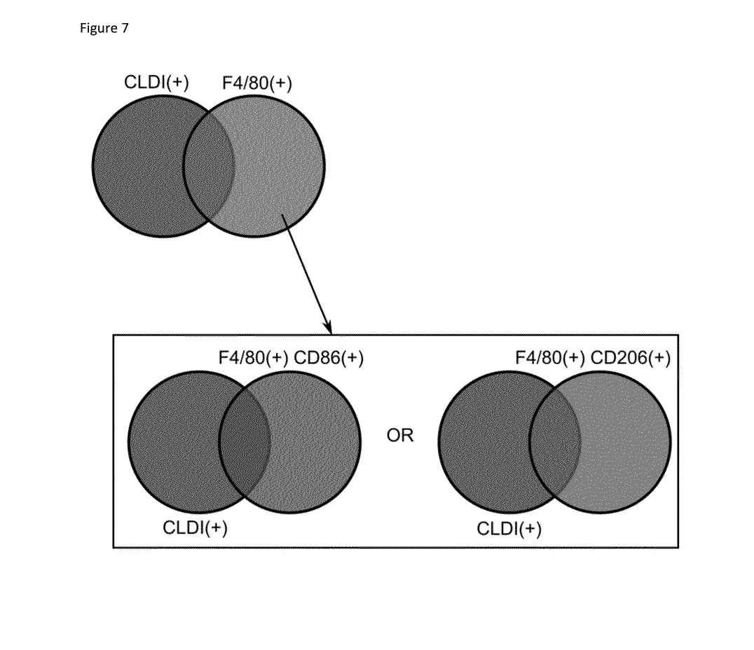

In flow cytometry experiments, a fundamental functional assay involves discriminating live and dead cells using standard fluorescent labels. Accordingly, to determine whether CLDI-containing cells were viable, RAW264.7 cells loaded with CLDIs were further incubated with membrane impermeant cell viability dyes—PI or DAPI prior to flow cytometric analysis. Using the laser-detector settings for PI—561 614/20, two populations were detected using CLDI-incubated RAW264.7 cells. A compensation of the spectral overlap that was observed at 561 614/20 was conducted to verify that PI can be used in the presence of CLDIs (at 640 671/30) without signal overlap (Figure B). Incubating control (CLDI-free) cells with PI yielded two distinct cell populations, corresponding to live (low fluorescence signal) and dead (high fluorescence signal) cells (FIG. 2B). In contrast, after CLDI-containing cell samples were incubated with PI, four populations were observed, corresponding to viable CLDI-free (Live CLDI(−)) and viable CLDI-containing cells (Live CLDI(+)), and dead CLDI-free (Dead CLDI(−)) and dead CLDI-containing cells ((Dead CLDI(+)) (Figure B, FIG. 7). In the same manner, flow cytometric analysis revealed that DAPI could be used to determine the viability of cells containing CLDIs (Figure C). With DAPI, four distinct populations were observed (without compensation), corresponding to live (weak UV signal) and dead (strong UV signal), CLDI containing (marked as CLDI(+)) and CLDI free (marked as CLDI(−)) cells. Thus, both PI and DAPI were suitable as fluorescent markers for discriminating between live and dead cell subpopulations of CLDI-containing cells.

In Vivo Flow Cytometry Detection of CLDI-Containing Cells in Peritoneal and Alveolar Exudates from CFZ-Treated Mice

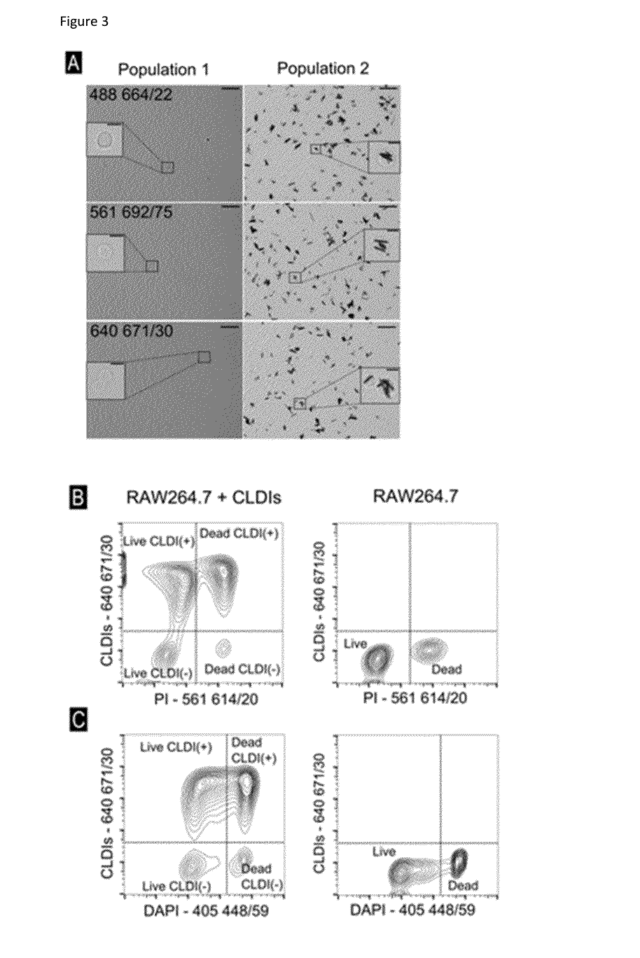

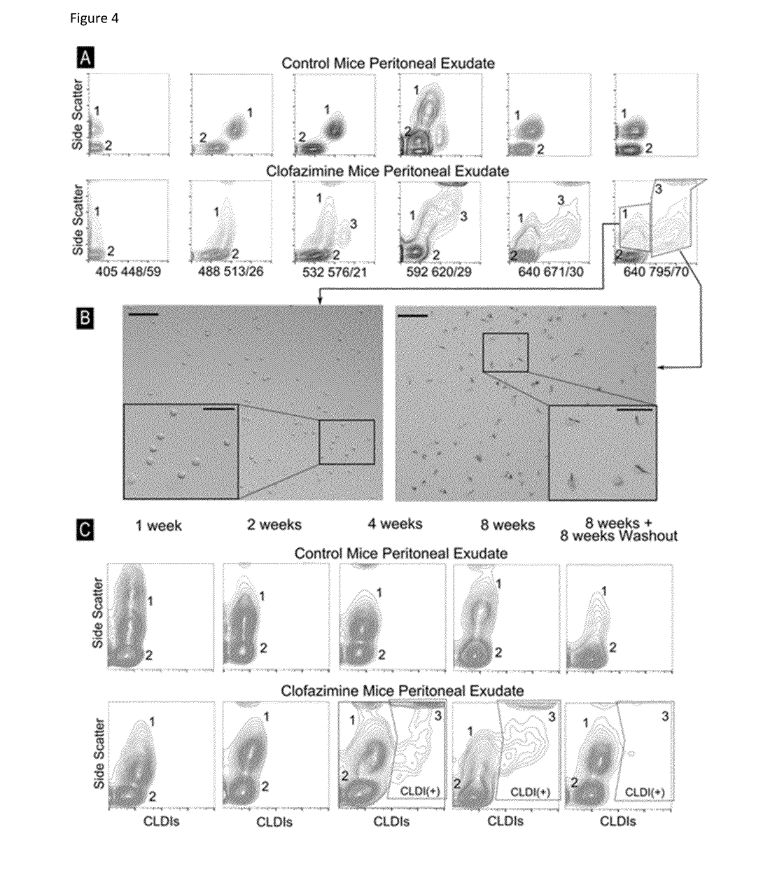

To measure the presence of CLDIs in peritoneal exudates obtained from CFZ-treated mice, a quantitative, flow cytometric analysis was performed on peritoneal exudate of mice following 8-week drug administration. While the control mouse peritoneal exudate showed two distinct populations (labeled 1 and 2—FIG. 3A) at all tested laser settings, the CFZ mouse peritoneal exudate showed two populations when assayed with the 405 448/59 or 488 513/26 laser-detector combination. With the use of 532 576/21, 592 620/29, 640 671/30 and 640 795/70, however, three distinct populations were observed (labeled 1, 2 and 3—FIG. 4A). The mean fluorescence intensity of population 3 was increased >100-fold relative to population 1 at 640 671/30 and 640 795/70. To confirm that the third population was CLDI(+) cells, the cell populations labeled 1 and 3 was sorted using the 640 795/70 laser-detector combination and plated them in cell culture media overnight. Population 1 was confirmed to be CLDI(−) cells whereas population 3 was indeed CLDI(+) cells (FIG. 4B).

Next, it was determined whether the time course of CLDI accumulation in macrophages as determined by flow cytometry corresponded to the expected time course of CLDI accumulation following drug treatment (Baik, 2013, supra; Baik 2012, supra). Following drug treatment of mice for varying time periods, peritoneal exudates were obtained and analyzed by flow cytometry as well as light microscopy, revealing that CLDIs were not present at 1 week and 2 weeks, but could be detected starting at 4 weeks (FIG. 4C).

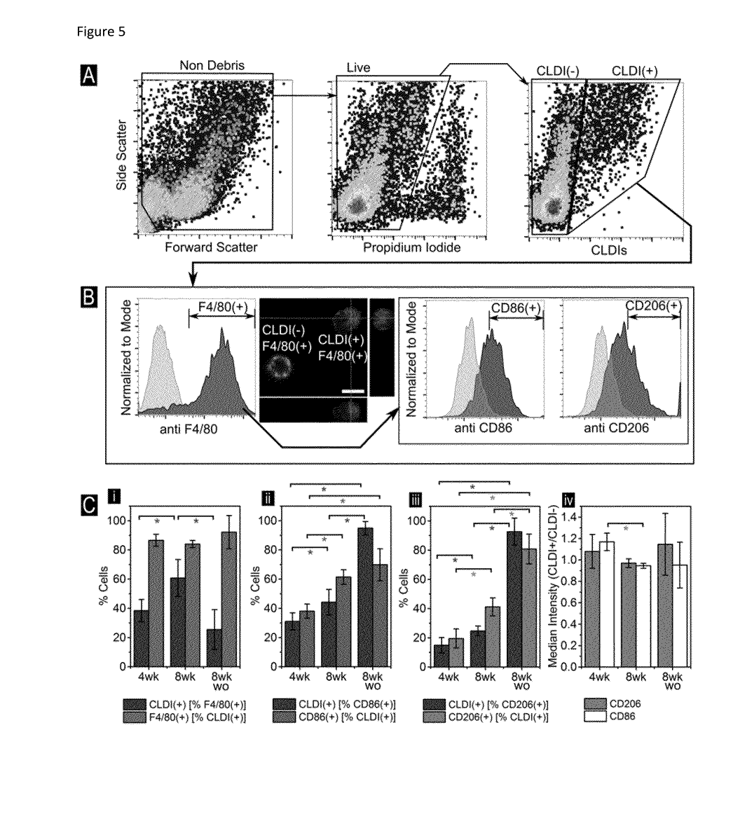

To determine whether flow cytometry could be used to characterize the molecular phenotype of CLDI-containing macrophage sub-populations, multi-stain flow cytometry was conducted on peritoneal exudates of CFZ-treated animals. For this purpose, cells obtained from peritoneal exudates were incubated with other commonly employed macrophage-targeted antibodies possessing fluorescent signals distinct from that of CLDIs (405 448/59 and 488 513/26; FIG. 1). Since CLDI accumulation has been reported to be macrophage targeted (3,18-20), the murine pan-macrophage marker F4/80 as well as other macrophage phenotype surface markers—CD86 and CD206 were chosen (Figures). The schematic for quantification of the gating strategy is shown in FIG. 5A, B and FIG. 7. The analysis revealed that in peritoneal exudates, CLDIs were primarily localized to F4/80(+) peritoneal macrophages (PMΦ) (>85% of total CLDI(+) cells at all measured time points (FIG. 4B, no statistically significant changes across the times measured). This observation was also confirmed via laser-scanning confocal microscopy using the far-red fluorescence region for CLDIs and the violet laser to visualize F4/80 extracellular membrane staining (FIG. 4B). The accumulation, however, was both treatment and time dependent with respect to F4/80(+) PMΦ as ˜38% of F4/80(+) PMΦ were CLDI(+) after 4 weeks of CFZ treatment which increased to ˜61% after 8 weeks (p<0.05) of treatment and declined to ˜25% at 8 weeks of washout (p<0.05; FIG. 4C(i)). Within F4/80(+) CD86(+) cells, CLDI accumulation increased in a time-dependent fashion, from ˜31% after 4 weeks of CFZ treatment to ˜95% at 8 weeks+8 weeks washout (p<0.05, FIG. 4C(ii)). As a proportion of F4/80(+) CD206(+) cells, CLDI(+)% increased from ˜15% after 4 weeks of CFZ treatment to ˜93% after 8 weeks+8 weeks washout (p<0.05, FIG. 4C(iii)). Collectively, these findings indicate that CLDI accumulation was not restricted to the CD86(+) or CD206(+) macrophage phenotype (FIG. 4B). In a related trend, % CLDI(+) cells that could be identified as F4/80(+) CD86(+) increased from ˜38% at 4 weeks post-drug feeding to ˜70% at 8 weeks+8 weeks washout (p<0.05) whereas % CLDI(+) cells that could be identified as F4/80(+) CD206(+) increased from ˜20% at 4 weeks post-drug feeding to ˜81% at 8 weeks+8 weeks washout (p<0.05). While the accumulation of CLDIs in CD86(+) cells was higher relative to CD206(+) cells at 4 weeks and 8 weeks post-drug feeding (p<0.05), the accumulation was not statistically deviated towards any phenotype at 8 weeks+8 weeks washout (p>0.05). In contrast, the ratiometric CD86 median surface expression in CLDI(+) relative to CLDI(−) decreased from ˜1.17 at 4 weeks post-drug feeding to 0.95 (p<0.05, FIG. C(iv)) at 8 weeks post-drug feeding. The ratiometric CD206 median surface expression in CLDI(+) relative to CLDI(−) CD206 remained between ˜0.97 and ˜1.15 for CD206 with no statistically significant difference at the three measured time points (FIG. 5C(iv)). Since the fold-change in the median surface expression is much less than the benchmark of two-fold change in expression to ascertain significant change in expression of the two surface markers—the changes, as a result of being CLDI(+) reflect relatively minor expressional differences between the two populations. Finally, the viability of F4/80(+) cells, as measured via PI(−) cells, was unaffected by the presence of CLDIs (viability of CLDI(+) F4/80(+)=87±6%, CLDI(−) F4/80(+)=88±12%, p=0.84 between the two groups, n=4). The viability measured via flow cytometry also agreed with the sample viability of CLDI+ cells in the peritoneal exudate measured by Trypan blue staining (87±9%, n=4).

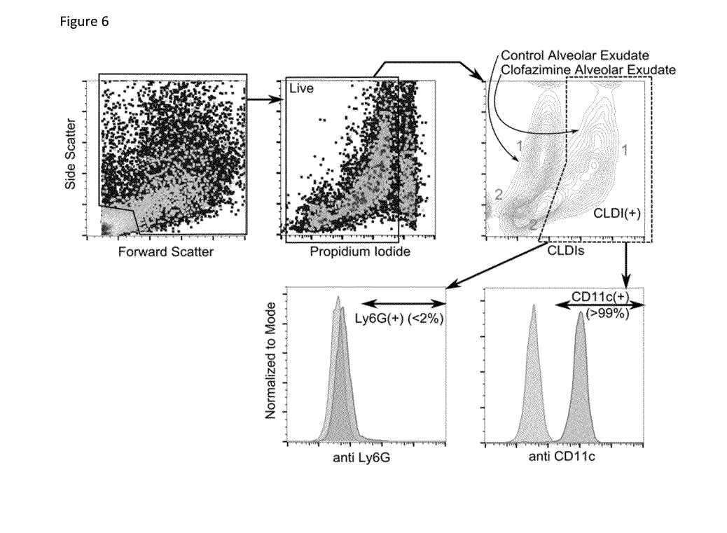

To follow up on the analysis on peritoneal exudate, a flow cytometric analysis was performed on alveolar exudate of drug fed mice at 8 weeks to confirm the macrophage-specific accumulation of CLDIs in the lung (Baik et al., 2012, supra; Conalty M L, Jina A G. The Antileprosy Agent Clofazimine (B.663) in Macrophages: Light, Electron Microscopy and Function Studies. In: Reticuloendothel. Syst. Immune Phenom. Springer; 1971. p 323-331; Conalty M L, et al., Br. J. Exp. Pathol. 1962; 43:650-654) (FIG. 6 shows 6). Both the control and CFZ mice alveolar exudates had two distinct populations. However, population 2, that had high side-scatter, had a distinctly higher fluorescence at the settings used to detect CLDIs (640 671/30), marking them out as CLDI(+) cells. Upon further testing with fluorescent antibodies (at 405 448/59), specific for alveolar macrophages (CD11c) and neutrophils (Ly6G) (21), CLDI(+) cells were determined to be overwhelmingly CD11c(+) (>99%) whereas <2% of Ly6G(+) cells were detected as CLDI+.

Correlation of CLDI Amount with Mean Cellular Fluorescence Intensity (MFI)

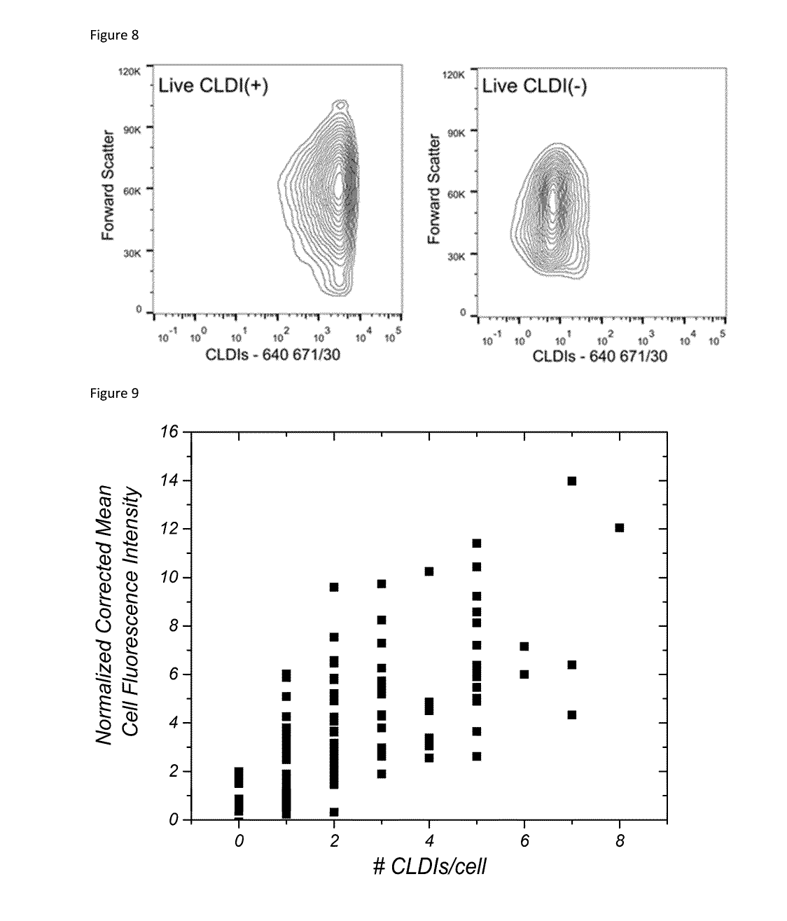

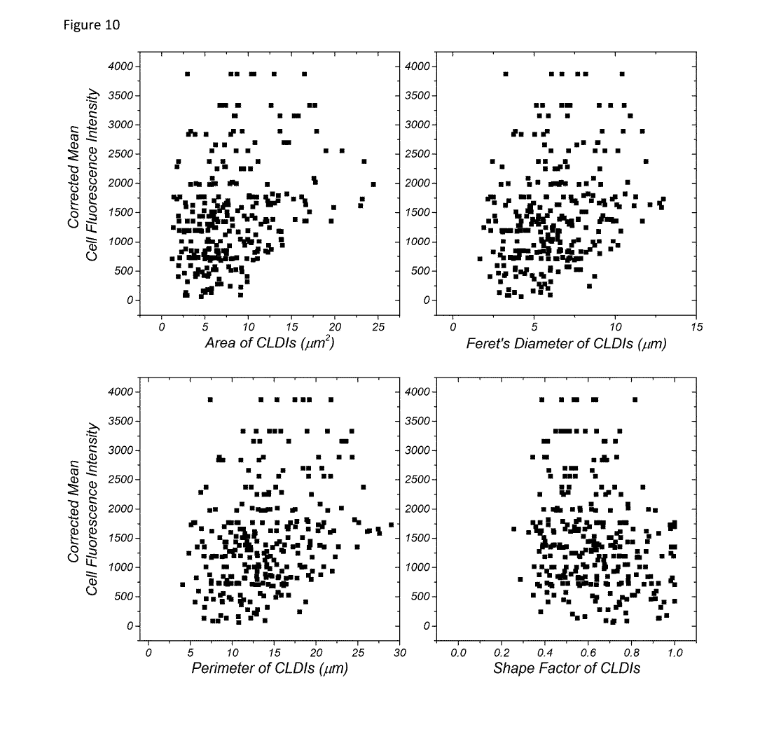

In fluorescence activated cell sorting experiments, attempts to relate the MFI of sorted cell populations with the total number of CLDIs per cell did not yield interpretable data. Therefore, to determine whether the fluorescence signal per cell was related to the number of CLDIs in each cell, RAW264.7 macrophage cells were incubated with CLDIs, and fluorescence images were acquired with conventional epifluorescence microscopy using the Cy5 filter for CLDIs (FIG. 9). The normalized background corrected MFI of cells was strongly correlated (Pearson's r=0.70, p<0.0001) with the number of CLDIs/cell. As expected, cell MFI was uncorrelated to intracellular CLDI geometry and morphology (FIG. 10, Pearson's r=−0.2 to 0.3, p-value <0.005). The resulting fluorescence signal readily distinguished CLDI(+) cell relative to a CLDI(−) cells (p-values <0.005). Cells containing 1 vs 2 CLDIs were also distinguishable (p<0.05). However, cells containing >2 CLDIs could not be distinguished from cells containing fewer CLDIs, in a statistically relevant manner. Given that each CLDI contains ˜25 femtomoles CFZ (24.3±13.5, n=8 mice), it was determined that this method could be used to quantify CFZ sequestration in CLDIs, up to 50 femtomoles of CFZ (corresponding to 2 CLDIs per cell).

In summary, the analysis of macrophage populations in vitro and ex vivo was made possible due to the unique fluorescence spectral characteristics of CLDIs relative to neutral free base CFZ precipitates as well as the CFZ molecule in soluble form. The specific fluorescence of CLDIs was utilized in single-cell flow cytometry to study macrophage-related intracellular drug accumulation wherein it was possible to measure macrophage functions including viability and expression of different cell surface markers, by exploiting the unique fluorescence spectrum of CLDIs and the availability of many other orthogonal fluorescent probes that can be detected with standard flow cytometry parameters, with minimal spectral overlap from the CLDI signal. It was also confirmed that the presence of CLDIs has a minor polarizing effect towards the M2 macrophage phenotype. More importantly, the unique fluorescence shift of CFZ molecules from the freely soluble green-blue fluorescent state to the solid far-red CLDI fluorescent state facilitates analysis of the molecular mechanisms driving drug accumulation and CLDI formation, using a variety of molecular, pharmacological, genetic, or systems biology approaches. These studies are useful for furthering understanding of the role of xenobiotic sequestering macrophages in the response of the organism to bio-accumulating drugs or environmental toxicants.

### Example 2

### Clofazimine as a Macrophage-Targeting Photoacoustic Contrast Agent

### Methods

### Screening of FDA-Approved Drugs.

The initial screening to obtain a short-list of drugs from Drugbank was done via Boolean search to extract all small molecule drugs that were FDA-approved, not withdrawn nor illicit and containing the descriptive term “anti-inflammatory” resulting in a short list of 126 molecules. Two 1536-well plates containing a common subset of the NCGC Pharmaceutical Collection (NPC) (Huang, R. et al. Sci. Transl. Med. 3, 80ps16 (2011)) were selected to be analyzed for visible color. The plates included 87 short-listed drugs solubilized in dimethyl sulfoxide (DMSO) up to a concentration of 20 mM that were then photographed using a flatbed scanner and measured for their UV/Vis absorbance profiles. The raw PNG formatted image was manually cropped and stored as 2 separate PNG images. A simple custom Java program was then used to select the centremost pixel from each well and report the colour information in both RGB and HSV format. Molecules not yet available in NPC format (39 in number) were searched in literature, USP Pharmacopeia and other databases for their colour and absorption spectra. To re-evaluate the UV/Vis profile of the 10 pigmented compounds, samples were prepared at a concentration of 0.2-1 mM in DMSO and spectra was recorded from 300-1000 nm on a Biotek II multi-well plate reader. The solution absorbance was normalised with the corresponding value using a control sample containing DMSO alone and plotted from 300 to 600 nm as no discernible absorbance was measured for any of these compounds at >600 nm.

### PA Spectroscopy and Sample Preparation.

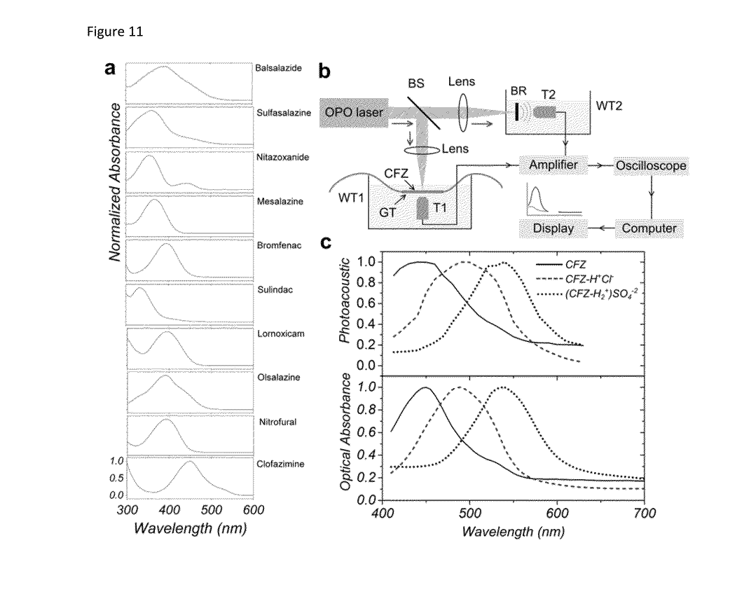

The setup for PA spectroscopy measurement is shown in FIG. 11b. The laser source is a tunable OPO (Surelite OPO Plus, Continuum) pumped by the third harmonic of an Nd:YAG laser (Surelite, Continuum). The OPO laser, working at a repetition rate of 10 Hz, can provide a super broad tuning range of 410-650 nm, 710-2500 nm. The emitted laser was separated into two parts by a beam splitter (BS) with a splitting ratio of 90:10. The reflected beam was focused on the sample by a lens. The sample was placed in a transparent glass tube (Kimble Chase North America) with an inside diameter 1.1 mm and a wall thickness 0.2 mm, which was submerged in a water tank (WT). The transmitted beam was focused on a black rubber, which could absorb all incident laser energy and was used as a reference for laser pulse energy calibration. The generated PA signals from the sample and the black rubber were received by two transducers T1 and T2 (C323, Olympus NDT), amplified by a pulser and receiver (5072R, Olympus NDT), digitized by an oscilloscope (TDS 540A, Tektronix) and finally collected by a computer. PA signal intensity (p) at a specific wavelength λ is linearly proportional to the corresponding absorption coefficient μₐ(λ) of the sample. Mathematically, this can be formulated as

p(λ)=ΓΦμₐ(λ)

where Γ is the Gruneisen parameter, which is a constant and Φ is the laser fluence, which was calibrated using the reference black body (Feng, T. et al. Ting. Opt. Lett. 40, 1721-1724 (2015)). Clofazimine (CFZ) as a free base was obtained directly from Sigma-Aldrich (St. Louis, Mo., Catalogue No. C8895) and solubilised in DMSO and confirmed via ¹H-NMR to be unprotonated in solution (Keswani, R. K. et al. Mol. Pharm. 12, 2528-2536 (2015)). To obtain CFZ-H⁺Cl⁻ in solution, CFZ-HCl crystals, obtained via crystallisation at pH 5 as published before (Keswani, R. K. et al. Mol. Pharm. 12, 2528-2536 (2015), were resolubilized in DMSO. Briefly, equal volumes of 1 M NH₄Cl in H₂O and 2 mM CFZ in methanol were mixed with the addition of a surfactant (up to 2% (v/v) Triton™ X-100, Sigma-Aldrich, Catalogue No. X100) and incubated at room temperature for 48 hours. The precipitates obtained were washed twice with H₂O and lyophilized followed by storage at −20° C. until used. Just prior to analysis, CFZ-H⁺Cl⁻ crystals were solubilized in DMSO. These solubilized samples were also confirmed to be monoprotonated via ¹H-NMR. For diprotonated CFZ, free-based CFZ crystals were dissolved in 9 M H₂SO₄ resulting in the formation of a deep purple coloured solution of diprotonated CFZ.

### In Vivo CFZ Model for Oral Feeding.

Experiments using the CFZ mice model were performed as published before (Baik et al., 2013, supra; Baik et al., 2012, supra; Keswani, R. K. et al. Mol. Pharm. 12, 2528-2536 (2015)). Mice (4 week old, male C57Bl/6) were purchased from the Jackson Laboratory (Bar Harbor, Me.) and acclimatised for 2 weeks in a specific-pathogen-free animal facility. All animal care was provided by the University of Michigan's Unit for Laboratory Animal Medicine (ULAM), and the experimental protocol was approved by the Committee on Use and Care of Animals. CFZ was dissolved in sesame oil (Roland, China, or Shirakiku, Japan) to achieve a concentration of 3 mg/ml, which was mixed with Powdered Lab Diet 5001 (PMI International, Inc., St. Louis, Mo.) to produce a 0.03% drug to powdered feed mix. A corresponding amount of sesame oil was mixed with chow for vehicle treatment (control).

### In Vitro Cell-Line Experiments.

The murine MΦ cell line RAW264.7 was purchased from ATCC (Manassas, Va.) and maintained in growth media (DMEM (Life Technologies, Carlsbad, Calif.) supplemented with 10% FBS and 1% penicillin/streptomycin). Isolated CLDIs were added at 20 μM solution equivalent concentration in growth media to 8-well chamber-slides (Nunc™ Labtek™ II, Thermo-Scientific Catalogue No. 154534) containing 5×10⁴ RAW264.7 cells/well and incubated (24 h at 37° C. and 5% CO₂). Following incubation, cells were fixed using 4% paraformaldehyde in PBS for 15 mins at room temperature followed by rinsing and washing with ice-cold PBS twice. The samples were counterstained with Hoechst33342 for nuclear labelling followed by removal of chambers. A drop of Prolong Gold® (Life Technologies) was placed on the sample and covered with a glass coverslip for imaging.

### PA Microscopy Setup.

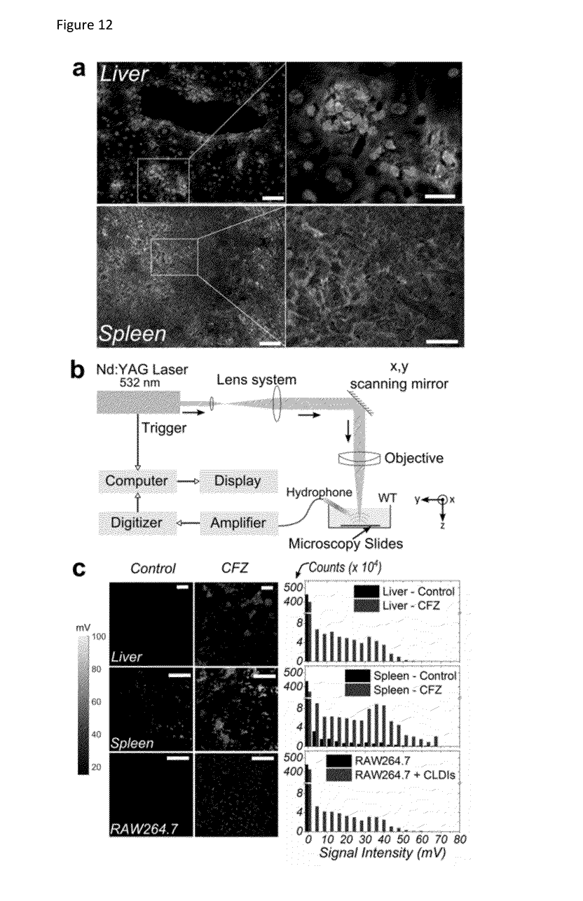

The setup for PA microscopy measurement as described previously is shown in FIG. 12b. The laser source is a diode-pumped solid-state Nd:YAG laser (Spot-10-200-532, Elforlight Ltd, UK), with a wavelength of 532 nm and a pulse duration of 2 ns. The emitted laser was first collimated by a lens system, then reflected by a two-dimensional (x, y) scanning mirror (6230H, Cambridge Technology), and finally, focused on the sample by an achromatic objective (AC254-040-A, ThorLabs) with a focal length of 40 mm. The microscopy slides or chambers submerged in phosphate buffered saline (PBS) buffer, absorbed the green laser and produced acoustic signals. The generated signals were captured by a hydrophone (centre frequency-35 MHz, 6 dB bandwidth 100%), amplified by a low-noise amplifier (ZFL-500LN, Mini-Circuits), digitized by an A/D card (Cobra CompuScope CS22G8, GaGe), transferred to the computer, and finally reconstructed (as signals in mV) using the maximum amplitude projection (MAP) algorithm for visualization (Xie, Z. et al. Opt. Express 19, 9027-9034 (2011)). The calibrated lateral resolution of the system was 2 μm. The contrast-to-noise ratio (CNR) of the images was calculated by the expression

$$CNR = 20 {log}_{10} ( \frac{\vert {μ}_{s} - {μ}_{b} \vert}{\sqrt{{σ}_{s}^{2} + {σ}_{b}^{2}}} )$$

where μₛ and μ_{b} are the means of the signals in the region of interest (ROI) and the background respectively and σₛ and σ_{b} are the corresponding standard deviations. Signal intensity histograms of the acquired images were constructed via measurements in ImageJ (National Institutes of Health, Bethesda, Md.)

Mice Carrageenan Footpad Edema Model. Carrageenan (C1867, Sigma-Aldrich®, 2% in PBS, 30 μl) was injected into the right footpad while PBS (30 μl) was injected in the left footpad as a negative control. For preliminary testing of CFZ as an anti-inflammatory agent, 8-week orally fed mice (CFZ and control) were used. For testing of CFZ in soluble and microcrystal form, naïve 4-5 week old mice were injected with carrageenan first followed by injection of CFZ-A (2 mM in DMSO, 30 μl), CLDIs (2 mM equivalent in PBS, 30 μl) or CFZ-HCl (2 mM equivalent in PBS, 30 μl) at 48 h post carrageenan injection. For control experiments, CFZ-A, CLDIs and CFZ-HCl were injected into the right footpads of naïve 4-5 week old mice. DMSO was used as a control for CFZ-A in the left footpad whereas PBS was used as a control for CLDIs and CFZ-HCl. Footpads were measured (length—l, width—w, three thicknesses—t₁, t₂, t₃) at various time points using a digital vernier calliper and changes in the swelling based on volume of the foot was calculated as per above.

### Synthesis of Gelatin Phantoms.

8% gelatin from porcine skin (G2500, Sigma) was dissolved in water, and then heated and stirred on a hotplate for about one hour. After cooling down, several wells were dug out of the surface and filled with CFZ samples. After that, smaller 8% gelatin phantoms was used to cover the surface of the previous one to seal the embedded samples.

### Clinical Human Model Experiments.

A cadaver hand was requested from Anatomical Donation Program at University of Michigan through an organ donation program. Once received, the cadaver tissue was frozen in a lab specimen freezer under −20° C. Before the imaging experiment, it was submerged in flowing cold water and thawed for about one hour. The request, transportation, storage, and handling of the cadaver tissue followed the policies and guidelines of Michigan Anatomical Gift Law Public Act 368 of 1978, amended as Public Act 39 of 2008. After the experiment, the cadaver tissue was returned to the Anatomical Donation Program.

### PA Tomography of Synthetic Phantoms and Cadaver Hand.

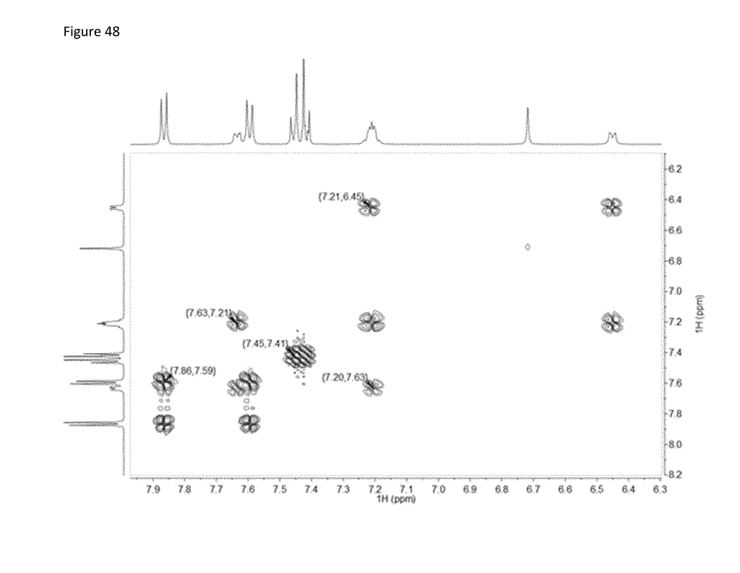

The details of the hardware used have been reported in previous publications (Xu, G. et al. J. Biomed. Opt. 18, 010502 (2013); Yuan, J. et al. J. Biomed. Opt. 18, 86001 (2013)) and a schematic and setup is shown in FIG. 48a,b. The dual-modality platform was built based on the commercial Verasonics® (VSX) system (Vantage 256, Verasonics). In the PA imaging configuration, the second harmonic output (i.e., 532 nm) of an Nd:YAG laser (Powerlite DLS 8000, Continuum) with a pulse duration of 6 ns and a repetition rate of 10 Hz was used as the laser source. In both the phantom and cadaver tissue experiments, ultrasound gel (MediChoice, The Medical Supply Group, Atlanta, Ga.) was topically applied for better acoustic matching between the transducer and the samples. The emitted laser was delivered to the sample through an optical fiber bundle. The excited PA waves from the sample, which propagated through ultrasound coupling gel, was reflected by a transparent glass slide (acoustic reflector, AR) and then received by a transducer array (L7-4 for phantom and CL15-7 for cadaver tissue, Philips Healthcare). The captured radio-frequency (RF) signals were amplified and digitized by the Verasonics system, then transferred to the computer for reconstruction and visualization. Since a graphics processing unit (GPU) was employed to facilitate parallel computation, the dual-modality system can acquire, reconstruct and display two-dimensional PA images with a frame rate up to 10 Hz, which is only limited by the pulse repetition rate of the laser. In US imaging configuration, the Verasonics system drives the transducer array to emit ultrasound waveforms and receive echoes to form ultrasound images. The acquired RF signals were transferred to the computer, reconstructed using MATLAB® using previously published algorithms (Xu, M. & Wang, L. V. Phys. Rev. E 71, 016706 (2005)) and displayed on the monitor. A function generator (FG, 3314A, Hewlett-Packard) was used to generate separate trigger signals for PA imaging and US imaging to ensure the correct timing.

For the clinical human model imaging, a 23 G syringe needle with a nominal outer diameter of 0.64 mm was inserted into the target metacarpophalangeal (MCP) joint of the cadaver hand transcutaneously, guided by the US-PA dual-modality imaging system with its tip stopped next to the joint about 5.6 mm beneath the skin surface. A commercial ultrasound machine (z.one ZONARE) equipped with a linear probe (L10-5, ZONARE) was used to further confirm the positioning of the needle in the joint. CFZ-A at 5 mM in DMSO solution was injected slowly into the joint via the inserted syringe needle. The ROI and background in the reconstructed PA images were segmented and colored based on different color schemes for the purpose of illustration. The laser fluence on the skin surface was estimated to be 4 mJ/cm², which is well below the American National Standards Institute (ANSI) safety limit at 532 nm. The intensities of both the phantom images and the human model images were normalized.

### Results

Photoacoustic (PA) detection relies on intrinsic absorption at specific excitation laser-generated wavelengths resulting in ultrasonic waves detected via conventional acoustic transducers (Wang, X., et al., Biomed. Opt. Express 1, 1117-1126 (2010)). For imaging applications, longer wavelengths are advantageous because they afford greater imaging depth, with reduced potential for phototoxicity (Ntziachristos, V. Nat. Methods 7, 603-14 (2010)). Therefore, to search for candidate small molecule drugs with potential applications as PA contrast agents, the optical properties of FDA-approved anti-inflammatory drugs were assayed. Every organic molecule has a characteristic optical absorption spectrum with colored or pigmented compounds absorbing more light at higher wavelengths relative to colorless compounds (Lewis, G. N. & Calvin, M. Chem. Rev. 25, 273-328 (1939); Sklar, A. L. J. Chem. Phys. 5, 669-681 (1937)). After identifying drugs with the strongest optical absorbance and PA signals at visible wavelengths, PA-specific hardware was used to detect the drug in a variety of experimental and clinically-relevant platforms and tested their MΦ-targeting and anti-inflammatory capabilities in a relevant animal model.

### Screening FDA-Approved Anti-Inflammatory Drugs for Candidate PA Imaging Contrast Agents.

Currently, there are 1602 FDA-approved small molecule drugs on the market catalogued in the Drug Bank (Law, V. et al. Nucleic Acids Res. 42, 1091-1097 (2014)). These drugs were sorted to focus on those with clinically established anti-inflammatory activity (126 in number). In order to screen them for intrinsic color with an optical absorption spectrum in the visible color range or near IR range, these short-listed drug molecules were assayed using a combination of optical photography, color identification, literature survey and high-throughput absorption spectrophotometry. While the majority of the compounds were colorless, eight drugs were selected for further testing based on their optical properties while two other molecules were identified to be colored via a parallel literature search. Four of these 10 drugs showed high absorbance at wavelengths (λ)>400 nm (FIG. 11a). Clofazimine (CFZ) exhibited the strongest and most red-shifted absorbance peak wavelength (λₘₐₓ=450 nm). CFZ is an established anti-microbial and anti-inflammatory drug specifically accumulating in MΦs—central regulators of the process of inflammation (Cholo, M. C., et al., J. Antimicrob. Chemother. 67, 290-8 (2012); Baik, J. & Rosania, G. R. PLoS One 7, e47494 (2012); Keswani, R. K. et al. Mol. Pharm. 12, 2528-2536 (2015); Conalty, M. L. & Jackson, R. D. Br. J. Exp. Pathol. 43, 650-654 (1962)).

### PA Spectroscopy of CFZ

A PA spectroscopy unit was used to quantitatively establish the spectral properties of CFZ's PA signal (FIG. 11b). The measurements revealed wavelength-dependent variations in ultrasound wave generation corresponding to shifts in the absorbance spectra of CFZ when protonated (FIG. 11c, 15). As a weakly basic drug possessing two protonatable amine groups, CFZ exhibited a pH-dependent, bathochromic shift of the PA spectra with maxima at λₘₐₓ=450 nm, 495 nm and 540 nm. These three peaks in PA signal corresponded to the absorbance spectra of the free-base, monoprotonated and diprotonated molecular species, respectively (O'Connor, R., et al., Drug Metab. Rev. 27, 591-614 (1995)).

### MΦ-Targeting Characteristics of CFZ

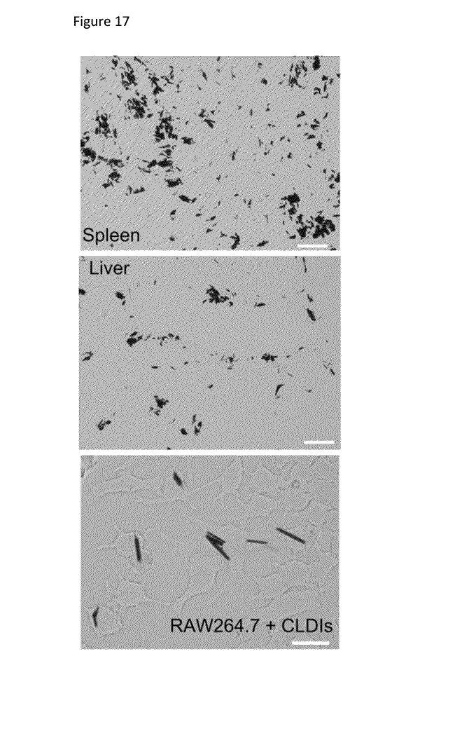

Many animal models to study the clinical pharmacology and pharmacokinetic properties of CFZ have been previously established (Cholo et al., 2012, supra; Banerjee, D. K. et al. Am. J. Trop. Med. Hyg. 23, 1110-1115 (1974)). Independently, to confirm the MΦ-associated accumulation of CFZ, immunofluorescence analyses were conducted using a positive MΦ marker (CD68) (Zhang, X., et al., Curr. Protoc. Immunol. 9, 1-18 (2008)) on histological sections of the spleen and liver from 8 week orally fed CFZ mice. Bright red pigmented spots, consistent with the natural color of the drug, were observed in both the organs (FIG. 17), which were also identified to be fluorescent in the far-red wavelength range (Keswani, R. K., et al., Cytom. Part A 87A, 855-867 (2015)).

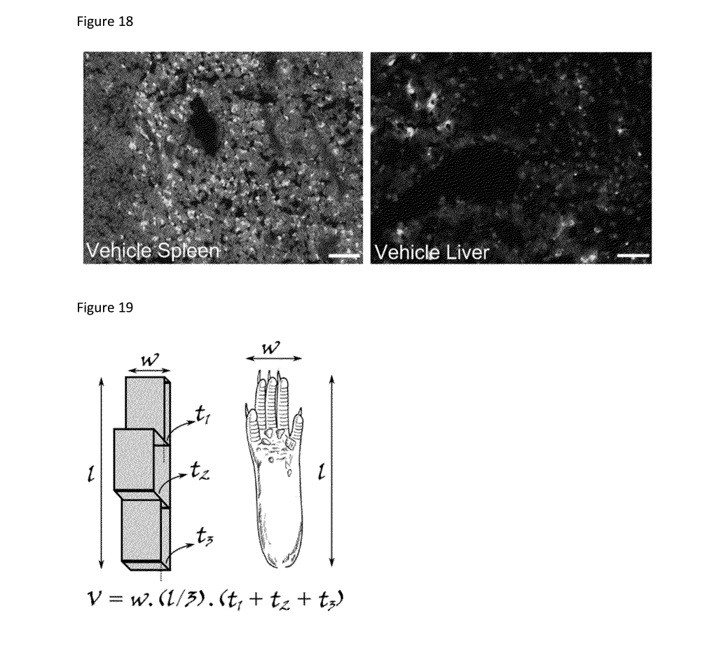

These fluorescent red inclusions co-localised with CD68 expression in both the spleen and liver (FIG. 12a, vehicle-treated cryosections shown in FIG. 18). This observation corroborates previous reports that CFZ does accumulate intracellularly as crystal-like drug inclusions in the MΦs of various organs (Baik et al., 2012, supra; Baik et al., 2013 supra) as reported in many preclinical animal studies as well as in human patients (Cholo et al., 2012, supra; Hastings, R. C., et al., Int J Lepr. Other Mycobact. Dis. 44, 287-293 (1976); Sandler, E. et al., Arch. Pathol. Lab. Med. 116, 541-543 (1992)).

### PA Microscopy of CFZ in MΦs

To quantitatively confirm and establish the PA signal intensity of these MΦ-containing CFZ inclusions, spleen and liver sections of CFZ-treated mice were imaged using a PA microscope using optical excitation at 532 nm (FIG. 12b). PA-responsive regions could be detected in liver (Contrast-to-Noise Ratio (CNR)=22.4 dB, signal intensity=36.0±12.5 mV) and spleen samples of CFZ-treated animals (CNR=24.6 dB, signal intensity=45.9±21.2 mV) (FIG. 12c). Such regions were absent in liver samples of vehicle-treated animals while the spleen sections recorded low PA signal intensity in few areas of the tissue (CNR=23.3 dB, signal intensity=37.4±18.5 mV) (black bars in FIG. 12c). To determine if MΦ associated CFZ inclusions found in the spleen were indeed the source of the PA signal, CFZ inclusions were isolated and purified from the spleen of CFZ treated animals (Yoon, G. S. et al. Mol. Pharm. 12, 2517-2527 (2015)) and incubated with RAW264.7 cells in vitro. Following phagocytosis (Yoon, G. S. et al. Mol. Pharm. 12, 2517-2527 (2015)), high PA signals were again detected in RAW264.7 MΦs containing the ingested CFZ inclusions (CNR=26.7 dB, signal intensity=29.1±14.6 mV) while control RAW264.7 MΦs showed negligible PA signal intensity in the absence of any added CFZ (FIG. 12c).

### PA Imaging of CFZ in Gelatin Phantoms.

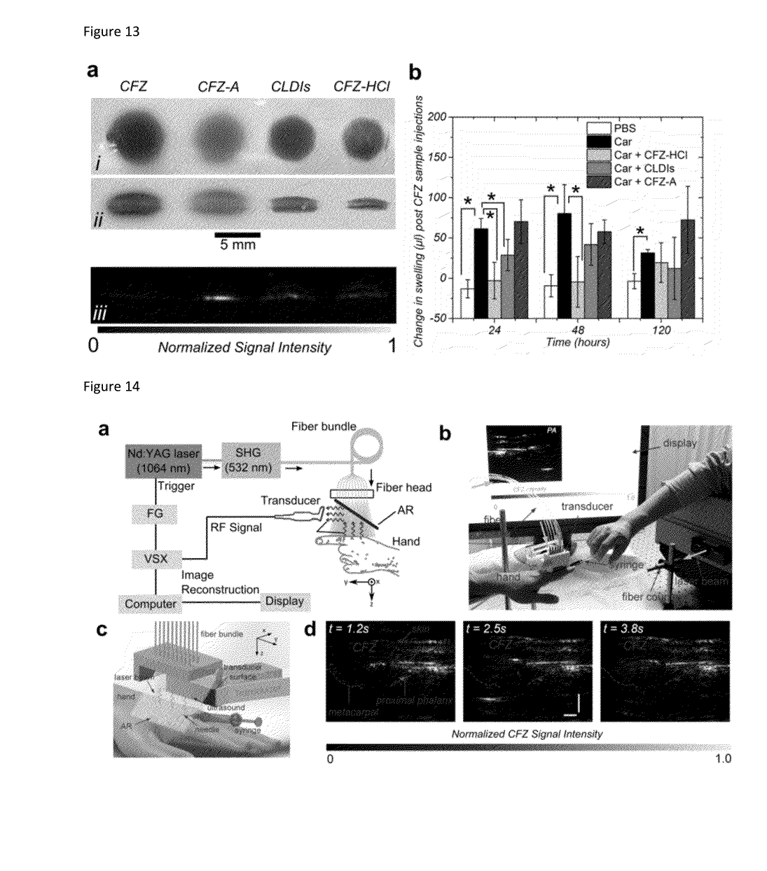

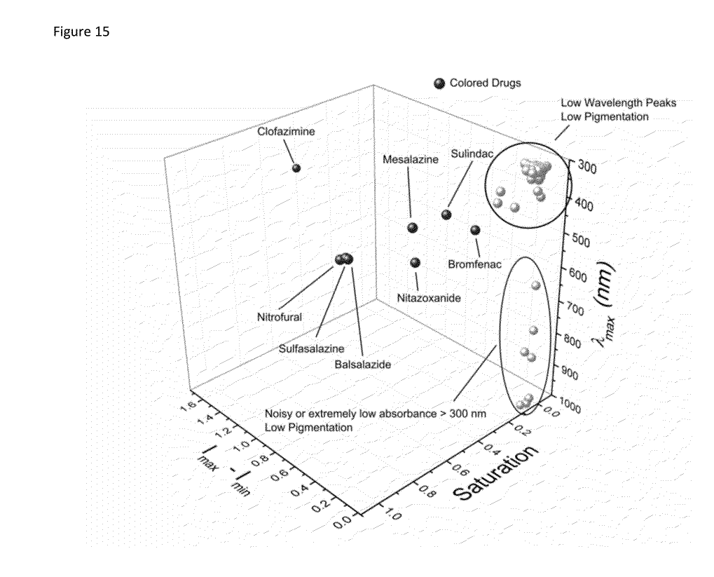

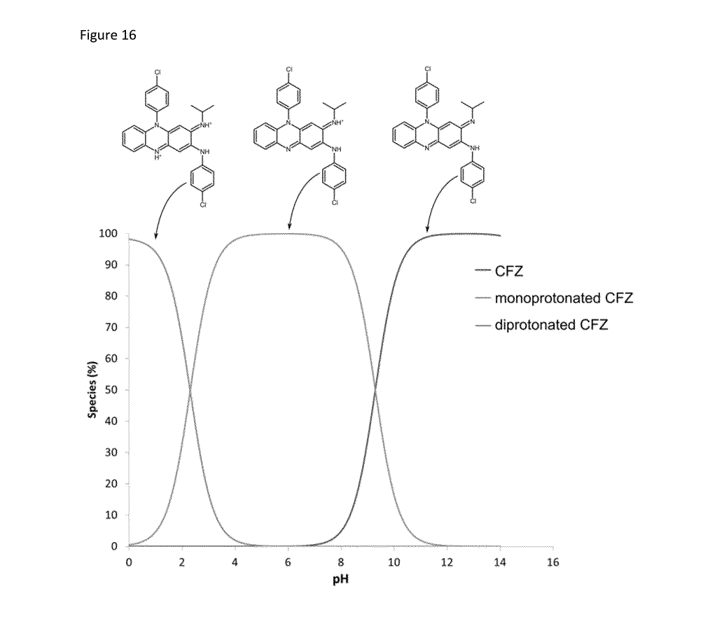

To target the translation from bench to bedside, sufficient imaging depth beneath the sample surface is usually desired. Photoacoustic Tomography (PAT) could offer much better imaging depth beyond the optical mean free path. To establish the suitability of CFZ for PAT, gelatin phantoms were prepared, embedded with sample wells containing soluble CFZ as free-base or in monoprotonated form in DMSO (resolubilized CFZ-H⁺Cl⁻, hereby referenced as CFZ-A), CFZ inclusions isolated from the spleen of drug-treated mice (hereby referred to as Crystal-like-drug-inclusions or CLDIs) in PBS and their synthetic crystal analogue—CFZ-HCl microcrystalline particles in PBS (Keswani, R. K. et al. Mol. Pharm. 12, 2528-2536 (2015)) (FIG. 13a). When visualized with PAT using an excitation wavelength of 532 nm, CFZ-A had a strong PAT signal but CFZ as a free base was minimally detected. Both synthetic CFZ-HCl crystals and mice-derived CLDIs exhibited significant PAT signals.

### Anti-Inflammatory Effect of CFZ

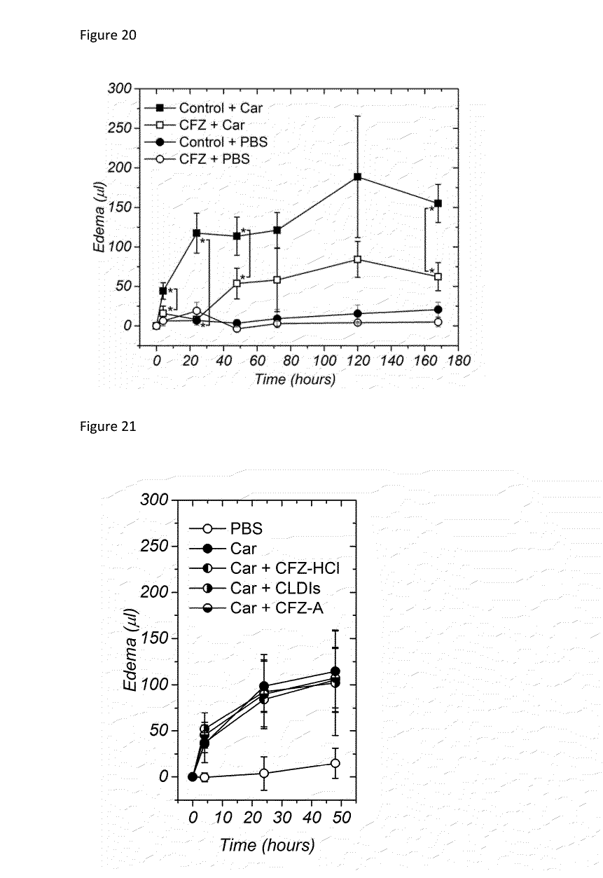

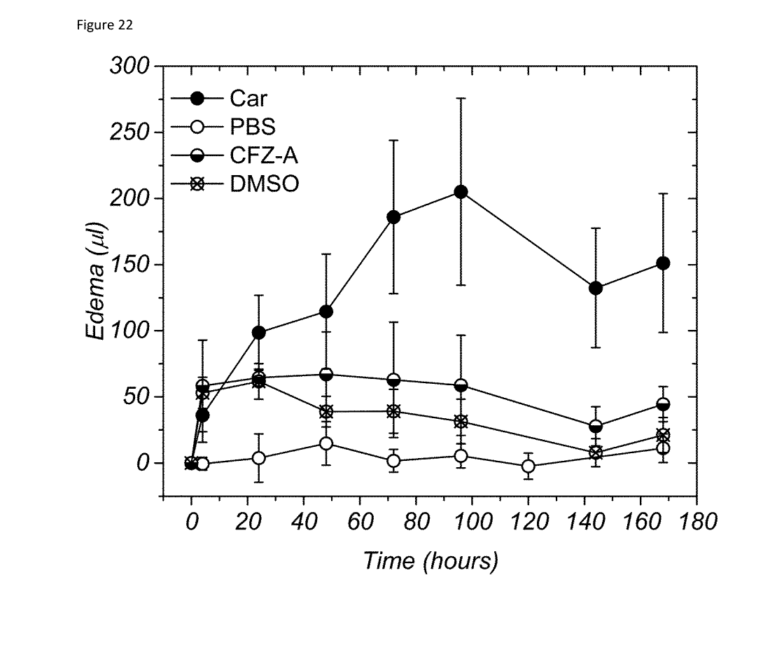

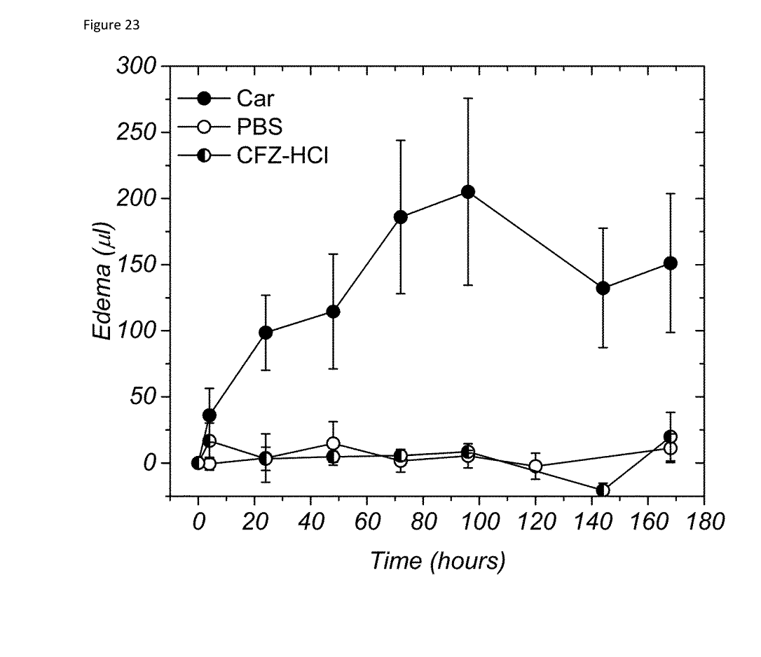

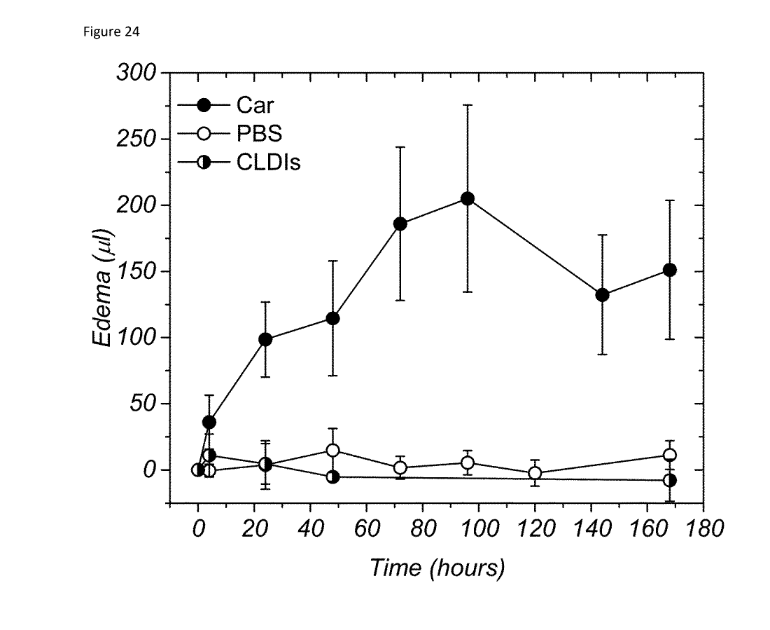

To confirm the in vivo anti-inflammatory activity of PA detectable forms of CFZ, an established carrageenan-based footpad-edema model (Posadas, I. et al. Br. J. Pharmacol. 142, 331-8 (2004)) (swelling measurement model shown in FIG. 19) was used to test whether CFZ had a pro- or anti-inflammatory effect, after administration via different routes. Following 8 week oral treatment with CFZ, mice exhibited decreased inflammation upon stimulation with carrageenan at t=4 (˜3-fold), 24 (˜14-fold) and 48 h (˜2-fold) (*, p<0.05), compared to mice fed with a control drug-free diet (FIG. 20). To test for therapeutic anti-inflammatory activity of local CFZ injections, naïve mice stimulated with carrageenan were re-injected locally in the footpad with CFZ-A, CFZ-HCl or CLDIs at t=48 h post inflammation (FIG. 13b). Prior to re-injection, swelling was equivalent in all the animals (all p>0.1) (FIG. 21). Subsequent swelling in CFZ-HCl and CLDI injected footpads was inhibited for 24 h post therapy while the footpads in the carrageenan-alone treated animals continued to swell (p<0.05) (FIG. 13b). The inhibition was sustained for a further 24 h in CFZ-HCl injected footpads (p<0.05) (FIG. 13b). The anti-inflammatory activity was specific to the CFZ-HCl microcrystalline form of the drug, as injection of CFZ-A did not lead to a measurable anti-inflammatory response (FIG. 13b) (all p>0.1). After 5 days, both treated and untreated animals recovered from their inflammatory injury, with the degree of swelling returning to normal levels (all p>0.1) (FIG. 13b). Injection of CFZ-HCl or CLDIs on their own did not induce any measurable inflammation (FIGS. 22 and 23). In contrast, injection of DMSO alone or in combination with CFZ-A resulted in a measurable inflammatory response initially (up to t=24 h, p<0.05) which declined by t=96 h. (FIG. 24).

### Dual-Modality US-PA Imaging in Human Clinical Model

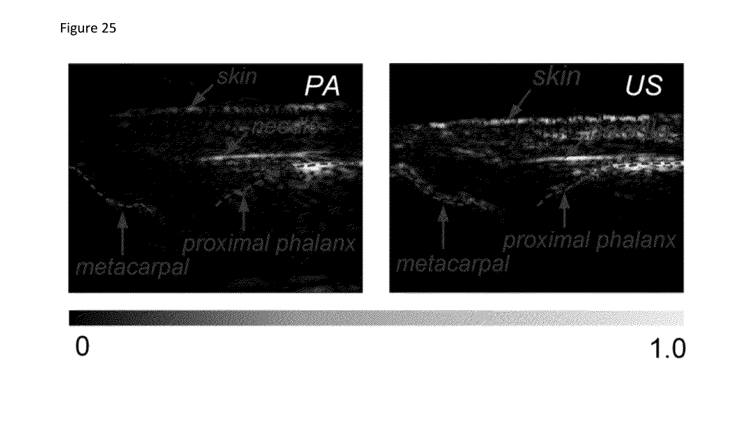

A clinical imaging platform, combining dual modality imaging via PAT and ultrasound (US) (Xu, G. et al. J. Biomed. Opt. 18, 010502 (2013)) (US-PA) was established to determine whether CFZ is useful as a contrast agent for the diagnosis or treatment of arthritis (FIG. 14a,b). The metacarpophalangeal joint (MCP) in the index finger of the hand of a human cadaver (joint usually affected by inflammatory arthritis) was selected as the site of injection of CFZ (FIG. 14c). In comparison with the image taken just before injection (at t=0 s FIG. 25), the images taken at t=1.2 s, 2.5 s and 3.8 s clearly show the appearance and increase of the PAT signal of CFZ in the joint (shown FIG. 14d). While the US shows the structure of the proximal phalanx and the metacarpal forming the MCP joint (FIG. 25), PAT, benefited by the good spatial resolution and high sensitivity to the strong optical absorption of CFZ, shows the time-dependent diffusion of the drug in the joint space.

### Example 3

### Phagocytosed Clofazimine Biocrystals Modulate Innate Immune Signaling by Inhibiting TNFα and Boosting IL-1RA Secretion

### Materials and Methods

### Reagents

All primary antibodies were purchased from Cell Signaling Technology (Danvers, Mass.), except p65 and TLR4 (Abcam, UK), actin and TLR2 (Sigma, St. Louis, Mo.), and TLR9 (Thermo Pierce, Rockford, Ill.). Pam3 and LPS were purchased from Invivogen (San Diego, Calif.).

Purification of CFZ Crystal-Like Drug Inclusions (CLDIs) from Mice Spleen and CFZ Quantification

Clofazimine (Sigma-Aldrich, C8895) was prepared in sesame oil (Shirakiku, Japan, or Roland, China) and Powdered Lab Diet 5001 (PMI International, Inc., St. Louis, Mo.) and orally administrated to C57BL6 mice (4 week old, Jackson Laboratory, Bar Harbor, Me.) for 8 weeks as previously described (Baik et al, 2012, supra; Baik et al., 2013, supra).

Spleens were harvested and CLDIs were isolated using a previously described method with some modifications (Baik et al., 2011, supra). The spleens were cut into small pieces, homogenized with a syringe plunger and then filtered through a 40 μm cell strainer to remove connective tissue debris. The spleen homogenate was centrifuged (300×g for 10 min) to remove large cell debris and the pelleted CLDIs were resuspended in 10% sucrose in Dulbecco's PBS (DPBS) without CaCl₂ and MgCl₂, pH 7.4. CLDIs were further purified using a 3-layer discontinuous gradient (50%, 30% and 10% sucrose in DPBS) centrifugation method (3200×g for 30 min). For incubation with RAW 264.7 cells, CLDIs were washed 3 times with DPBS to remove sucrose and resuspended in DMEM with 5% FBS. Protein concentration of purified CLDI isolates before and after gradient centrifugation and subsequent washing with DPBS was determined using the bicinchoninic acid detection (BCA) assay (Thermo Pierce). CFZ content was spectrophotometrically measured using a previously described method with some modifications (Baik, J. et al., Mol. Pharm. 2011, 8, 1742-1749; Baik et al, 2013, supra). CLDIs in DPBS (100 μl of sample) were mixed with equal volume of xylene to create lipid-aqueous partitioning and then vortexed to dissolve CLDIs and extract the CFZ into the organic phase. The CFZ-containing xylene was placed into a new tube. Fresh xylene was added to the sample and the process was repeated twice until there was no CFZ remaining in the aqueous phase. The CFZ content in xylene was extracted twice using equal volume of 2.5 M H₂SO₄ and vortexing until there was no CFZ remaining in xylene. Final CFZ concentration was calculated from the standard curve generated by adding a known amount of drug solution to 2.5 M H₂SO₄ and measurement of absorbance at 530 nm (Synergy-2 plate reader; Biotek, Winooski, Vt.). The average extraction yield of CFZ was 90%, with elimination of 99% of protein.

Culture of RAW 264.7 Cells with Soluble CFZ or CLDIs and TLR Stimulation

The murine macrophage cell line RAW 264.7 was purchased from ATCC (Manassas, Va.) and maintained in DMEM (Life Technologies, Carlsbad, Calif.) supplemented with 10% FBS. Soluble CFZ or isolated CLDIs were added at various concentrations in DMEM with 5% FBS to 6-well plates containing 4×10⁵ cells/well and incubated (24 h at 37° C. and 5% CO₂). Culture supernatants were harvested at 24 h, centrifuged (1500×g for 5 min), and stored (−20° C.) in frozen aliquots prior to analysis. For experiments involving TLR stimulation, cells were washed twice with pre-warmed DPBS to remove extracellular, non-phagocytosed CLDIs or CFZ, and then the cells were serum-starved for 18 h in DMEM. Supernatants were collected from unstimulated cells and cells stimulated with 200 ng/ml Pam3 or 1 μg/ml LPS in DMEM.

### CLDI Phagocytosis Assay

The ability of RAW 264.7 cells to phagocytose CLDIs was measured by incubating cells with increasing concentrations of CLDIs for 24 h, as described above. The cells were then washed twice with pre-warmed PBS to remove extracellular CLDIs and images were captured using the Nikon Eclipse Ti (Japan) inverted microscope with brightfield to count cells and fluorescence at Cy5 wavelength to count CLDIs. Cells and CLDIs from each image were manually counted to calculate the percentage of CLDI-containing cells and the mean number of CLDIs internalized by each cell. A minimum of 5 random images were analyzed for each concentration (minimum 640 total cells counted). CLDI internalization was confirmed by confocal microscopy, following labeling of the plasma membrane of cells with the lipophilic, fluorescent styryl probe FM-143.

### Live Cell Staining and Imaging

Cells were cultured with CFZ or CLDI media for 24 h in chamber slides (Lab Tek) and stained with 150 nM of MitoTracker Red CMXRos (MTR, Life Technologies) in fresh media for 45 min, and then NucBlue Live Cell Stain (Life Technologies) was applied for 10 min for nuclear staining. After incubation, extracellular MTR and NucBlue were washed twice with DPBS and the cells were visualized on a Nikon Eclipse Ti fluorescence microscope (Japan) using Texas Red (Mitotracker) and DAPI (NucBlue) filters. Real-time live cell images were captured every 10 sec for 30 min with the same microscope and video was created using the Nikon Elements software. For confocal imaging of intracellular CLDIs, FM 1-43 (Life Technologies) was used at 3.5 μM (15 min at 37° C.) for membrane staining followed by NucBlue (10 min). After washing with DPBS, cells were visualized using laser-scanning confocal microscopy (Olympus Fluoview 500) fitted with argon (FITC) and HeNe Red lasers (Cy5). Images were taken along the z-axis with a 60× objective at 0.25 μm intervals and the composite Z-stack images were created using the Nikon Elements software.

### Microscopy and Image Analysis

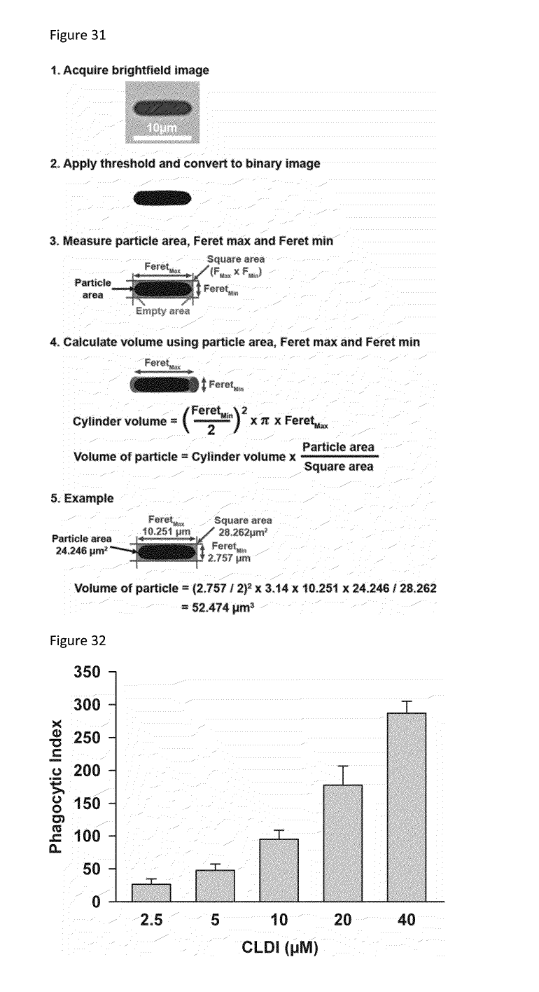

Brightfield and fluorescence (DAPI, FITC, Texas Red and Cy5) images were captured using the Nikon Eclipse Ti (Japan) inverted microscope equipped with a Nikon Digital Sight DS-Fi2 camera (Japan) for brightfield and Photometrics Coolsnap Myo camera (Tucson, Ariz.) for fluorescence. Polarized images were acquired with CRi Abrio Imaging System (Hinds Instruments, Hillsboro, Oreg.) fitted on the same microscope with a 623 nm polarizing filter using the OpenPolScope plugin for ImageJ and Micro-Manager (Mehta, S. B. et al., J. Opt. 2013, 15). For determining the ratio of nucleus-to-cytoplasm p65 fluorescence, ImageJ was used following previously described methods (Fuseler, et al., Microsc. Microanal. 2006, 12, 269-276; Noursadeghi, M. et al., J. Immunol. Methods 2008, 329, 194-200). The average volume of CLDIs was calculated using the area, Feret max and min values of each CLDI acquired from ImageJ. Each CLDI was considered to be cylindrical in shape. A detailed diagram of the calculation method is shown in FIG. 31.

### Cell Viability Assay (XTT Assay)

Cells were plated in triplicate wells at a density of 5×10³ per well in 96-well plates in DMEM with 5% FBS and allowed to adhere overnight. Soluble CFZ (Stock solution 5 mM in DMSO) or CLDIs were added (0.25, 0.5, 1, 2, 4, 10, 20, 40 and 80 μM final concentrations) to cells and incubated (37° C.) for 24 h. XTT assay (Roche, UK) was carried out according to the manufacturer's instructions with absorbance measured at 450 nm and 690 nm using a Synergy-2 plate reader (Biotek). The cell viability percentage was calculated by comparing absorbance of CFZ and CLDI-treated cells to control (untreated) cells.

### TNFα and IL-1RA ELISA

The media of cells with or without 6 h TLR stimulation by Pam3 or LPS was harvested, and TNFα and IL-1RA levels were measured by ELISA (Duoset, R&D Systems, Minneapolis, Minn.) in duplicate wells according to the manufacturer's instructions. The cytokine concentrations were expressed as picogram per milligram of cell lysate. Experiments were repeated three times and values are expressed as the mean±SD.

### Isolation of Alveolar Macrophages

CFZ- or control chow-fed mice were euthanized by exsanguination while deeply anesthetized by intraperitoneal injection of 300 μl ketamine/xylazine. The trachea was surgically exposed and cannulated with an 18 G needle and the lungs were lavaged by instilling DPBS containing 0.5 mM EDTA in 1 ml aliquots for a total of 6 ml. Approximately 90% of the bronchoalveolar lavage (BAL) was retrieved. BAL was then centrifuged for 10 min at 400×g, 4° C. and resuspended in RPMI 1640 media (Life Technologies). The cells were placed in 12-well culture plate (Corning, Tewksbury, Mass.) and washed with media after 45 min, enabling the isolation of alveolar macrophages by adherence. The cells were imaged in brightfield and lysed in RIPA buffer (Sigma) for Western blot.

### Data Processing and Statistics

All data were expressed as mean±standard deviation. Statistical analysis was performed with one-way analysis of variance (ANOVA) and Bonferroni's post-hoc comparisons, or with Student's t-test (paired, two-way). Correlation analyses were performed using a Pearson's correlation coefficient measurement. All statistical analyses employed the IBM SPSS software and p≦0.05 was considered statistically significant.

### Results

Clofazimine CLDI Purification from Spleen.

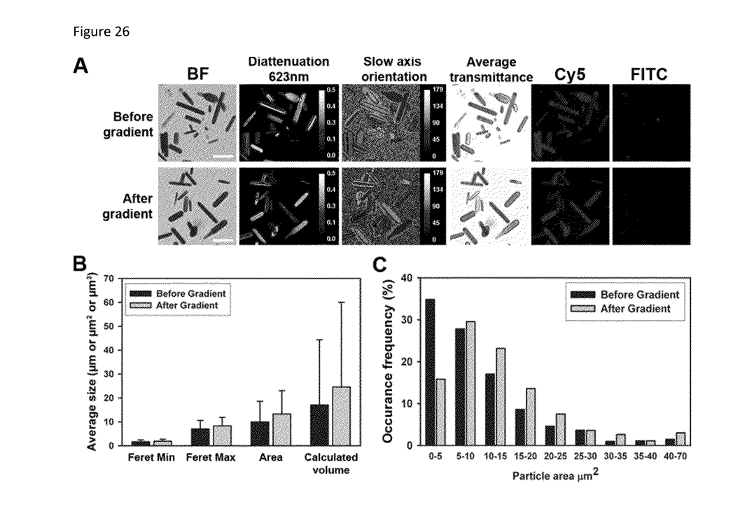

In order to obtain pure CLDIs, a method to isolate and purify CLDIs from the spleen of 8 wk CFZ-fed mice using a 3-layer discontinuous gradient centrifugation method was utilized. Brightfield, polarized and fluorescence microscopy images (FIG. 26A) were acquired before and after gradient centrifugation to characterize and detect any alterations in the physiochemical properties of CLDIs, including crystal structure or chemical composition (Baik et al, 2012, supra). As seen through polarized light microscopy, diattenuation at 623 nm, slow axis orientation and transmittance was maintained in CLDIs after the isolation process, indicative of the preservation of the crystal structure of CLDIs. The CFZ molecule is inherently fluorescent at Texas Red and FITC channels, but the protonation and chlorination of CFZ, as is the case with CLDIs, can cause spectral shifts to Texas Red- and Cy5-positive, but FITC-negative wavelengths (Baik et al, 2012, supra). Therefore, fluorescence microscopy enables the detection of changes in the morphology of CLDIs, as well as changes in the microenvironment surrounding CFZ molecules in CLDIs.

Comparing the optical properties of CLDIs before and after the purification process thus showed that CLDIs were not altered by the purification process. The size dimensions of spleen CLDIs displayed as rod-shaped crystals (FIG. 26A) had an average area of 9.96±8.66 (SD) μm² and Feret dimensions of 7.01±3.59 μm (max) and 1.66±0.74 μm (min) prior to gradient centrifugation (FIG. 26B). The average volume was 17.08±27.27 μm³ before gradient centrifugation. Size analysis showed a heterogeneous population of CLDIs, evidenced by high standard deviations of average area and calculated volume (FIG. 26B), with at least 96% of CLDIs smaller than 30 μm² before gradient centrifugation (FIG. 26C). The purification process removed many of the smaller CLDIs ranging from 0-5 μm², but enriched those over 10 μm² (FIG. 26C), and thus increasing the average area and volume of CLDIs to 13.29±9.73 μm² and 24.57±35.42 μm³, respectively, and Feret dimensions to 8.28±3.57 μm (max) and 1.88±0.82 (min) (FIG. 26B). Most importantly, no degradation, fragmentation, aggregation or other gross morphological changes in the properties of CLDIs were observed during the purification process.

### Active Phagocytosis of CLDIs by RAW 264.7 Cells

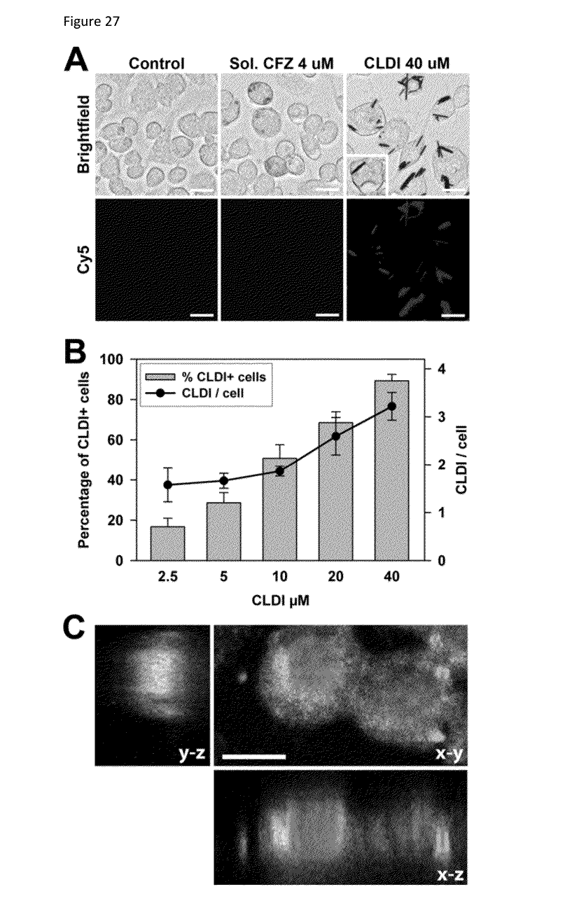

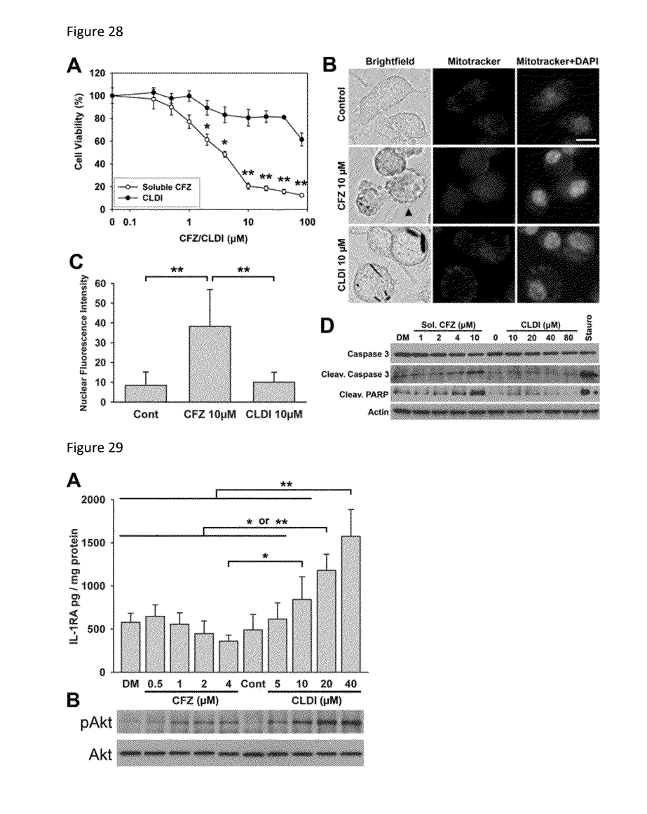

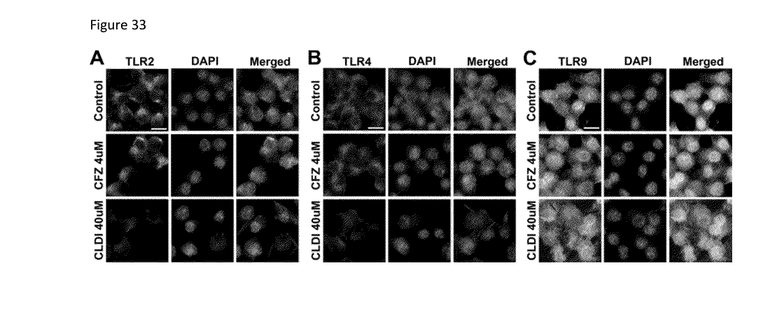

The red color of CFZ allowed monitoring the cellular uptake of soluble CFZ or CLDIs (FIG. 27A). In some of the cells (10-15%), CLDIs were observed inside vacuole-like intracellular vesicles (FIG. 27A inset image). As shown in FIG. 27B, 16.7±4.3% and 28.6±5.1% of the cells showed evidence of CLDI uptake at 2.5 μM and 5 μM concentrations, respectively, without obvious differences in the number of CLDIs per cell (1.58±0.35 and 1.66±0.16). With increasing doses, a dose-dependent increase in the percentage of cells with CLDIs as well as the number of CLDIs per cell was observed. At 40 μM, the maximal dose tested, 89.6±3.22% of cells averaged 3.22±0.29 CLDIs per cell (FIG. 27B). The phagocytic index (percentage of CLDI positive cells×mean number of CLDIs per cell), which measures the ability of macrophages to phagocytose particles (Magenau, A. et al., Traffic 2011, 12, 1730-1743), was highly correlated between CLDI concentration and CLDI phagocytosis (FIG. 32). To confirm that CLDIs were internalized, confocal microscopy and Z-stacking were used to demonstrate the cytoplasmic localization of CLDIs (FIG. 26C). A real-time video of CLDI phagocytosis by a RAW 264.7 cell is shown in FIG. 33. Penetration or damage of the plasma membrane or cell nucleus by CLDIs was not observed.

### Intracellular CLDIs are not Cytotoxic

CLDIs induced minimal cytotoxicity (FIG. 27A) even at very high concentrations (81% viability at 40 but soluble CFZ exerted cytotoxicity at a 20-fold lower concentration (61% viability at 2 μM). Exposure of RAW 264.7 cells to soluble CFZ at 10 μM reduced cell viability to 20%, whereas CLDIs at the same concentration maintained cell viability at 80% (FIG. 27A). Intracellular CLDIs also did not affect mitochondrial membrane potential (FIG. 27B), as seen from the staining of the membrane potential-sensitive (Mitotracker Red; MTR) fluorescent probe. However, macrophages treated with soluble CFZ exhibited diffuse cytoplasmic MTR staining, indicative of mitochondrial membrane depolarization, and displayed fewer mitochondria labeled with MTR. Morphological evidence of apoptosis, such as membrane blebbing, was only seen in soluble CFZ-treated cells (FIG. 27B, arrowhead). MTR signal intensity of the nuclear area was measured to quantify MTR leakage, which was similar in CLDI-treated cells and untreated cells, whereas soluble CFZ-treated cells displayed increased nuclear MTR signal by 4-fold (FIG. 27C).

Changes in mitochondrial membrane permeability such as those leading to difference in MTR staining are upstream of activation of apoptosis pathways mediated by activation of caspases, proteolysis of caspase substrates, and ultimately leading to cell death. Since previous studies also indicated that soluble CFZ could activate apoptosis pathways in cultured macrophages (Magenau, A. et al., Traffic 2011, 12, 1730-1743), experiments were conducted to directly establish the activation of apoptotic pathways by monitoring caspase-3 and PARP cleavage after CFZ or CLDI treatment. Indeed, while soluble CFZ caused increased PARP cleavage at low concentrations (4 and 10 μM; FIG. 27D), CLDIs did not induce caspase-3 activation or PARP cleavage, even at the highest concentrations (80 μM).

### Intracellular CLDIs Activate the Akt Pathway and Enhance IL-1RA Production

Previous studies on mice fed with a CFZ-supplemented diet for an 8 week period indicated increased levels of interleukin 1 receptor antagonist (IL-1RA) an endogenous, secreted anti-inflammatory signaling molecule (Baik et al., 2013, supra). Thus, it was established whether CFZ or CLDIs boosted IL-1RA secretion by cultured macrophages. While soluble CFZ did not affect IL-1RA secretion, phagocytosed CLDIs significantly enhanced the production of IL-1RA in a concentration dependent manner (FIG. 29A). Phosphorylation of Akt, which marks the activation of the signal transduction pathway leading to upregulation of IL-1RA expression (Troutman, T. D., et al., Cell Cycle 2012, 11, 3559-3567; Molnarfi, N. et al., J. Immunol. 2006, 178, 446-454; Rehani, K. et al., J. Immunol. 2009, 182, 547-553) also was increased in concentration-dependent manner in cells that phagocytosed CLDIs. Solubilized CFZ minimally affected phosphorylation of Akt with no effect on IL-1RA expression (FIG. 29B).

### Intracellular CLDIs Dampen TLR2- and TLR4-Mediated NF-κB Activation and TNFα Production

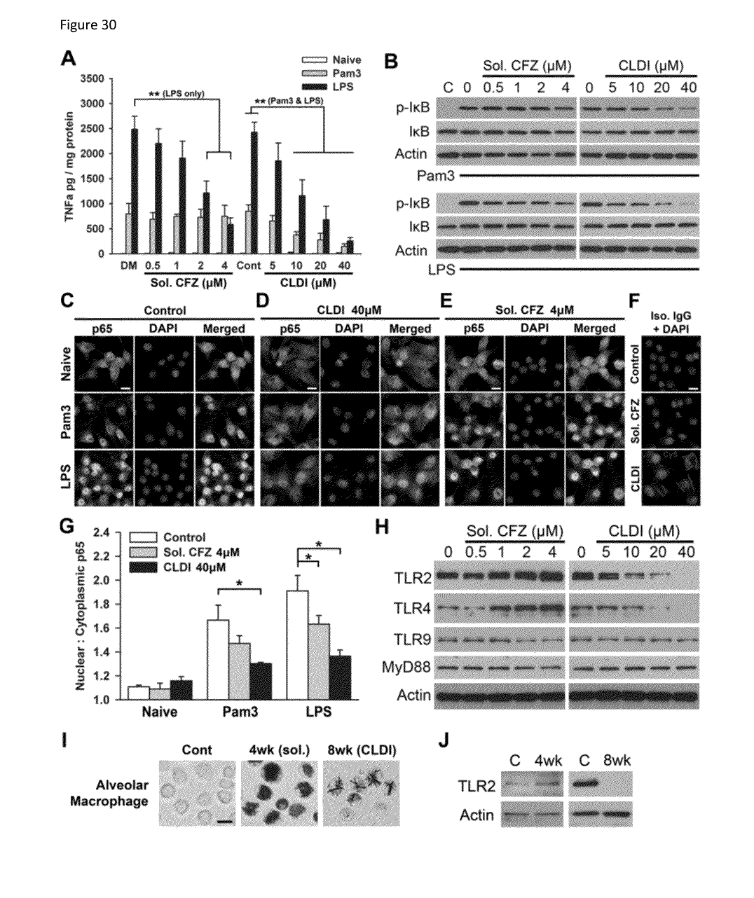

Next, it was tested whether soluble CFZ or CLDIs were able to activate pro-inflammatory signaling pathways by monitoring the secretion of TNFα from the cultured cells. Soluble CFZ or CLDIs failed to induce TNFα production on their own (FIG. 30A). Thus, it was determined if CFZ or CLDIs would promote TNFα release if the cells were pre-stressed with pro-inflammatory stimuli. Both soluble CFZ and CLDIs suppressed TLR2- and 4-mediated inflammatory responses and decreased TNFα production, IκB phosphorylation (p-IκB), and NF-κB (p65) nuclear translocation (FIG. 30 following ligation of TLRs. Ingested CLDIs inhibited TNFα production in response to Pam3 (TLR2) and LPS (TLR4) stimulation at 10 (Pam3, 55.6%; LPS, 52.3%), 20 (Pam3, 67.6%; LPS, 72.1%) and 40 μM (Pam3, 82.9%; LPS, 89.5%) concentrations (FIG. 30A). In contrast, soluble CFZ only inhibited LPS stimulated TNFα at 2 (51.2%) and 4 μM (76.5%) concentrations, while Pam3/TLR2-mediated TNFα production was not affected (FIG. 30A). In order to further understand the mechanisms leading to TNFα inhibition, IκB phosphorylation (FIG. 30B) and NF-κB (p65) nuclear translocation (FIG. 30C-G) was measured. Phosphorylation of IκB leads to the release of bound NF-κB complex, followed by its nuclear translocation and transcription of TNFα and other pro-inflammatory genes. In accordance with TNFα assay results, reduced p-IκB levels were observed in response to Pam3 and LPS stimulation in CLDI-containing cells in a concentration-dependent manner (FIG. 30B). Moreover, soluble CFZ only inhibited LPS/TLR4-mediated IκB phosphorylation, while the Pam3/TLR2-mediated response was not affected. Furthermore, by monitoring the nuclear and cytoplasmic distribution of intracellular NF-κB (p65) in soluble CFZ or CLDI-containing cells with or without Pam3 or LPS stimulation (FIG. 30C-E), and by comparing the ratio of nuclear to cytoplasmic p65 staining intensity, the ability of CLDIs to reduce both LPS and Pam3-induced nuclear translocation of p65 was measured (FIG. 30G). Soluble CFZ (4 μM) reduced p65 translocation in response to LPS, but not Pam3 (FIG. 30G). In addition, the presence of intracellular soluble CFZ or CLDIs alone did not induce nuclear translocation of p65 (FIG. 30G, naïve), and the reduction of p65 nuclear translocation in CLDI-containing cells correlated with the measured reduction in p-IκB levels (FIG. 30B, G).

Inhibition of IκB phosphorylation and NF-κB activation could be achieved by interference with multiple points upstream of the signaling pathway TLR2, TLR4 and TLR9 levels, as well as the adaptor molecule MyD88 were assayed. Increasing concentrations of CLDI treatment led to decreased levels of TLR2 and TLR4 expression following 24 h incubation (FIG. 30H). Conversely, soluble CFZ upregulated TLR2 and TLR4 expression over the same time period. The expression of intracellular TLR9 or MyD88 was not changed in both soluble CFZ and CLDI treated cells. Western blot data were confirmed by immunofluorescence microscopy, which showed downregulation of TLR2 and TLR4, but not TLR9 in CLDI-containing cells (FIG. 33).

In order to verify whether the accumulation of soluble CFZ or CLDIs altered TLR2 expression in mice, primary alveolar macrophages (AM) from mice fed with control chow or CFZ for 4 wks (which are loaded with soluble CFZ but not CLDIs) or 8 wks (which contain CLDIs) (FIG. 30I) were subjected to Western blot detection of TLR2. Relative to untreated controls, TLR2 expression was moderately increased in soluble CFZ-containing AMs obtained from 4 wk treated animals. TLR2 was downregulated in CLDI-containing AMs obtained from 8 wk treated animals (FIG. 30J).

### Example 4

### A Role for Counterion Transport Pathways in Intracellular Drug Disposition

### Methods

Animal Experiments.

Mice (4 week old, male C57Bl6) were purchased from the Jackson Laboratory (Bar Harbor, Me.) and acclimatized for 1 week in a specific-pathogen-free animal facility. Animal care was provided by the University of Michigan's Unit for Laboratory Animal Medicine (ULAM), and the experimental protocol was approved by the Committee on Use and Care of Animals. Clofazimine (CFZ) (C8895; Sigma-Aldrich, St. Louis, Mo.) was dissolved in sesame oil (Roland, China, or Shirakiku, Japan) to achieve a concentration of 3 mg/ml, which was mixed with Powdered Lab Diet 5001 (PMI International, Inc., St. Louis, Mo.) to produce a 0.03% drug to powdered feed (Baik et al., 2013, supra). A corresponding amount of sesame oil was mixed with chow for vehicle treatment. On average, food consumption for a 25 g mouse was 3 g/day, resulting in 10 mg of bioavailable drug/kg per day. For CFZ treatment, the drug diet was carried out for 8 weeks followed by a switch to a control diet for 8 weeks (washout phase).

Measurement of CFZ in Tissues.

CFZ mass was measured as previously reported (Baik et al., 2012, supra; Venkatesan, et al., Arzneim. Forsch. 2007, 57, 472-474). Briefly, at predetermined time points, mice were euthanized using CO₂, and blood was removed through cardiac puncture. Organs and tissues were harvested, washed in cold DPBS, and kept at −20° C. until further analysis. The tissues were homogenized with Tissumizer (Tekmar®, Cincinnati, Ohio) and CFZ was extracted with dichloromethane twice followed by solvent evaporation and resolubilization in methanol (MeOH). CFZ mass was spectrophotometrically determined in methanol (λ=490 nm) and the concentration was calculated using a standard curve generated by spiking extracted tissue of the control (vehicle-only treated) mice tissue with known amounts of drug. The tissue-to-fat partition ratios were computed based on mass of drug in the various tissues relative to mass of drug in total body fat (units: dimensionless; calculated as [mg CFZ/g tissue]/[mg CFZ/g fat]). The tissue to plasma partition ratios were computed based on mass of drug in the various tissues relative to mass of drug in plasma volume (units: g⁻¹ tissue, calculated as [mg CFZ/g tissue]/[mg CFZ in blood]). CFZ mass was measured in relation to the measured weight of the tissue or plasma volume.

Isolation of CLDIs from Mouse Spleens.

Spleen tissue homogenate at 8 weeks post drug feeding was sonicated for 30 minutes and clarified by centrifugation (100×g for 1 min). Supernatant was resuspended in 10% sucrose solution in H₂O followed by centrifugation (100×g) to remove large cell debris. The drug inclusions in the supernatant were then pelleted by centrifugation (21,000×g for 1 min) and resuspended in 20 ml of 10% sucrose. Clofazimine content was spectrophotometrically (k=490 nm) determined using calibrated CFZ standards (Baik et al., 2013; supra; Baik, J. et al., PLoS One 2012, 7, e47494; Baik, J. et al., Mol. Pharm. 2012, 8, 1742-1749).

Preparation and Transmitted Light Microscopy of Cryopreserved Tissues.

After euthanasia, organs removed from CFZ and vehicle fed mice were cryo-preserved in optimal cutting temperature compound (Tissue-Tek 4583; Sakura). Tissue blocks were sectioned at a thickness 10 using a Leica 3050S cryostat. For transmitted microscopy, cryo-sectioned slices were mounted on glass slides with glycerol, and imaged with Olympus X51 upright microscope equipped with X100 objective, DP-70 color camera, and DP controller 3.1.1267.

Sample Preparation and Transmission Electron Microscopy (TEM).

For TEM, mice were euthanized and they were perfused blood-free by infusing 0.1 M Sorensen's buffer into left ventricle. After flushing for five minutes, five times the total blood volume of fixative containing Karnovsky's solution (3% paraformaldehyde, 2.5% glutaraldehyde) was infused. Afterwards, organs were removed, minced, and kept in the fixative solution at 4° C. until further processing. For staining, the tissues were incubated with osmium tetroxide and dehydrated in alcohol. Dehydrated samples were infiltrated with Epon resin and then polymerized at 60° C. for 24 hours. The polymerized tissue blocks were sectioned with an ultramicrotome and post-stained with uranyl acetate and lead citrate. TEM was performed with a Philips CM-100 instrument equipped with a Hamamatsu ORCA-HR camera system operated by Advanced Microscopy Techniques (Danvers, Mass.).

Synthesis of CFZ Crystals.

CFZ was dissolved in MeOH at 2 mM. Equal volumes of anti-solvents were added to obtain the drug crystals—0.1 M HCl—CFZ-A1, 0.1 M NaOH—CFZ-B, H₂O—CFZ-N, 1 M NH₄Cl—CFZ-A2. The supernatant was removed, and the crystals were washed and lyophilized in the dark in preparation for further analysis.

Powder X-Ray Diffraction (p-XRD).

Powder XRD of dried samples of isolated CLDIs, 8 week treated (or vehicle) mouse tissue homogenate and synthetic CFZ samples were carried out with Bruker D8 Advance—Cu K_{α} radiation (λ=1.5406 Å), tube voltage=40 kV, tube current=40 mA. Data were collected at 2θ=4° to 40° at a continuous scan at the rate of 2.5°/min. Diffractograms of the triclinic (DAKXUI01) and monoclinic (DAKXUI) forms of CFZ crystals were imported from Cambridge Structural Database (CSD) and CFZ-TC crystals (C8895; Sigma-Aldrich, St. Louis, Mo.) were used as a positive control for comparison.

Solution NMR.

1D ¹H-NMR and 2D HSQC spectra for various CFZ samples and CLDIs were acquired at the University of Michigan's Biochemical NMR Core Laboratory using an 11.74 Tesla (500 MHz) NMR spectrometer with a VNMRS console and a 7510-AS autosampler system operated by host software VNMRJ 3.2, and equipped with a 5 mm Agilent OneNMR probe with Z-axis gradients. DMSO-d6 (100%, 99.9% atom % D) was purchased from Cambridge Isotope Laboratories, Inc. (Tewksbury, Mass.). The samples were prepared by first freeze-drying the samples overnight followed by dissolution in DMSO-d6 at 2 mg/ml. 128 (¹H) and 8 (HSQC) scans were acquired for each sample. For accurate identification of the NMR peaks in spectrum, a DMSO-d6-solubilized CFZ spectrum was acquired from a saturated solution of CFZ in DMSO: ¹H 64 scans; ¹³C 9000 scans; HMBC 32 scans; HSQC (gc2hsqcse was the pulse sequence) 8 scans; COSY (DQF-COSY) 8 scans; NOESY 8 scans. All the experiments were run at 25° C. and using the standard parameters from VNMRJ4.0 (Agilent Technologies). All the data were processed with MestreNova 9.0 (MestreLab, Santiago de Compostela, Spain).

Bulk Elemental Composition Analysis (BEA).

CFZ samples were pelleted via centrifugation (10,000×g, 2 min) followed by removal of supernatant and freeze-drying overnight. All samples were sent to Atlantic Microlab, Inc. (Norcross, Ga.) for elemental analysis. Established protocols for the measurement of carbon (C), hydrogen (H), nitrogen (N), chlorine (Cl) and sulfur (S) were used to obtain elemental data. All instrumentation used were calibrated daily with ultra-high purity standards. In brief, samples were accurately weighed using electronic microbalances. C, H, N and S analyses were performed on automatic analyzers based on a modified Pregl and Dumas methodology wherein samples were flash combusted in an oxygen atmosphere at ˜1400° C. Quantitative combustion was achieved by passing the mixture of gases over oxidizing agents comprised of copper oxide, EA1000 (chromium and nickel oxide mixture) and tungstic anhydride, and then over copper, maintained at 650° C., to remove excess oxygen and to reduce the oxides of N. The individual components were separated and eluted as CO₂, H₂O, N₂ and SO₂ followed by measurement via a thermal conductivity detector. Cl analysis was performed by Schoniger flask combustion followed by analysis using ion chromatography. The sample was diluted to 25 ml, 50 ml, or 75 ml, filtered and injected into the IC to yield the ppm levels of Cl. All samples were analyzed in duplicate by different technicians.

Secondary Ion Mass Spectroscopy (nanoSIMS).

CFZ-TC and other crystals of CFZ were prepared as mentioned earlier and deposited onto Si wafers and allowed to dry overnight. For CLDI samples, spleens from 8 week CFZ fed mice were isolated and cryo-preserved in optimal cutting temperature compound (Tissue-Tek 4583; Sakura) for histological sectioning as mentioned above. The sections were then scrapped off gently onto a Si wafer. The spatial distribution of various atomic species along the depth of the sample in cross-sectional pattern was mapped with the Cameca NanoSIMS ion micro-probe (CAMECA Instruments, Inc., Madison, Wis., USA). Briefly, with a primary beam of Cs+, focused to a spot-size of 200 nm on the gold-coated surface of the sample, secondary ions of ¹²C, ¹⁶O, ¹⁴N, ²⁸Si, ³²S, ³¹P, ³⁵Cl were sputtered from the sample surface and detected simultaneously in multi-collector mode. Elemental composition was obtained by comparing counts with respect to CFZ-TC as a calibration standard for all atomic species.

X-Ray Crystallography.

Diffraction patterns of CFZ crystals were obtained on a Rigaku Saturn 944+ (Cu K_{α} (λ=1.54178 Å)) at −188° C. Data reduction was performed using CrysAlisPro (Agilent Technologies), and the structure was solved with direct methods using SHELXS²⁷. Structure building, refinement, and electron density map generation were done with SHELXL via the ShelXle GUI (Hübschle et al., J. Appl. Crystallogr. 2011, 44, 1281-1284). Mercury was used to visualize the crystal structure, access CCDC database as well as obtain predicted p-XRD data.

Data Plotting and Statistical Analysis.

All statistical analysis was performed using either ANOVA or Student's t-test. Results were considered significant if p≦0.05. Plots were constructed using Origin 9.0 (OriginLab Corporation, Northampton, Mass., USA) and laid out in figure format using scalable vector graphics format (svg) in either Inkscape (www.inkscape.org) or GIMP (www.gimp.org). NMR plots were constructed in MestreNova 9.0 (MestreLab, Santiago de Compostela, Spain).

### Results

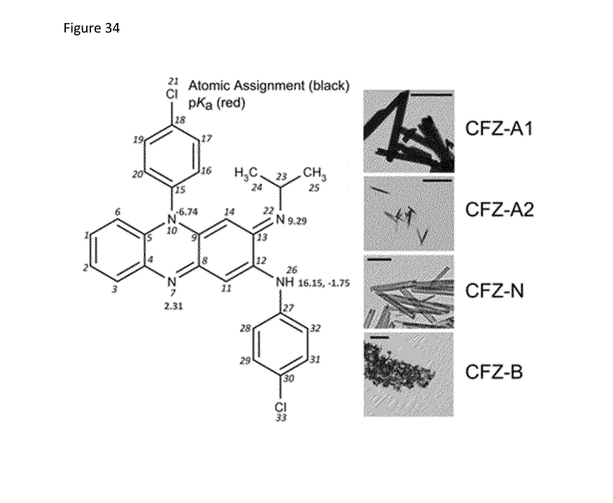

Clofazimine (CFZ)—C₂₇H₂₂Cl₂N₄—is a weakly basic, small molecule chemical agent with a calculated pKₐ₁=2.31 and pKₐ₂=9.29 and is highly lipophilic (clog P˜7). Like other lipophilic weak bases, it has a long retention time within tissues, and exhibits a highly variable pharmacokinetic half-life (t_{1/2}˜70 days in humans, 7 days in mice) (Levy, L. Am. J. Trop. Med. Hyg. 1974, 23, 1097-1109). Upon oral dosing, CFZ exhibits context-dependent pharmacokinetics; while its half-life is in the order of hours to days after an acute dose, upon prolonged oral administration, its half-life is in the order of weeks to months (Levy et al., supra; Banerjee et al., Am. J. Trop. Med. Hyg. 1974, 23, 1110-1115; Nix et al., Tuberculosis 2004, 84, 365-373). In aqueous solution, CFZ predictably exists in neutral, monoprotonated and diprotonated states depending on the solution pH (FIG. 34 shows a graphic of 34) (O'Connor et al., Drug Metab. Rev. 1995, 27, 591-614). Commercially, CFZ is available as a triclinic polymorph of the unprotonated drug (CFZ-TC; Sigma: C8895).

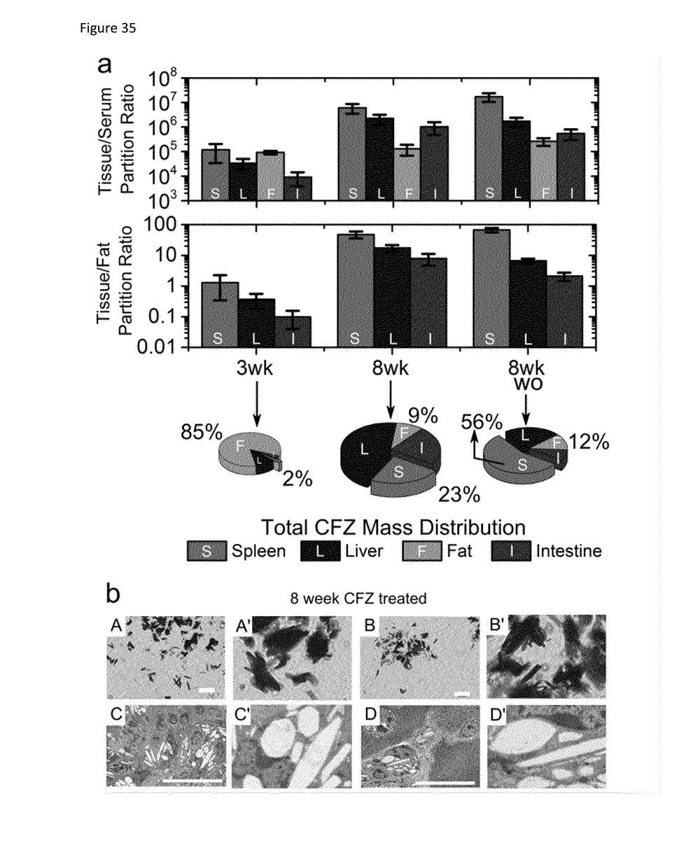

To study the intracellular disposition of CFZ upon prolonged oral dosing, CFZ-TC was fed to 4-5 week old C57Bl/6 mice for at least 8 weeks as an oral diet mixed with sesame oil and regular chow (150 mg, 100 ml, 500 g respectively). Following 3-8 weeks of oral administration, CFZ progressively accumulated within macrophages as membrane-bound crystal-like drug inclusions (CLDIs) (Baik et al., 2012, supra; Baik et al., 2013, supra) in the spleen and liver (FIG. 35 shows 35, 39-40). As expected based on the tendency of hydrophobic small molecules to partition into body fat, CFZ concentration in adipose tissue was >10,000-fold greater than in serum, starting at three weeks (FIG. 35 shows 35a). However, by eight weeks of treatment, tissue-to-serum partition ratios in spleen, liver and intestine dramatically increased and surpassed fat-to-serum partition ratios, relative to three weeks and then remained relatively constant during an eight week washout period (FIG. 35 shows 35a), demonstrating a special sequestration mechanism determining the preferential accumulation and retention of CFZ in these organs relative to body fat. Analyzing the distribution of CFZ revealed that spleen, liver and intestine were major sites of drug retention, after the eight week washout period (FIG. 35 shows 35a). Based on measured partition ratios as well as the mass of drug remaining after the washout period, CFZ mass retained in spleen exceeded its combined mass retained in the other organs (FIG. 35a).

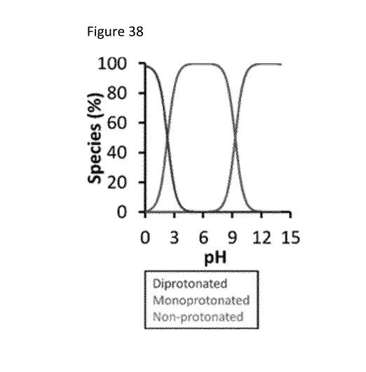

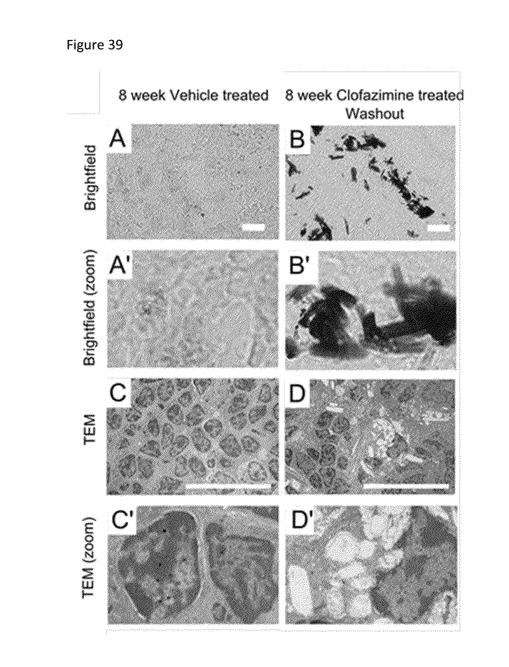

To study the sites of CFZ retention, transmitted light microscopy and transmission electron microscopy (TEM) were used to visualize tissue cryo-sections of spleen and liver (FIG. 35b). CLDIs were observed in the eight week treated mice and even after drug treatment was discontinued (washout phase) in the spleen, liver and intestines (FIGS. 38-39). The intracellular localization and morphology of CLDIs was also independently established using TEM. The spleens of CFZ-treated mice and those of washout animals exhibited cells filled with empty, elongated, and polyhedral CLDI cavities that remained after the drug was extracted during TEM sample preparation process (FIG. 35b). In TEM images, CLDI cavities exhibited faceted, polygonal shapes, with multiple CLDIs clustered within organelle-free cytoplasmic subdomains. In the liver, Kupffer cells were filled with CLDI cavities and arranged in clusters, as previously reported (Baik et al., 2012, supra; Baik et al., 2013, supra). After an eight week washout period, intracellular CLDI cavities were still present within clusters of Kupffer cells (FIG. 35 shows 35b). Overall, other organelles, including endolysosomal vesicles, mitochondria, nuclei, Golgi were generally localized at the periphery of CLDI clusters (Baik et al., 2012, supra). Nuclear morphology of the cells containing CLDIs was normal and similar to that of CLDI-free cells, without any evidence of nuclear condensation or fragmentation which would indicate necrosis or apoptosis.

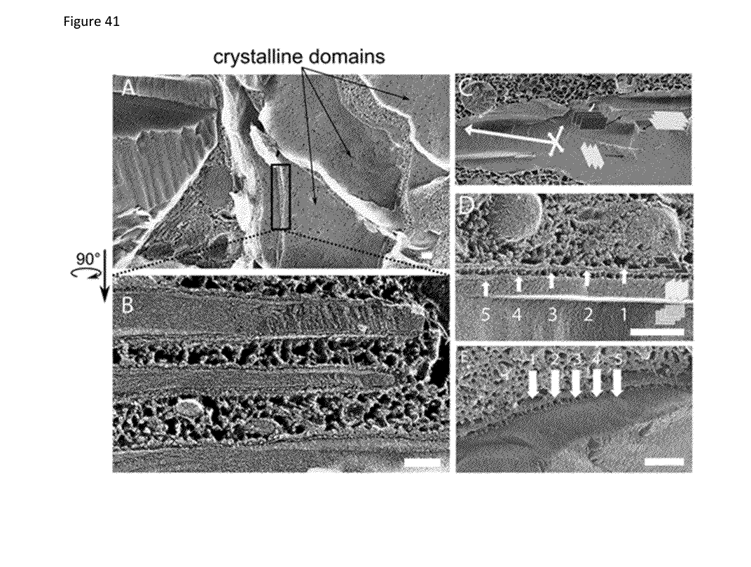

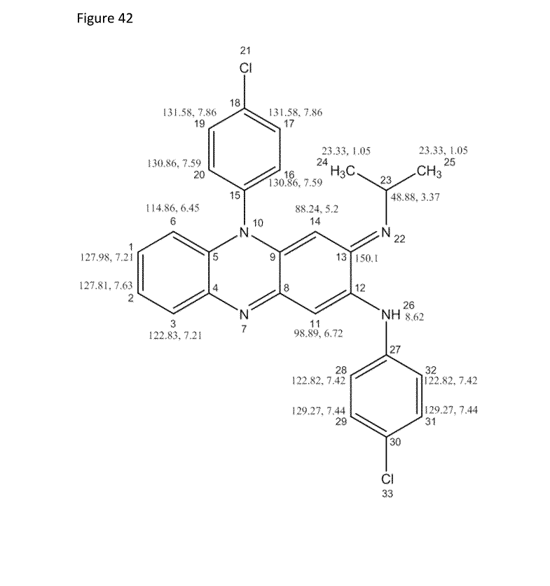

Deep-Etch Freeze-Fracture Electron Microscopy (FFEM) was used to inspect the interface between the internal core of the CLDI and the cytoplasm at high magnification (FIG. 41). It was observed that each CLDI was surrounded by a ˜40 nm thick, multilamellar structure comprised of five distinct layers, with morphological features that were unlike those of any membranes surrounding the other cellular organelles. In direct frontal views of cross-sectional profiles of CLDIs, the membrane layers lacked regular lattice spacings and cleavage planes that characterized the underlying structure of the CLDI cores. Examining this cell-crystal interface revealed a complex arrangement of five distinct membranous layers (FIG. 41, 1 to 5). Also observed surrounding the CLDIs were other organelles including mitochondria, lysosomes, endoplasmic reticulum and nuclei. However, none of the membranes delimiting these other organelles exhibited the characteristic morphological features of the multi-lamellar structure that was consistently observed at the CLDI-cell interface.

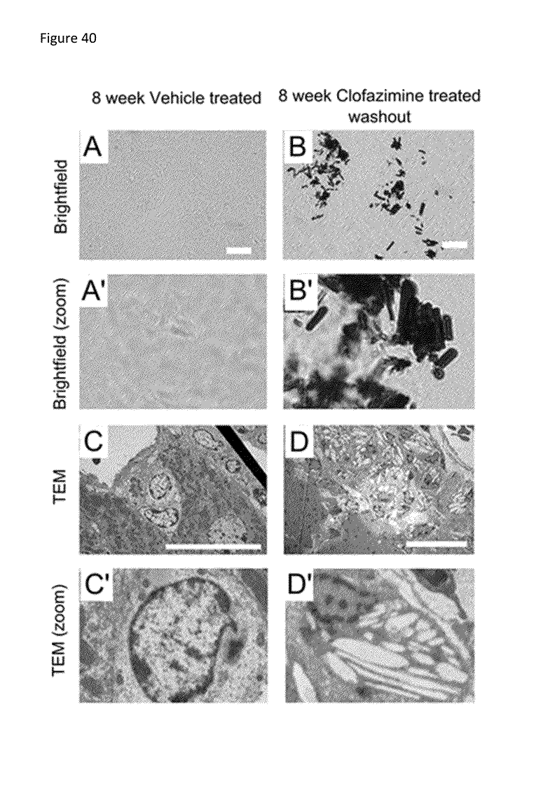

Thus, CLDIs reflect an increase in the partition coefficient of CFZ into these tissues, relative to body fat (FIG. 35 shows 35, FIGS. 39-41). Considering the massive crystallization of the drug, chemical analysis of drug precipitates obtained both biologically as well as through in vitro chemical synthesis was performed. To obtain synthetic drug precipitates, CFZ was recrystallized as different salts through the addition of acidic or basic solvents to pure drug solutions in methanol. The addition of basic (0.1 M NaOH) or neutral (H₂O) anti-solvent led to the formation of orange-colored precipitates (FIG. 34), CFZ-B and CFZ-N, respectively. In contrast, the addition of acidic (0.1 M HCl, pH 0.9-1) or 1 M NH₄Cl (pH 4-5) solutions resulted in dark-red precipitates (FIG. 34 shows a graphic of 34), CFZ-A1 and CFZ-A2 that looked similar to CLDIs. On the other hand, the orange color of CFZ-B and CFZ-N was similar to that of CFZ-TC. To obtain intracellularly crystallized samples, CLDIs were isolated from the eight week CFZ-treated animal spleen via organ rupture followed by centrifugation.

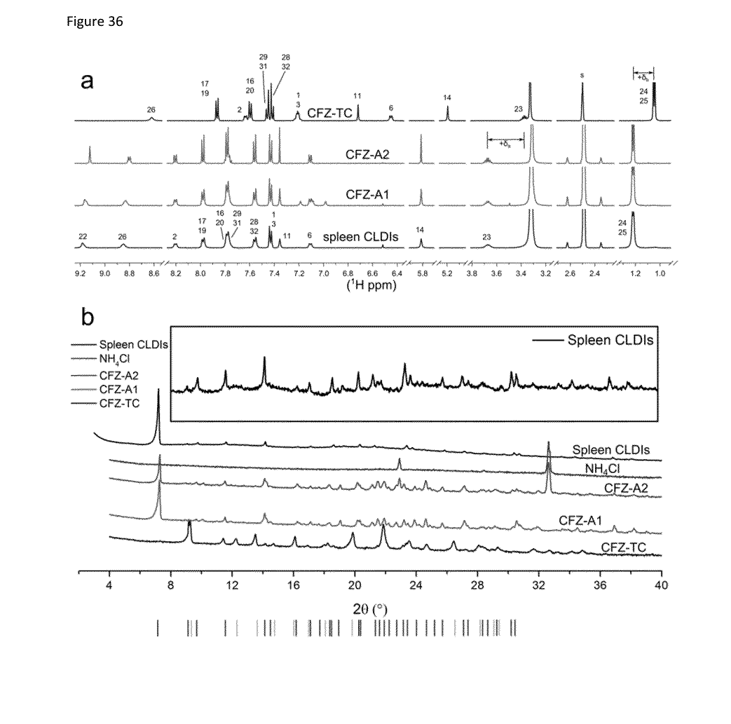

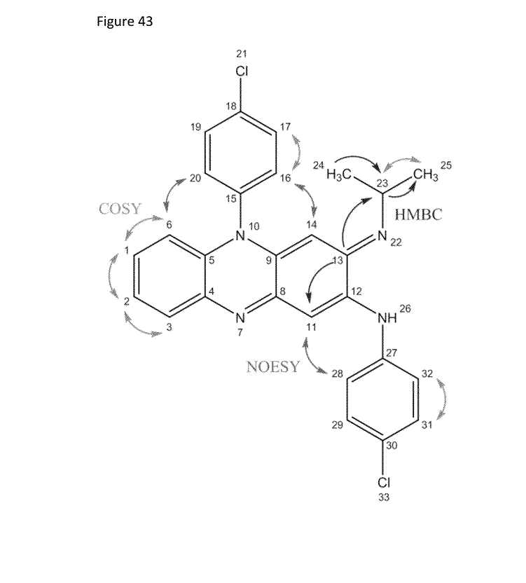

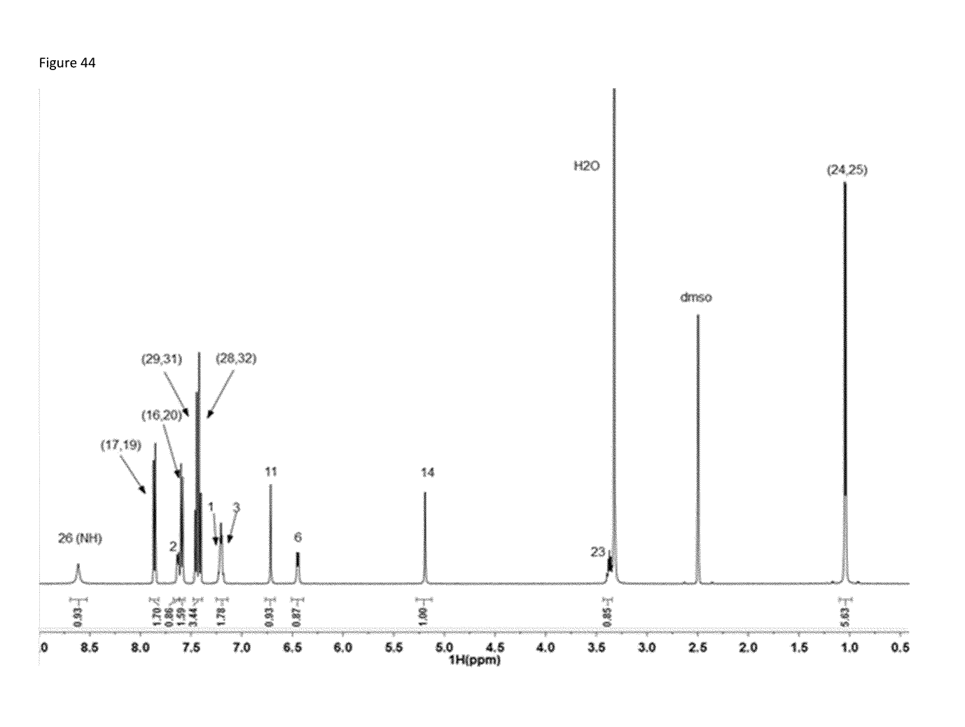

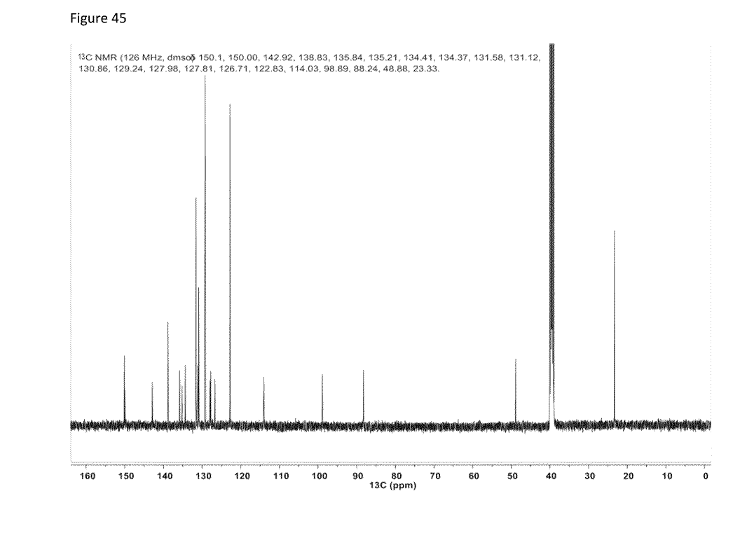

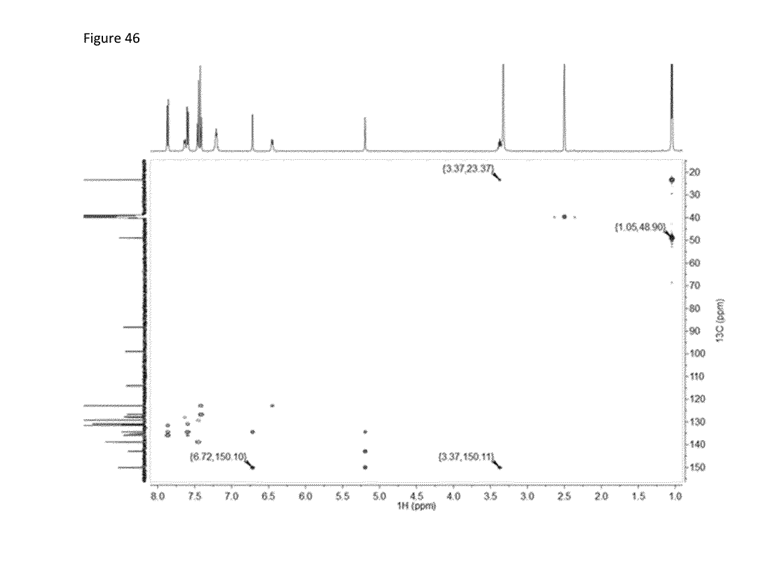

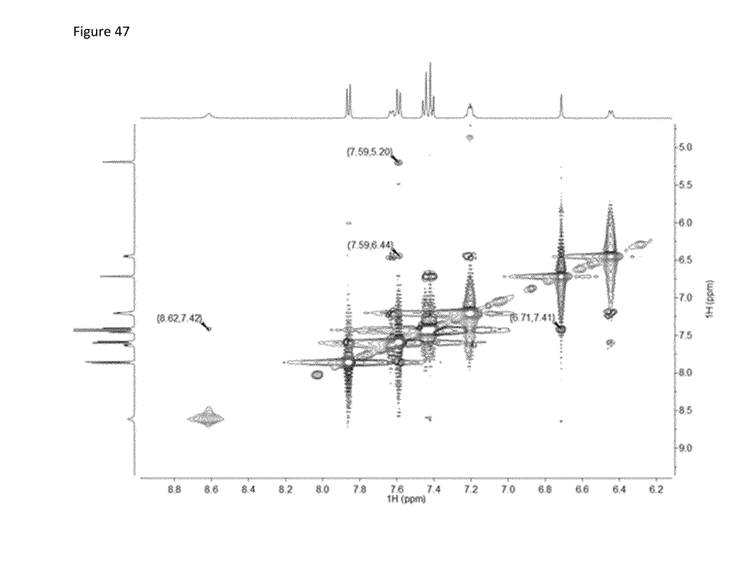

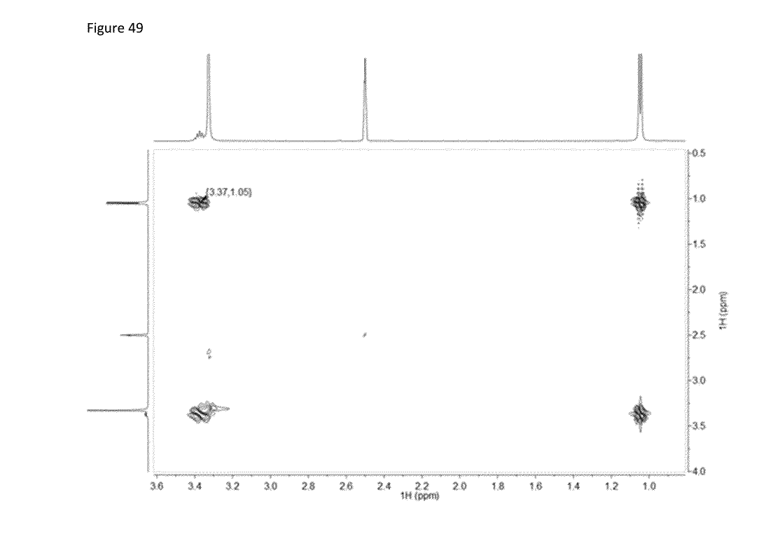

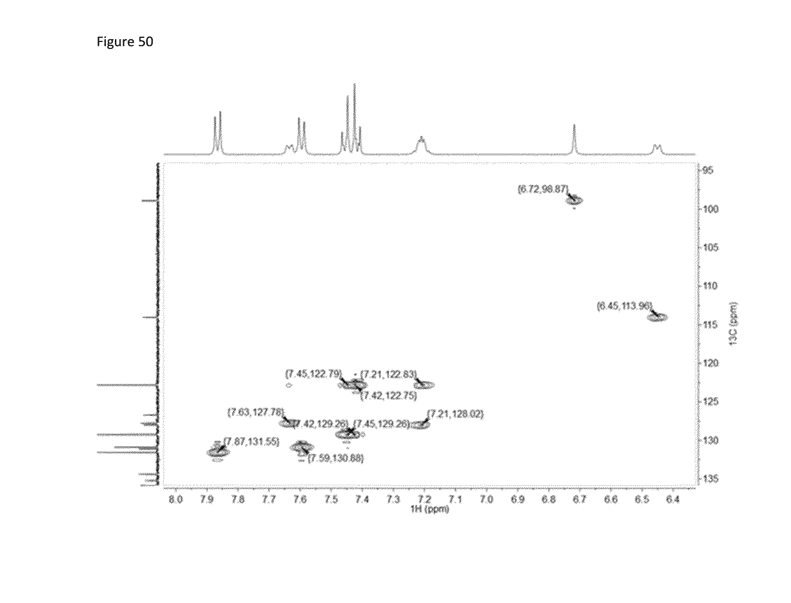

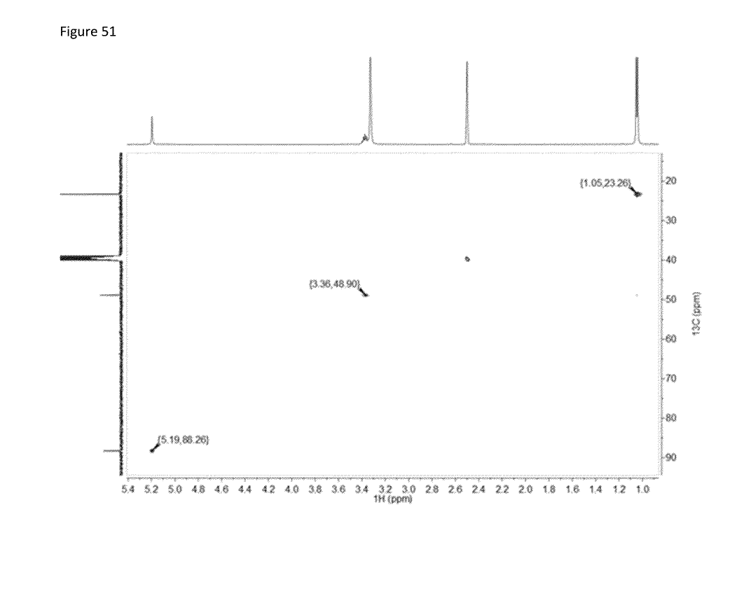

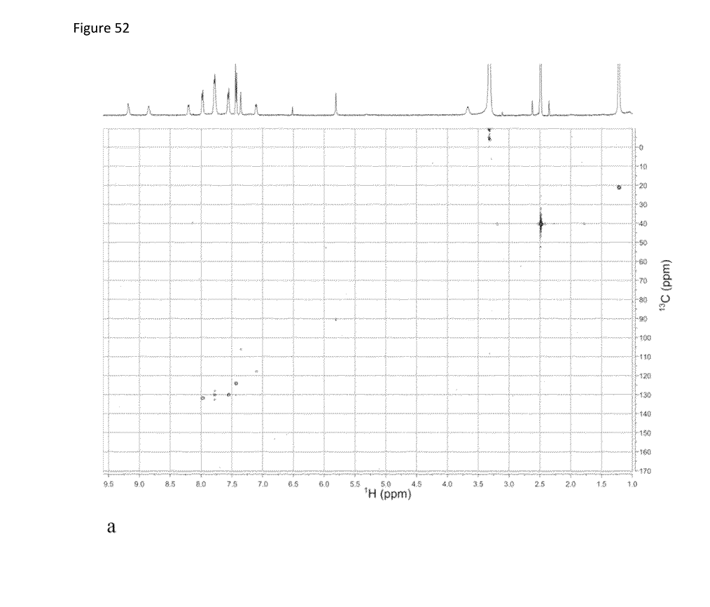

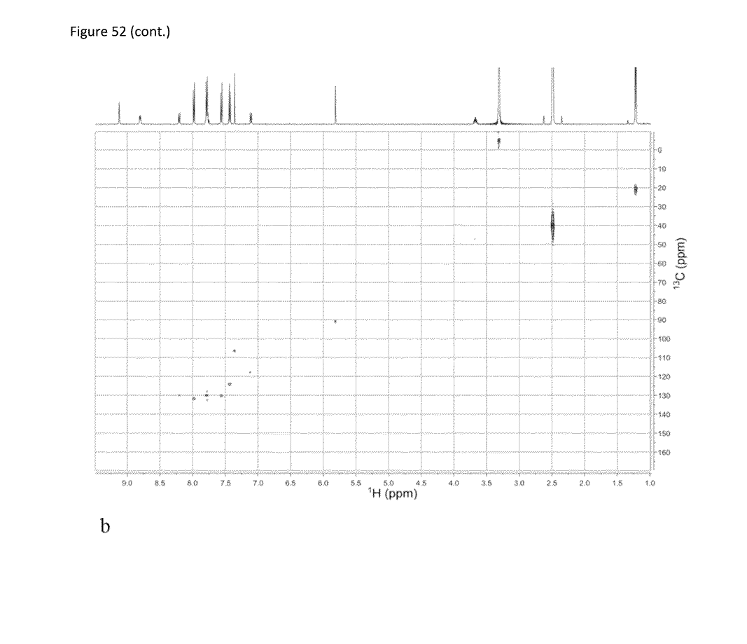

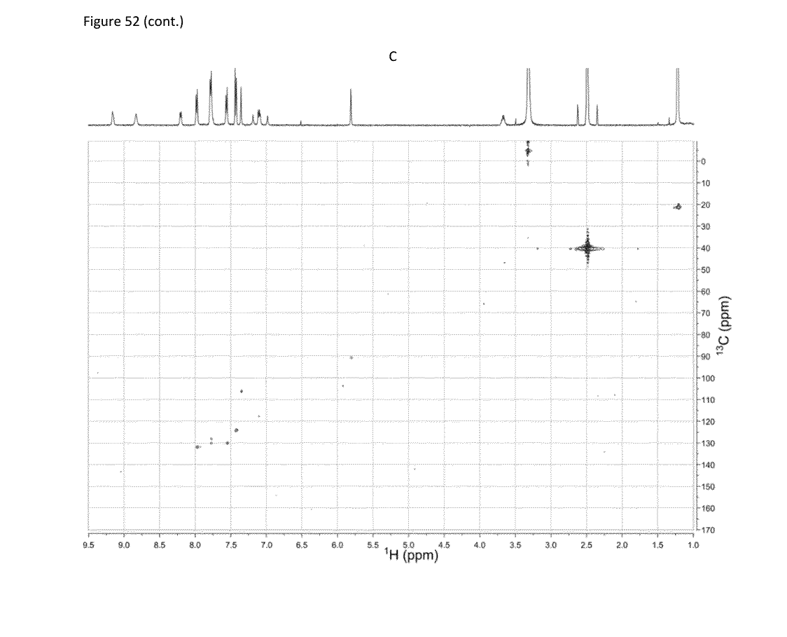

The protonation state of the CFZ molecule in the different CFZ crystals and CLDIs was examined via solution NMR studies, conducted in DMSO-d6. ¹H NMR of CFZ-A1 and CFZ-A2 revealed a chemical shift of +δₐ=0.25 ppm and +δ_{b}=0.15 ppm for the aliphatic protons (1-7, FIG. 34) compared to CFZ-TC, noticeably brought about by the protonation of the amine—22 (pKₐ₂, FIG. 34 shows a graphic of 34, FIG. 36a). The ¹³C, HSQC, HMBC, COSY, NOESY spectra comparing ¹H with their carbons (FIGS. 43-52) and ¹H integration was used to assign NMR peaks to the protons in the molecule. All other protons also exhibited varying chemical shifts (+δ=0.05-0.6 ppm) in CFZ-A1 and CFZ-A2 relative to CFZ-TC. CLDIs isolated from the spleen, CFZ-A1 and CFZ-A2 showed similar ¹H NMR spectra with an additional amine proton (22) that was absent in CFZ-TC (FIG. 36a). Thus, the NMR spectra indicate that CFZ present in CFZ-A1 and CFZ-A2 crystals and, in CLDIs, is monoprotonated.

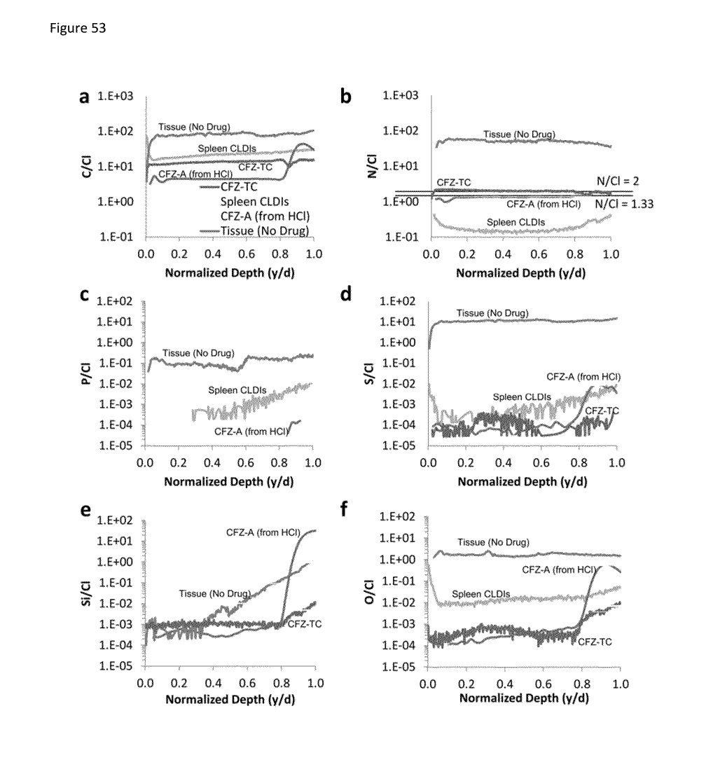

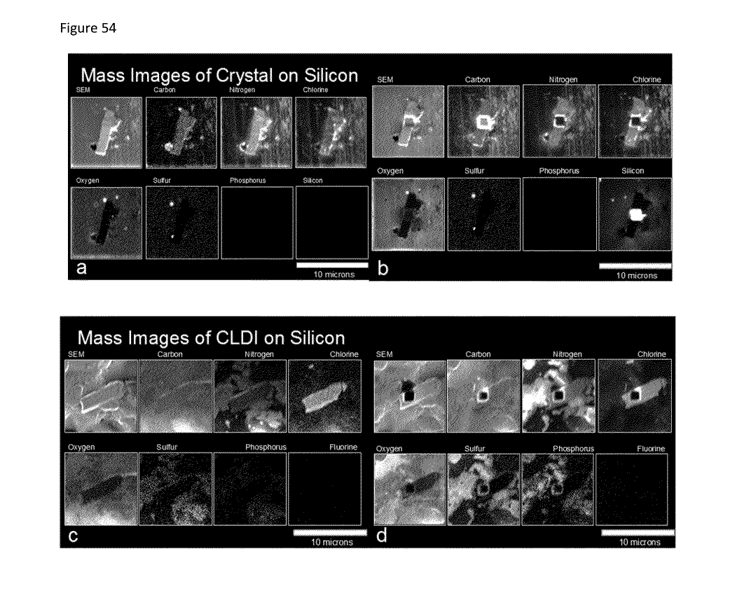

Bulk elemental analysis (BEA) was then performed to determine the exact elemental composition of CFZ-A1, CFZ-A2 and CFZ-TC. Using the ratio N/Cl as a direct indicator of the stoichiometry of Cl in relation to the number of protonatable amines, BEA confirmed the presence of an additional Cl in CFZ-A1 and CFZ-A2 relative to CFZ-TC (CFZ-A1 and CFZ-A2 had an N/Cl=1.33 whereas in CFZ-TC, N/Cl=2; Table 1). This confirmed that CFZ-A1 and CFZ-A2 were identical in chemical composition, confirmed to be the monoprotonated hydrochloride salt of CFZ, hereby referenced as CFZ-HCl. Since, CLDIs were derived from a biological source and contained cellular-derived impurities in the form of membranous domains (FIGS. 39-41), bulk elemental analysis to determine sample stoichiometry would provide an erroneous chemical composition. Hence, CLDIs were analyzed with Secondary Ion Mass Spectroscopy (nanoSIMS) to obtain localized mass distributions within crystals along with other synthetic CFZ crystals. With this instrument, the measured, integrated ion counts are proportional to the elemental composition of the sputtered surface. The counts were plotted in relation to the position of the atom beam to generate a mass image of each isotope in a sample. By progressively scanning a region of a sample, depth profiles of atom counts through the samples were obtained (FIGS. 53-54). To compare atomic composition of CLDIs and reference CFZ crystals, C, N, S, O, P, and Si isotope counts were divided by the corresponding Cl counts and adjusted for differences in detector yield using CFZ-TC as a calibration standard (Table 2). Accordingly, the N/Cl ratio for CFZ-HCl was identical to the value obtained via BEA (=1.33) and lower than CFZ-TC (=2) (Table 1), consistent with the theoretical stoichiometry of Cl per the chemical formula of CFZ-HCl. More remarkably, N/Cl for Spleen CLDIs was 237-fold lower compared to the tissue only sample (no drug) (Table 2, FIGS. 53-54). This is consistent with a high concentration of chloride within CLDIs relative to the rest of the cell. While there was a high concentration of P, S, Si and O in the surrounding tissue and in untreated tissue samples (P/Cl=0.128, S/Cl=0.917, Si/Cl=0.123, 0/Cl=1.704), P, S and Si were undetectable in CLDIs. 0 was marginally present with O/Cl=0.022. The C/Cl ratio demonstrated a much higher accumulation of carbon within CLDIs (23.16) in relation to expected carbon counts based on pure CFZ crystals—CFZ-TC (13.5) and CFZ-A1/A2 (4.43) but much lower than only tissue (81.59). The detection of minor oxygen content and excess carbon in CLDIs confirms the presence of non-drug derived impurities in the form of biological membranes.

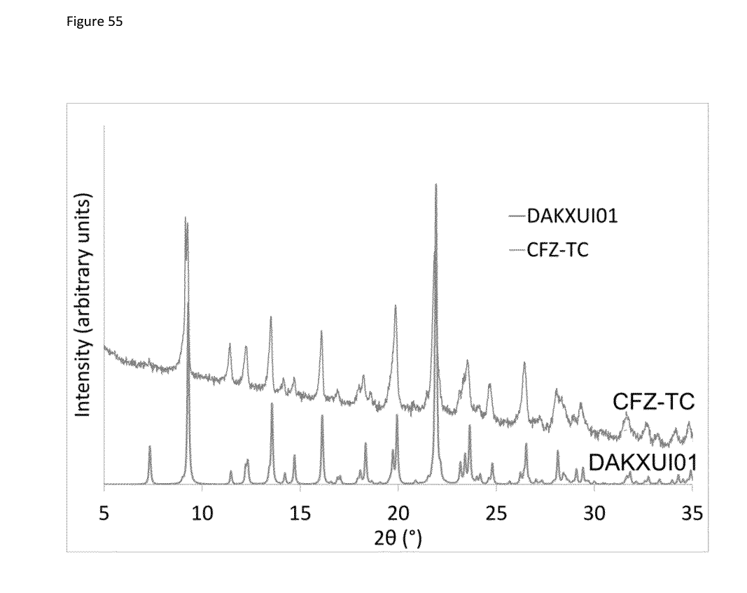

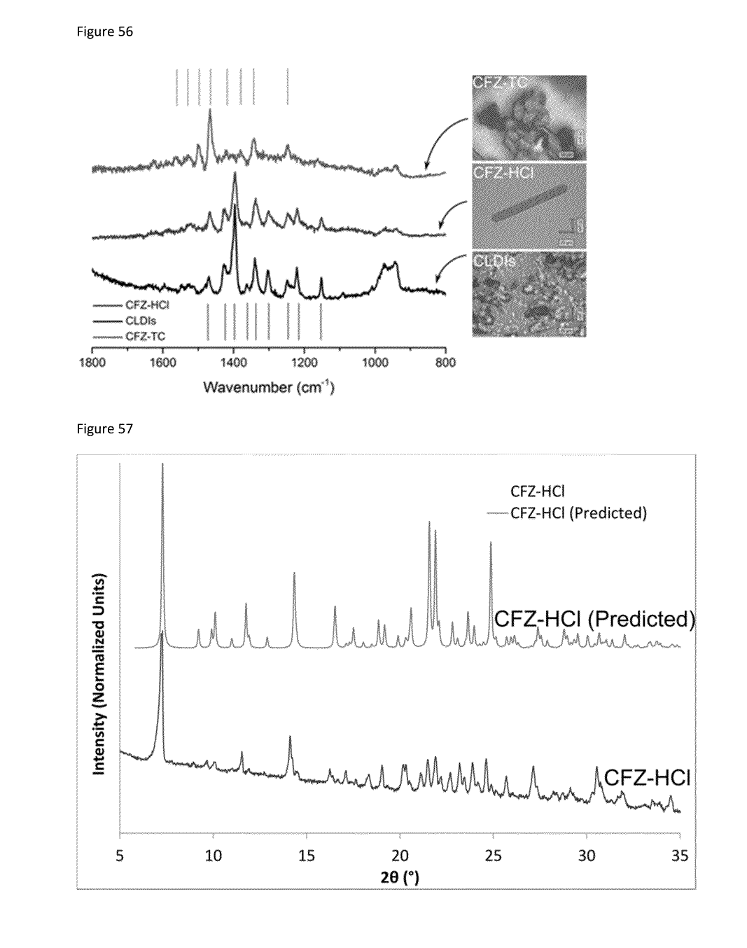

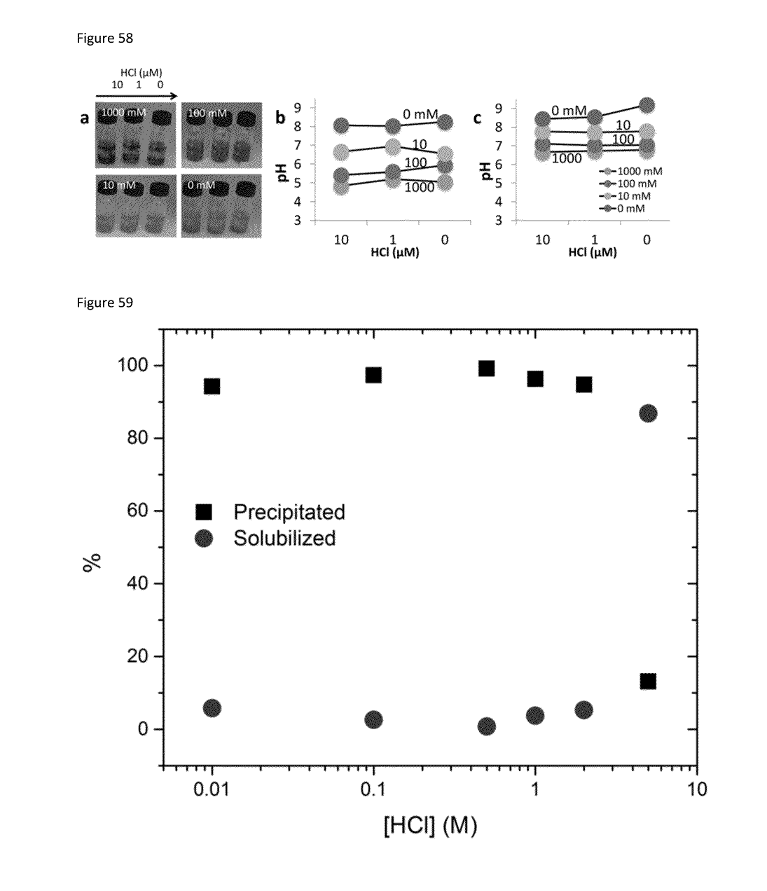

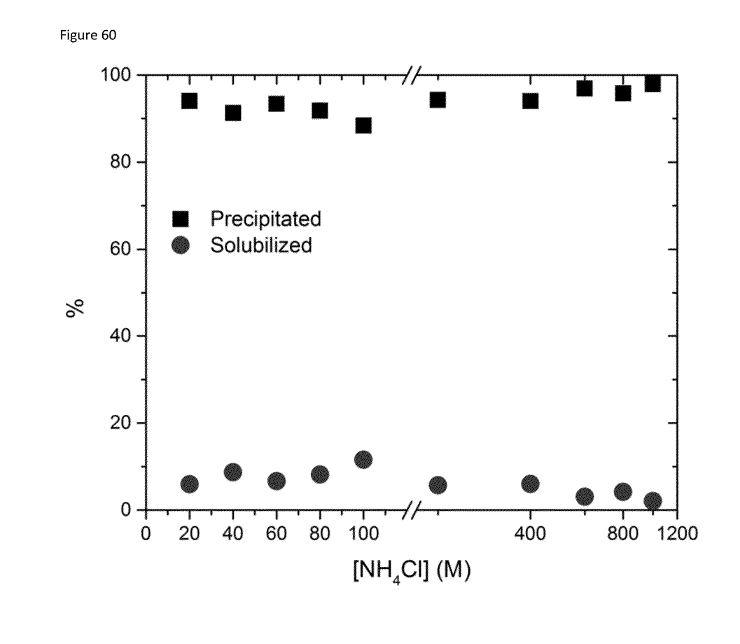

To probe the crystal structures of CLDIs in relation to its different crystal forms, powder X-Ray Diffraction (p-XRD) was performed on samples of isolated spleen CLDIs, CFZ-TC, CFZ-A1 and CFZ-A2. The diffraction peak corresponding to 2κ=9.3° (d=9.71 Å) was present exclusively to CFZ-TC (FIG. 36b, closest crystal analog CCDC refcode—DAKXUI01, FIG. 55) (O'Connor, R. et al., Drug Metab. Rev. 1995, 27, 591-614), while the plane corresponding to 2θ=7.3° (d=12.27 Å) was exclusive to CLDIs, CFZ-A1 and CFZ-A2 (FIG. 36). This single peak was previously reported as being a hallmark of spleen CLDIs (Baik et al., 2012, supra). Hence this particular peak can be considered a signature peak for CFZ-HCl crystals. Additional peaks that were observed in CFZ-A2 (2θ=22.8°, 32.7°) relative to CFZ-A1 were attributable to unreacted, contaminating NH₄Cl crystals. Other prominent peaks were uniquely present in CLDIs, CFZ-A1 and CFZ-A2 (peaks shown in red, FIG. 36b). Also, observed were peaks common to CFZ-TC, CFZ-A1, CFZ-A2 and CLDIs (peaks shown in blue, FIG. 36b; peaks exclusive to CFZ-TC are shown in green). Thus, the p-XRD data clearly indicates that CFZ in CLDIs is present in a structural organization similar to that of pure CFZ-HCl crystals (both CFZ-A1 and CFZ-A2). To further confirm the similarity of the crystallized microstructure of CLDIs relative to CFZ-HCl, Raman microspectroscopy was performed. Peaks observed in CFZ-TC are shown in green while those observed in CFZ-HCl are shown in red (FIG. 55). Consistent with the p-XRD observations, the Raman spectra of CLDIs and CFZ-HCl were similar, exhibiting matching peaks at 1396 cm⁻¹ and 1301 cm⁻¹. These peaks were absent in the Raman spectra of CFZ-TC.

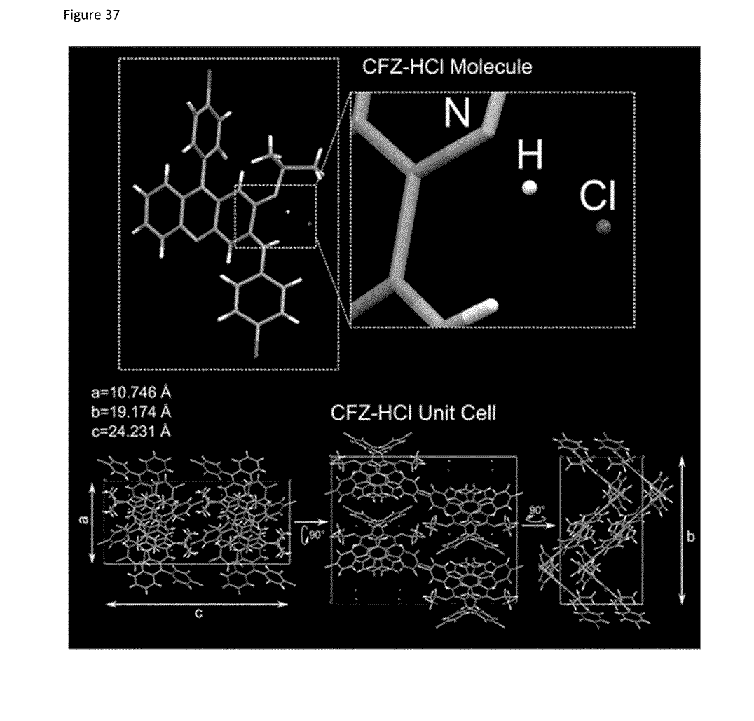

While the X-ray diffraction signals acquired from single CLDIs were not useful in terms of constructing a 3D molecular model, noting the fact that CLDIs and CFZ-HCl are structurally and chemically similar (FIG. 36b), CFZ-HCl crystals were subjected to single crystal analysis. The structure revealed an orthorhombic configuration with 8 molecules/unit cell in CFZ-HCl (submitted to CCDC—Cambridge Crystallographic Data Center, deposition number: 1053818) compared to CFZ-TC (CCDC refcode: DAKXUI01, triclinic, 2 molecules/unit cell) (FIG. 37 shows 37). The stacking of CFZ in CFZ-HCl was remarkably closer than in CFZ-TC with the phenazine backbones being 5.32 Å apart within a dual molecule stack while that in CFZ-TC being 12.15 Å. The phenazine backbone had an inherent torsion of 9.4° in CFZ-HCl and 1.58° in CFZ-TC. The densely packed CFZ-HCl had a calculated density of 1.36 g/ml while CFZ-TC had a density of 1.3 g/ml. The signature peak of CLDIs at 2θ=7.3° (Figure b) was indexed as the 002 plane in the crystal lattice while the signature peak of CFZ-TC at 2θ=9.3° (FIG. 36b) was indexed as the 001 plane in the lattice. Significantly, the predicted p-XRD pattern of the CFZ-HCl based upon its orthorhombic configuration also matched with the absolute p-XRD pattern (FIG. 57). Overall, since the p-XRD pattern of CLDIs match those of CFZ-HCl (FIG. 36b), it can be inferred that CLDIs predominantly contain layers of CFZ as orthorhombic crystalline structures of CFZ-HCl separated by membranes of likely biological origin, consistent with the three dimensional, orthogonal orientation of cleavage faces and fracture plane observed by deep etch-freeze fracture electron microscopy (FIG. 41).

Tables 3-8 show further structural data.

TABLE 1
Bulk Elemental Composition Analysis (BEA) for CFZ—TC,
CFZ—A1, CFZ—A2 (on % impurity-free basis).

| Element | CFZ—TC | CFZ—A1 | CFZ—A2 |
|---|---|---|---|
| C | 68.47 | 63.15 | 63.15 |
| H | 4.82 | 4.66 | 4.66 |
| N | 11.82 | 10.91 | 10.91 |
| Cl | 14.89 | 21.28 | 21.28 |
| S | 0.00 | 0.00 | 0.00 |
| Stoichiometry | CFZ | CFZH + Cl | CFZ + H + Cl |
| N/Cl | 2 | 1.33 | 1.33 |
| (atomic) | | | |

TABLE 2
Elemental ratios through the depth of sputtering obtained
via nanoSIMS. Atomic counts were normalized and calibrated
using CFZ-TC as calibration standard.

| | N/Cl | C/Cl | P/Cl | S/Cl | Si/Cl | O/Cl |
|---|---|---|---|---|---|---|
| CFZ-TC | 2.00 | 13.50 | 0.00 | 0.00 | 0.00 | 0.00 |
| CLDIs | 0.21 | 23.16 | 0.00 | 0.00 | 0.00 | 0.02 |
| CFZ-HCl | 1.33 | 4.43 | 0.00 | 0.00 | 0.00 | 0.00 |
| Tissue only | 50.14 | 81.59 | 0.13 | 0.92 | 0.12 | 1.70 |

TABLE 3
¹H NMR Integrals for (a) CFZ—TC, (b) Spleen CLDIs,
(c) CFZ—A1 and (d) CFZ—A2 in
DMSO-d6. Normalized and Absolute
values obtained via Mestrenova 9.0 software.

| | | | |
|---|---|---|---|
| Range | Normalized | Absolute | Approximate |

(a) Total protons = 22

| | | | |
|---|---|---|---|
| 8.76 . . . 8.49 | 0.87 | 18176.47 | 1 |
| 7.91 . . . 7.83 | 1.8 | 37434.79 | 2 |
| 7.71 . . . 7.55 | 2.75 | 57348.18 | 3 |
| 7.50 . . . 7.33 | 3.73 | 77855.28 | 4 |
| 7.30 . . . 7.12 | 1.89 | 39469.53 | 2 |
| 6.80 . . . 6.65 | 0.91 | 18903.31 | 1 |
| 6.53 . . . 6.38 | 0.92 | 19082.08 | 1 |
| 5.29 . . . 5.12 | 0.97 | 20146.79 | 1 |
| 3.43 . . . 3.34 | 1.12 | 23447 | 1 |
| 1.13 . . . 0.95 | 6 | 125120.87 | 6 |

(b) Total protons = 23

| | | | |
|---|---|---|---|
| 9.53 . . . 9.35 | 0.81 | 17272.83 | 1 |
| 9.17 . . . 8.99 | 0.8 | 17011.21 | 1 |
| 8.27 . . . 8.11 | 0.74 | 15726.07 | 1 |
| 8.05 . . . 7.88 | 1.67 | 35331.51 | 2 |
| 7.86 . . . 7.65 | 3.22 | 68210.8 | 4 |
| 7.63 . . . 7.50 | 1.68 | 35573.18 | 2 |
| 7.48 . . . 7.40 | 1.89 | 40039.91 | 2 |
| 7.38 . . . 7.31 | 0.68 | 14344.55 | 1 |
| 7.15 . . . 7.03 | 0.73 | 15383.8 | 1 |
| 5.89 . . . 5.70 | 0.83 | 17589.38 | 1 |
| 3.76 . . . 3.56 | 0.86 | 18314.81 | 1 |
| 1.39 . . . 0.97 | 6 | 127191.21 | 6 |

(c) Total protons = 23

| | | | |
|---|---|---|---|
| 9.53 . . . 9.35 | 1.33 | 11932.29 | 1 |
| 9.17 . . . 8.99 | 1.4 | 12582.79 | 1 |
| 8.23 . . . 8.18 | 1.08 | 9706.01 | 1 |
| 8.01 . . . 7.95 | 1.9 | 17031.16 | 2 |
| 7.83 . . . 7.73 | 3.8 | 34050.82 | 4 |
| 7.60 . . . 7.53 | 1.9 | 17037.8 | 2 |
| 7.47 . . . 7.40 | 2.06 | 18492.37 | 2 |
| 7.39 . . . 7.33 | 1.11 | 9972.09 | 1 |
| 7.15 . . . 7.06 | 1.38 | 12421.75 | 1 |
| 5.83 . . . 5.79 | 1.08 | 9688.66 | 1 |
| 3.75 . . . 3.60 | 1.81 | 16225.97 | 1 |
| 1.39 . . . 0.97 | 6 | 53835.19 | 6 |

(d) Total protons = 23

| | | | |
|---|---|---|---|
| 9.53 . . . 9.35 | 1.15 | 10338.59 | 1 |
| 9.17 . . . 8.99 | 1.19 | 10682.72 | 1 |
| 8.23 . . . 8.18 | 0.98 | 8772.05 | 1 |
| 8.01 . . . 7.94 | 1.89 | 16972.23 | 2 |
| 7.83 . . . 7.72 | 3.8 | 34099.59 | 4 |
| 7.58 . . . 7.52 | 1.85 | 16619.91 | 2 |
| 7.47 . . . 7.41 | 2.01 | 17987.13 | 2 |
| 7.38 . . . 7.33 | 1.08 | 9671.11 | 1 |
| 7.14 . . . 7.06 | 1.62 | 14533.35 | 1 |
| 5.86 . . . 5.76 | 1.34 | 11976.44 | 1 |
| 3.75 . . . 3.60 | 1.51 | 13560.22 | 1 |
| 1.39 . . . 0.97 | 6 | 53780.85 | 6 |

TABLE 4
Peaks from XRD measurements (2θ = 0-30°)

| | | | 4 | 5 |
|---|---|---|---|---|
| 1 | 2 | 3 | Spleen | CFZ—HCl- |
| DAKXUI01 | CFZ—TC | CFZ—HCl | CLDIs | Predicted |
| 7.3^{~} | | 7.3 | 7.3 | 7.3^{~} |
| | | 9.1 | 9.1 | 9.1^{~} |
| 9.3^{#} | 9.3 | | | |
| | | 9.5 | 9.5 | 9.5^{~} |
| | | 10.0^{#} | | 10.0^{~} |
| | | | | 11.0 |
| 11.5\hat{ } | 11.5 | 11.5 | 11.5 | 11.5\hat{ } |
| 12.2^{#} | 12.2 | | | |
| | | | | 12.9 |
| 13.5^{#} | 13.5 | | | |
| 14.0\hat{ } | 14.0 | 14.0 | 14.0 | 14.0\hat{ } |
| | | 14.5* | 14.5* | |
| 14.7^{#} | 14.7 | | | |
| 16.1^{#} | 16.1 | | | |
| | | 16.2 | 16.2 | 16.2^{~} |
| 16.8^{#} | 16.8 | | | 16.8^{#} |
| | | 16.9 | 16.9 | 16.9^{~} |
| | | | | 17.2 |
| | | 17.6 | 17.6 | 17.6^{~} |
| 18.0^{#} | 18.0 | | | |
| 18.2\hat{ } | 18.2 | 18.2 | | 18.2\hat{ } |
| 18.6\hat{ } | 18.6 | | 18.6 | 18.6\hat{ } |
| | | 18.9 | 18.9 | 18.9^{~} |
| | | | | 19.2 |
| 19.7 | | | | |
| 19.9^{#} | 19.9 | | | 19.9^{#} |
| | | 20.2 | 20.2 | 20.2^{~} |
| | | 20.4 | 20.4 | 20.4^{~} |
| | | 21.0* | 21.0* | |
| | | 21.5 | 21.5 | 21.5^{~} |
| 21.9\hat{ } | 21.9 | 21.9 | 21.9 | 21.9\hat{ } |
| | | 22.2 | 22.2 | 22.2^{~} |
| | | 22.7 | 22.7 | 22.7^{~} |
| 23.2\hat{ } | 23.2 | 23.2 | 23.2 | 23.2\hat{ } |
| 23.5\hat{ } | 23.5 | 23.5 | 23.5 | 23.5\hat{ } |
| 24.0\hat{ } | 24.0 | 24.0 | 24.0 | 24.0\hat{ } |
| 24.5\hat{ } | 24.5 | 24.5^{#} | | 24.5\hat{ } |
| | | 25.2 | 25.2 | 25.2^{~} |
| | | 25.8 | 25.8 | 25.8^{~} |
| | | | | 25.9 |
| 26.0 | | | | |
| | | | | 26.1 |
| 26.5^{#} | 26.5 | | | |
| | | 27.1 | 27.1 | 27.1^{~} |
| 27.2\hat{ } | 27.2 | 27.2 | 27.2 | 27.2\hat{ } |
| 28.1^{#} | 28.1 | | | |
| 28.3\hat{ } | 28.3 | 28.3* | 28.3* | |
| | | 28.7 | 28.7 | 28.7^{~} |
| 28.9^{#} | 28.9 | | | |
| | | 29.0 | 29.0 | 29.0^{~} |
| 29.3^{#} | 29.3 | | | |
| | | | | 29.5 |
| | | | | 30.0 |
| | | 30.2 | 30.2 | 30.2^{~} |
| | | 30.5 | 30.5 | 30.5^{~} |

Peaks in DAKXUI01 that match CFZ—HCl or spleen CLDIs are italicized and marked with ^{~}.
Peaks in DAKXUI01 that match CFZ—TC are italicized and marked with ^{#}.
Peaks in DAKXUI01 that match CFZ—TC and CFZ—HCl or spleen CLDIs are italicized and marked with \hat{ }.
Peaks in DAKXUI01 that do not match any are only italicized.
Peaks in predicted pattern of CFZ—HCl that match CFZ—HCl or spleen CLDIs are in bold and marked with ^{~}.
Peaks in predicted pattern of CFZ—HCl that match CFZ—TC are in bold and marked with *..
Peaks in predicted pattern of CFZ—HCl that match CFZ—HCl or spleen CLDIs and CFZ—TC are in bold and marked with \hat{ }.
Peaks in predicted pattern of CFZ—HCl that do not match any are only bold
Peaks in either spleen CLDIs or CFZ—HCl that do not show up in the predicted pattern of CFZ—HCl are marked with an * alone.
Peaks in CFZ—HCl that do not match with spleen CLDIs are marked with a # alone

TABLE 5
Parameters of the crystal structure for CFZ—HCl (CCDC
deposition number: 1053818) compared to closest crystal analog
of neutral drug crystal CFZ—TC (CCDC refcode: DAKXUI01).

| | CFZ—HCl | DAKXUI01 |
|---|---|---|
| Formula | CFZ—HCl | CFZ |
| Formula Weight | 509.9 | 473.4 |
| Temperature (° C.) | −188 | 20 |
| Wavelength (Å) | 1.54178 | 0.7093 |
| Crystal System | Orthorhombic | Triclinic |
| Space Group | Pbca | P-1 |
| Color of Crystal | Dark Red | Orange-Red |
| No. of molecules/unit cell | 8 | 2 |
| a (Å) | 10.746 (14) | 10.507 (4) |
| b (Å) | 19.174 (4) | 12.852 (12) |
| c (Å) | 24.231 (3) | 9.601 (2) |
| α | 90 | 95.96 (4) |
| β | 90 | 97.22 (1) |
| γ | 90 | 69.73 (6) |
| V (Å³) | 4992.64 | 1204.01 |
| Density (calculated) (g/ml) | 1.357 | 1.306 |
| R | 0.176 | 0.062 |

TABLE 6
Peak Indexing for CFZ—HCl (2θ = 0-30°)-From Mercury.

| 2θ (°) | hkl |
|---|---|
| 7.3 | 002 |
| 9.1 | 020 |
| 9.5 | 110 |
| 10.0 | 111 |
| 11.5 | 022 |
| 14.0 | 122 |
| 14.5 | 004 |
| 16.2 | 200 |
| 16.9 | 210 |
| 17.6 | 211 |
| 18.2 | 202 |
| 18.9 | 220 |
| 20.2 | 222 |
| 20.4 | 141 |
| 21.0 | 142 |
| 21.5 | 230 |
| 21.9 | 231 |
| 22.2 | 125 |
| 22.7 | 232 |
| 23.2 | 050 |
| 23.5 | 106 |
| 24.0 | 116 |
| 24.5 | 150 |
| 25.2 | 215 |
| 25.8 | 007 |
| 27.1 | 243 |
| 27.2 | 027 |
| 28.3 | 061 |
| 28.7 | 251 |
| 29.0 | 244 |
| 30.2 | 063 |
| 30.5 | 324 |

TABLE 7
Peak Indexing for CFZ—TC (CCDC refcode-DAKXUI01)
(2θ = 0-30°)-From Mercury.

| 2θ (°) | hkl |
|---|---|
| 7.3 | 010 |
| 9.3 | 001 |
| 11.5 | 01(1) |
| 12.2 | 011 |
| 13.5 | 101 |
| 14.0 | 111 |
| 14.7 | 020 |
| 16.1 | 1(1)(1) |
| 16.8 | 02(1) |
| 18.0 | 200 |
| 18.2 | 21(1) |
| 18.6 | 002 |
| 19.9 | 10(2) |
| 21.9 | 13(1) |
| 23.2 | 2(1)(1) |
| 23.5 | 21(2) |
| 24.0 | 23(1) |
| 24.5 | 22(2) |
| 26.5 | 13(2) |
| 27.2 | 300 |
| 28.1 | 03(2) |
| 28.3 | 311 |
| 28.9 | 321 |
| 29.3 | 24(1) |

TABLE 8
pH of solutions and precipitate profile of clofazimine obtained
via addition of NH₄Cl at various concentrations.

| | | | | | NH₄Cl: |
|---|---|---|---|---|---|
| | | pH | | | CFZ |
| Reactive | | | Super- | Precipitate | molar |
| Agent | Clofazimine | Reactant | natant | Color | ratio |
| 1M NH₄Cl | 2 mM in MeOH | 4.89 | 6.07 | Dark Red | 500 |
| 0.8M NH₄Cl | 2 mM in MeOH | 4.95 | 6.08 | Dark Red | 400 |
| 0.6M NH₄Cl | 2 mM in MeOH | 4.95 | 6.00 | Dark Red | 300 |
| 0.4M NH₄Cl | 2 mM in MeOH | 5.14 | 6.30 | Dark Red | 200 |
| 0.2M NH₄Cl | 2 mM in MeOH | 5.69 | 6.63 | Dark Red | 100 |
| 0.1M NH₄Cl | 2 mM in MeOH | 6.04 | 6.77 | Dark Red | 50 |
| 0.08M NH₄Cl | 2 mM in MeOH | 6.08 | 6.90 | Orange | 40 |
| 0.06M NH₄Cl | 2 mM in MeOH | 6.18 | 7.26 | Orange | 30 |
| 0.04M NH₄Cl | 2 mM in MeOH | 6.24 | 7.09 | Orange | 20 |
| 0.02M NH₄Cl | 2 mM in MeOH | 6.41 | 7.44 | Orange | 10 |
| H₂O | 2 mM in MeOH | 8.02 | 9.4 | Orange | 0 |

### Example 5

### Drug Crystal Sequestration in Resident Macrophages Produces a Systemic Anti-Inflammatory Response

### Methods

Reagents

Anti-caspase 1 and anti-IL-1β antibodies were purchased from Thermo Pierce (Rockford, Ill.) and Novus Biologicals (Littleton, Colo.), respectively. Anti-actin antibody, LPS (from E. coli 055:B5) and carrageenan was purchased from Sigma (St. Louis, Mo.).

Mice Clofazimine Treatment

Clofazimine (CFZ, Sigma-Aldrich, C8895) was prepared in sesame oil (Shirakiku, Japan, or Roland, China) and Powdered Lab Diet 5001 (PMI International, Inc., St. Louis, Mo.) and orally administrated to Wild-type (WT) C57BL/6 mice or IL-1RA^{-/-} (4-5 week old, Jackson Laboratory, Bar Harbor, Me.) for up to 8 weeks ad libitum as previously described (Baik et al., 2013, supra; Baik et al., 2011, supra). Control mice were fed with the same diet without CFZ. The animal protocol was approved by the University of Michigan's Animal Care and Use Committee in accordance with the National Institutes of Health guidelines (UCUCA #PRO0005111).

Carrageenan Foot Edema Test

Foot inflammation in response to carrageenan injection was conducted in CFZ and control mice as previously described (Otterness I G, et al., Moore P F (1988) Immunochemical Techniques Part L: Chemotaxis and Inflammation (Elsevier) doi:10.1016/0076-6879(88)62086-6). In brief, the volume of each hind paw was measured with a caliper before and after the intraplantar injection of 30 μl 2% carrageenan (60 μg per paw) in PBS or equal volume of PBS in the contralateral paw. Paw swelling was measured at 4 and 48 h after injection, after which the animals were euthanized by exsanguination while deeply anesthetized with an intraperitoneal injection of ketamine (100 mg/kg) and xylazine (10 mg/kg), and the skin tissues of the plantar region were harvested for cytokine assay (see below).

Acute Lung Injury and Infrared Pulse Oximetry

Since C57BL/6 mice are relatively resistant towards a single dose of intratracheal (IT) LPS instillation (Matute-Bello G et al., 2008 Am J Physiol Lung Cell Mol Physiol 295(3):L379-99), two IT injections of LPS (16 mg/kg; 50 μl) were administered, one on day 0 and the second on day 3. Briefly, 8 wk CFZ-treated and control mice were anesthetized using intraperitoneal injections of xylazine (50 mg/kg) and ketamine (5 mg/kg). Under direct visualization of the vocal cords using an otoscope, either LPS or an equivalent volume of PBS in a 1 mL syringe attached to an oral gavage needle (22 G) was instilled into the lungs via the oral route. The mouse was then placed in a temperature-controlled cage (37° C.) for recovery from anesthesia.

The general health status of each PBS/LPS-instilled mice was monitored by measuring body weight and rectal temperature (Microprobe Thermometer, Physitemp Instruments, Clifton, N.J.), and cardiopulmonary function (arterial oxygen saturation, respiratory rate, heart rate and pulse distention) was monitored using MouseOx with a collar clip sensor (Starr Life Sciences Corp, Oakmont, Pa.) as previously described (Lax S et al., (2014) BMJ open Respir Res 1(1):e000014; Nayak S, et al. (2014) PLoS One 9(6):e98336). In brief, one day before IT instillations (D-1) the hair around the neck of each mouse was removed using Nair (Church & Dwight, Princeton, N.J.) to enable data acquisition using the collar clip sensor. The next day (D 0), immediately prior to the first LPS/PBS dose, and every 24 h afterwards until day 6 (D 6), the body weight, temperature and MouseOx readings were recorded. MouseOx data was acquired by very brief anesthesia of the mouse using 5% isoflurane to facilitate the placement of the collar clip sensor. The mouse was then placed in an enclosed chamber with ambient light and allowed to acclimatize for 5 min at which point the animals had recovered normal activities and physiological readings. Arterial oxygen saturation, breath rate, heart rate and pulse distention measurements were then simultaneously recorded for 6 min (15 readings/sec), and any errors caused by motion during recording were excluded, after which the mean value of each parameter was used for further data analysis.

Terminal Endpoint Assessment

To objectively assess LPS-induced mortality, a multi-parametric scoring system that relied on the daily changes in vital signs associated with inflammatory injury progression and mortality which included arterial oxygen saturation was used (Lax et al., 2014, supra), body weight and temperature (Toth L A (2000) ILAR J 41(2):72-79; Nemzek et al., (2004) Humane endpoints in shock research. Shock 21(1):17-25). First, the percent change from baseline (D 0) in arterial oxygen saturation, body weight, and temperature in each mouse caused by IT instillation of PBS/LPS were measured and calculated daily until day 6 post-instillation. These data were then used in a vector equation to calculate the distance between each LPS-treated mouse and the mean of the PBS-treated mice to assess the terminal endpoint for each LPS-instilled mouse (see Supplemental Information). CFZ-treated and control mice were calculated separately and LPS-instilled mice that scored a total of 18 or higher were determined as terminal, as these mice also displayed severe signs of sickness evidenced by impaired mobility, lack of grooming, hunched posture, and muscle weakness could be felt while handling the mice. These mice were immediately euthanized with ketamine/xylazine and the bronchoalveolar lavage (BAL) and lungs were harvested for cellular and biochemical analysis (see below). Remaining mice that did not reach terminal endpoint were all euthanized on day 6 post-PBS/LPS instillation.

Mouse Bronchoalveolar Lavage (BAL) Harvest and Isolation of Alveolar Macrophages, Immunohistochemistry and Imaging, SDS-PAGE and Western Blot, Cytokine Measurements

PBS/LPS-instilled CFZ- or control mice were euthanized by exsanguination while deeply anesthetized with an intraperitoneal injection of ketamine/xylazine. The trachea was surgically exposed and cannulated with an 18 G needle and the lungs were lavaged by instilling 1 ml DPBS (Life Technologies) containing 0.5 mM EDTA (Sigma). The retrieved BAL was then centrifuged (10 min at 400×g, 4° C.), and the supernatant was frozen (−80° C.), while the cell pellets were resuspended in 1 ml RPMI 1640 media (Life Technologies). To count total cells, the cells were stained with Trypan blue and counted using a hemocytometer. To distinguish cell types, an aliquot of the cells were dried on glass slides and stained with Diff Stain kit (IMEB Inc, San Marcos, Calif.) according to the manufacturer's instructions. To isolate alveolar macrophages, the cells were placed in 12-well culture plate (Corning, Tewksbury, Mass.) and washed with media after 45 min, enabling the isolation of alveolar macrophages by adherence.

Data Processing and Statistics

All data are expressed as mean±standard deviation (S.D.). For multiple comparisons, statistical analysis was performed with one-way analysis of variance (ANOVA) and Bonferroni's post-hoc comparisons. For two group comparisons an unpaired Student's t-test was used. All statistical analyses employed the Sigmaplot version 13 software and p≦0.05 was considered statistically significant.

### Results

Clofazimine Crystallization and Bioaccumulation in the Liver Occurs after 2 Weeks

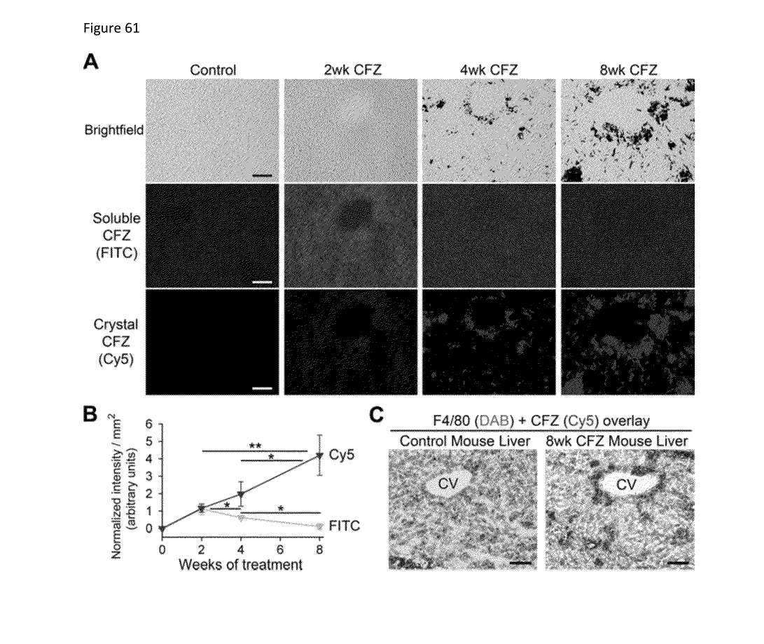

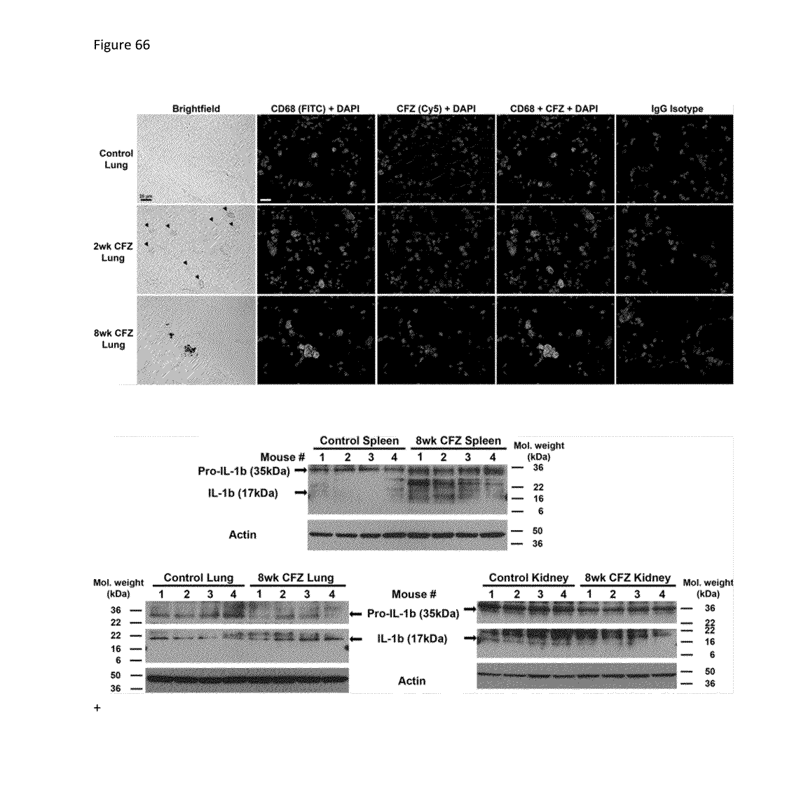

In the first two weeks of CFZ treatment, CFZ diffusely distributed throughout the liver (FIG. 61A, brightfield). However, by 4 weeks CFZ transformed into crystal form and microgranuloma structures can be seen. At 8 weeks, more CFZ crystals and larger microgranulomas are evident throughout the liver (FIG. 61A, top panel). Soluble CFZ is inherently fluorescent at FITC channel, but the protonation and chlorination of CFZ inside intracellular acidic compartments, as is the case with CFZ, causes a spectral shift to Cy5-positive and FITC-negative wavelengths (Baik et al., 2012, supra; Keswani R K et al., (2015) Cytometry A 87(9):855-67). Therefore, fluorescence microscopy enables in vivo detection of changes in the chemical composition of CFZ and the differentiation of soluble and crystal CFZ. This is illustrated by fluorescence microscopy of liver sections that, after 2 weeks of CFZ treatment, show high FITC fluorescence intensity that begins to diminish as Cy5 fluorescence intensity, and the formation of CFZ crystals, increases (FIG. 61A FITC and Cy5 panels and B). At 4 weeks, soluble CFZ concentration and the FITC signal continues to decline while the Cy5 signal and CFZ crystals become more evident (FIGS. 61A and B) such that by 8 weeks, the predominant signal is Cy5 (FIGS. 61A and B), indicating that CFZ exists almost entirely in crystal form. Immunohistochemical analysis of 8 week CFZ-treated livers shows that CFZ crystals are sequestered inside F4/80 positive macrophages, and a large population of CFZ crystal-containing macrophages are observed surrounding the central vein (FIG. 61C). This phenomenon similarly occurs in the lung as macrophages also contained numerous CFZ crystals at 8 weeks, but not at 2 weeks (FIG. 66). These data indicate that as the liver and lungs accumulate CFZ, CFZ is gradually protonated and converted into CFZ-HCl crystals in macrophages.

Clofazimine Bioaccumulation and Crystal Formation in the Liver Inhibits Inflammasome Activity and IL-1β Maturation while Enhancing IL-1RA Expression

The formation of insoluble drug precipitates or crystals inside cells is generally considered an adverse drug reaction, since intracellular crystals such as those formed by cholesterol or uric acid have been implicated in the activation of the NLRP3-caspase 1 inflammasome, which plays a pivotal role in the pathogenesis of chronic inflammatory disorders such as atherosclerosis (Duewell P, et al. (2010) Nature 464(7293):1357-61), non-alcoholic steatohepatitis (Ioannou G N et al., (2013) J Lipid Res 54(5):1326-34) and gout (Martinon F et al., (2006) Nature 440(7081):237-41). Uptake of other nano- and micro-particles has also been reported to cause inflammasome activation in macrophages (Simard J-C et al., (2015) J Biol Chem 290(9):5926-39; Demento S L, et al. (2009) Vaccine 27(23):3013-21; Peeters P M, et al. (2014) Part Fibre Toxicol 11:58). Yet, in spite of such concerns, there have not been any studies on the impact of CFZ crystal bioaccumulation on macrophage inflammasome proteins activity. Therefore, to understand whether soluble CFZ or the bioaccumulation of CFZ crystals alters the inflammasome, associated proteins in organs (liver, spleen and lung) known to form CFZ crystals and the kidneys were assessed, which do not, after 2 and 8 weeks of either CFZ or control treatment.

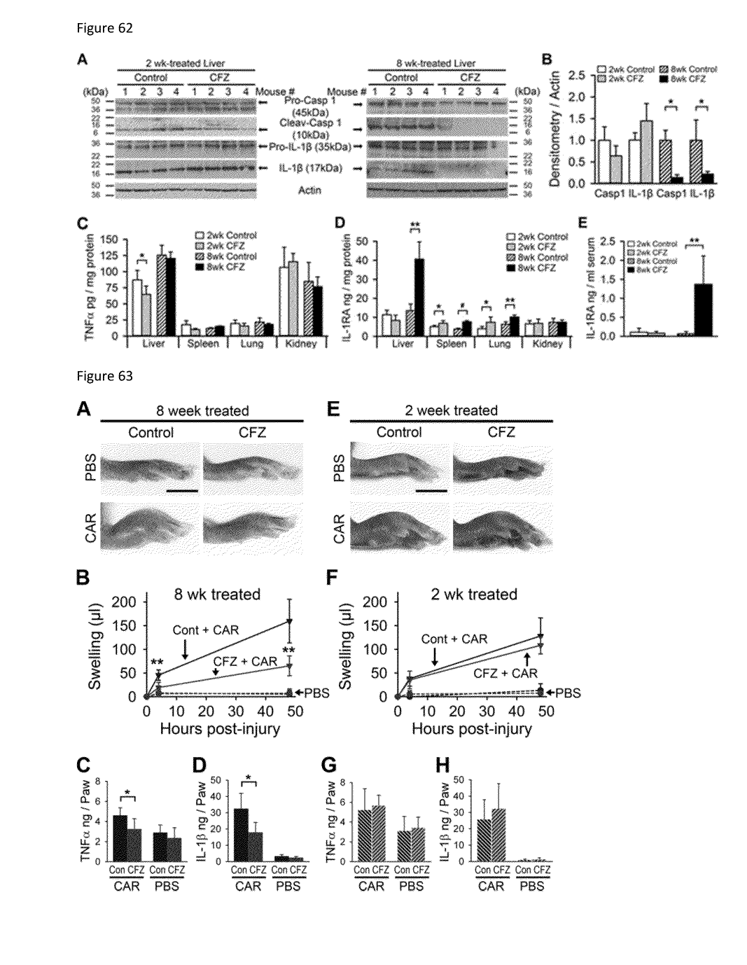

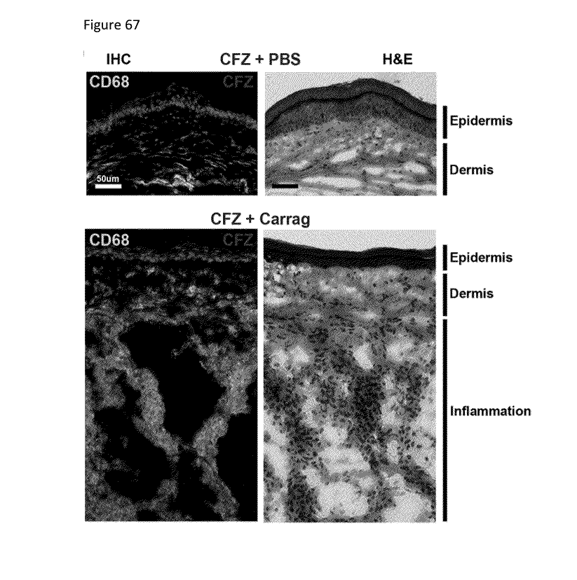

Two weeks of CFZ resulted in only moderate cleavage of hepatic caspase 1 and IL-1β (FIG. 62A left panel and B). However, by 8 weeks, caspase 1 cleavage and IL-β maturation was significantly reduced by CFZ treatment (FIG. 62A right panel and B); IL-1β levels in the spleen, lung and kidney did not change (FIG. 67). The expression of the pro-inflammatory cytokine, tumor-necrosis factor (TNF) α, was unchanged by CFZ at both 2 and 8 weeks (FIG. 62C) except in the liver where at 2 weeks it was increased.

Following previous observations that CFZ elevated the endogenous anti-inflammatory signaling molecule interleukin 1-receptor antagonist (IL-1RA) (Baik et al., 2013, supra), it was observed that CLDIs in liver, spleen and lung after 8 weeks of oral administration caused major upregulation of IL-1RA expression in these organs, especially in the liver, whereas IL-1RA levels in the kidney were unchanged (FIG. 62D). In contrast, following 2 weeks of oral drug administration, CFZ did not cause significant IL-1RA change in the liver, although the spleen and lung IL-1RA levels were slightly increased (FIG. 62D). As the liver is known to be a major source of circulating IL-1RA in blood (Gabay C et al., (1997) J Clin Invest 99(12):2930-40; Gabay C et al., (2001) Eur J Immunol 31(2):490-9), a corresponding increase in serum IL-1RA levels in CFZ-treated mice at 8 weeks, but not at 2 week was observed (FIG. 62E). Under all conditions, it was not possible to detect TNFα or IL-1β in the serum. In aggregate, these findings indicate that soluble CFZ minimally alters but that crystal CFZ decreases inflammasome-associated proteins in vivo. In parallel with this decline was an upregulation in the endogenous anti-inflammatory cytokine, IL-1RA.

### Systemic Clofazimine Bioaccumulation and Crystal Formation Dampens Carrageenan-Mediated Acute Footpad Inflammation

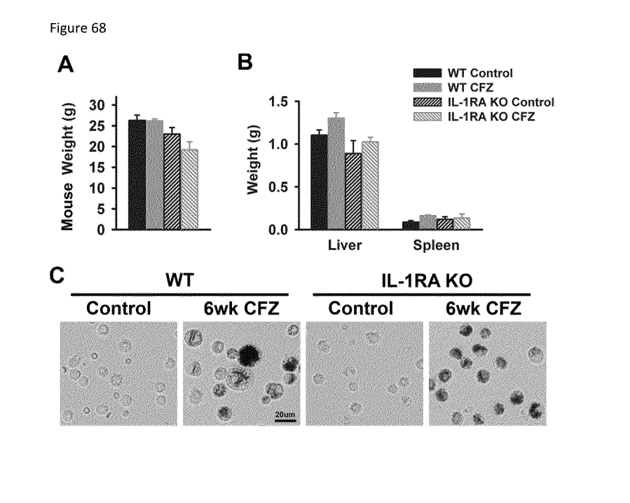

Since 8 wk CFZ-treated mice displayed high serum IL-1RA levels, the acute inflammatory response of CFZ-treated mice was tested by employing a well-established footpad injury model using an intraplantar injection of carrageenan. CFZ-treated and carrageenan-injected paws displayed strikingly reduced swelling 48 h post-injection compared to the paws of untreated mice (FIG. 63A). Paw volume measurements show that CFZ-treated mice exhibited a mean 57% (p=XX) reduction in paw swelling as early as 4 h post-injection compared to untreated mice and continued to exhibit reduced swelling over the 48 h study period (FIG. 63B). Carageenan-induced increases in footpad homogenate IL-1β and TNFα levels were also dramatically reduced by CFZ-treatment while IL-1RA levels were increased (FIG. 63C-E). Immunohistochemical analysis of footpad tissue sections, showed infiltration of macrophages at the site of injection but few CFZ crystals were visualized in the dermis of CFZ treated mice in both PBS- and carrageenan-injected footpads, indicating that the anti-inflammatory response was not dependent on the migration of CFZ crystal-containing macrophages into the site of tissue injury (FIG. 68).

To test whether the reduced inflammatory response observed in 8 wk CFZ treated mice was associated with CFZ crystal formation, carrageenan-induced footpad inflammation experiments were performed using mice that received 2 weeks of CFZ treatment (Baik et al., 2013, supra). At 48 h post-carageenan injection, only a modest reduction in paw swelling was observed compared with untreated mice (FIGS. 63F and G). The IL-1β and TNFα levels in footpad homogenates from these animals were similar in both CFZ- and control-treated mice at 48 h (FIGS. 63H and I). However, IL-1RA levels were reduced in the CFZ-treated mice (FIG. 97J).

### CFZ Dampening of the Acute Inflammatory Response in the Footpad is Mediated by Increased Circulating IL-1RA

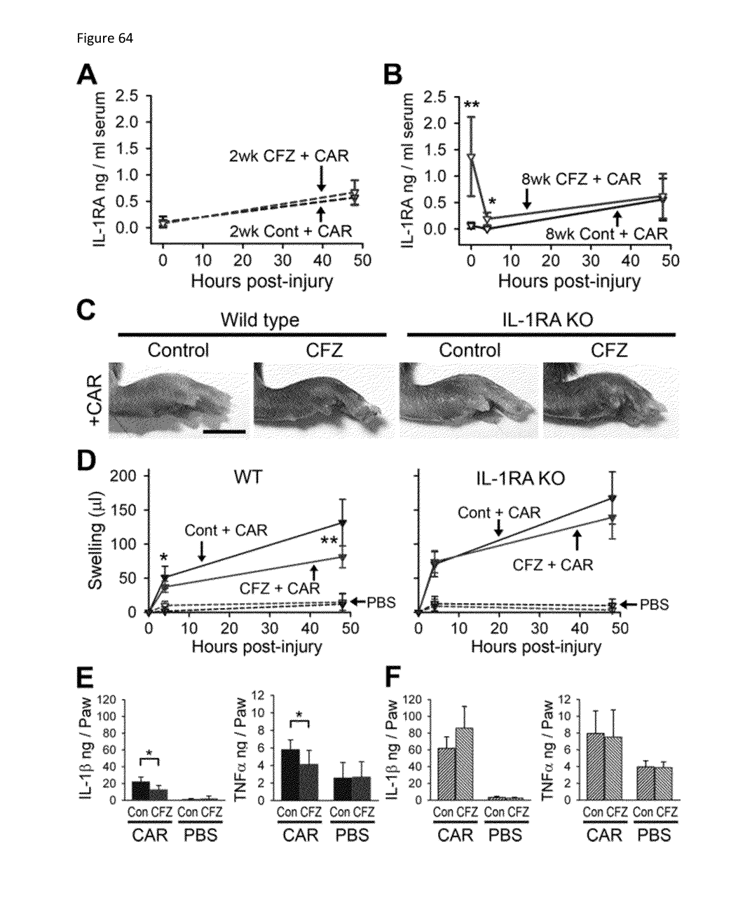

IL-1RA is known to be an early-acting acute-phase anti-inflammatory cytokine (Gabay et al., 1997, supra), and circulating IL-1RA has been reported to dampen a broad spectrum of inflammatory conditions (Dinarello C A, van der Meer J W M (2013) Semin Immunol 25(6):469-84). Its role is likely to inhibit IL-1β activity from spreading beyond a certain inflamed area while dampening the intensity of the inflammatory response (Arend W P (2002) Cytokine Growth Factor Rev 13(4-5):323-340). To see how serum IL-1RA levels changed following carrageenan injection into the paw, IL-1RA was detected in the serum of naïve (0 h), carrageenan-injected mice at 48 h and/or 4 h post-injection. Prior to the onset of CLDI formation, IL-1RA levels in the serum of control and 2 week CFZ treated mice were similar to those of uninjured mice. Comparing 2 week CFZ treated mice with untreated mice showed that IL-1RA increased to similar levels at 48 h post carrageenan injury (FIG. 64A). After massive biocrystal formation in 8 week CFZ-treated mice, plasma IL-1RA levels rapidly declined at 4 h post-injury, while in untreated control mice it remained constant (FIG. 64B). After 48 h, IL-1RA returned to similar levels in the plasma of control and 8 week CFZ-treated mice (FIG. 64B). For all mice, plasma levels of TNFα and IL-1β were below the detection limit of ELISAs (15 pg/ml). These results showed differences in circulating IL-1RA levels associated with differences in CFZ bioaccumulation in 2 vs 8 weeks CFZ treatment. In the 8 week treated mice, the increased levels of IL-RA circulating in plasma may be effective at dampening the carrageenan induced inflammatory response by binding and blocking IL-1 receptor signaling at the site of injury.

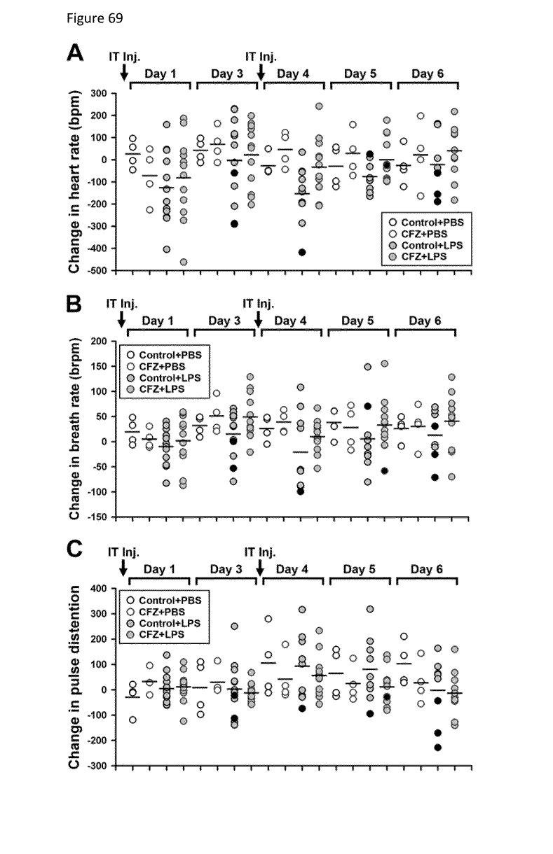

In order to further explore the possibility that elevated IL-1RA expression was mediating the anti-inflammatory activity of CFZ following CLDI formation, CFZ was given to age-matched IL-1RA KO and WT mice for 6 weeks, and tested using the footpad injury model. These experiments were done at 6 weeks and not at 8 weeks, because the IL-1RA KO mice weighed less than WT and their livers were smaller (FIG. 68A). Nevertheless, IL-1RA mice bioaccummulated CFZ comparably to WT mice (FIG. 68B). CLDI formation could be also seen in the IL-1RA KO alveolar macrophages (60% CLDI+ cells) at a similar level to WT (65% CLDI+ cells) (FIG. 69C). Remarkably, the anti-inflammatory effect of CFZ was greatly diminished in IL-1RA KO mice, as CFZ-treated and injured IL-1RA KO mice displayed similar swelling compared to untreated littermates (FIGS. 64C and D, right panels). In particular, the lack of anti-inflammatory activity was noticeable as early as 4 h post-injury as IL-1RA KO mice exhibit significant swelling at 4 h post-injury compared to similarly treated WT mice (FIG. 64D, left panel vs right panel). In CFZ-treated WT mice, reduced swelling compared to untreated and injured mice was observed (FIGS. 64C and D left panels), but the anti-inflammatory effects of CFZ were not anywhere near those observed in the control, 8 week CFZ treated WT mice (FIG. 63B). IL-1β levels were significantly reduced in WT CFZ-treated and injured mice, compared to untreated and injured mice (FIG. 64E, left panel). As expected, IL-1RA KO mice displayed radically increased IL-1β levels in both untreated and CFZ-treated mice with injury compared to WT mice (FIG. 64E, right panel). Carrageenan injury did not cause significant local TNFα expression in WT after 48 h, whereas in IL-1RA KO mice TNFα expression levels were significantly higher than controls (FIG. 64E, bottom panels). These results are consistent with CFZ bioaccumulation exerting systemic anti-inflammatory effects by elevating blood IL-1RA levels.

### CFZ Bioaccumulation Enhances Resistance to Acute Lung Injury and Improves Mouse Survival

Given the massive bioaccumulation of CFZ observed in vital organs such as the liver, spleen and lungs, it was determined whether the presence of CLDIs in these organs may sensitize the mice to a lethal pro-inflammatory injury. First, daily arterial oxygen saturation was measured as an indicator of lung function, as well as body weight, temperature, heart rate, breathing rate and pulse distention (measure of arterial blood flow, pulse pressure) to measure overall health. Baseline measurements of 8 wk CFZ-treated and control diet-treated mice show that 8 wk CFZ-treated mice weighed 6.7% less, and displayed lower breathing rate (10.7%), heart rate (9.3%) and pulse distention (16.9%) (Table 9). Arterial oxygen saturation was 1.5% higher in CFZ-treated than their control diet-treated counterparts (Table 9), indicating that lung function was normal or even enhanced by CFZ bioaccumulation. Body temperature was unaffected by CFZ treatment. Also, CFZ-treated mice did not display any behavioral signs of sickness as mobility, grooming, and posture were normal, and no signs of muscle weakness could be felt while handling the mice. These results indicate that although prolonged CFZ treatment and bioaccumulation caused certain changes in physiological parameters, these changes were not outside the ranges of normal physiological readings and seemed that CFZ bioaccumulation did not have any adverse effects on mouse overall health.

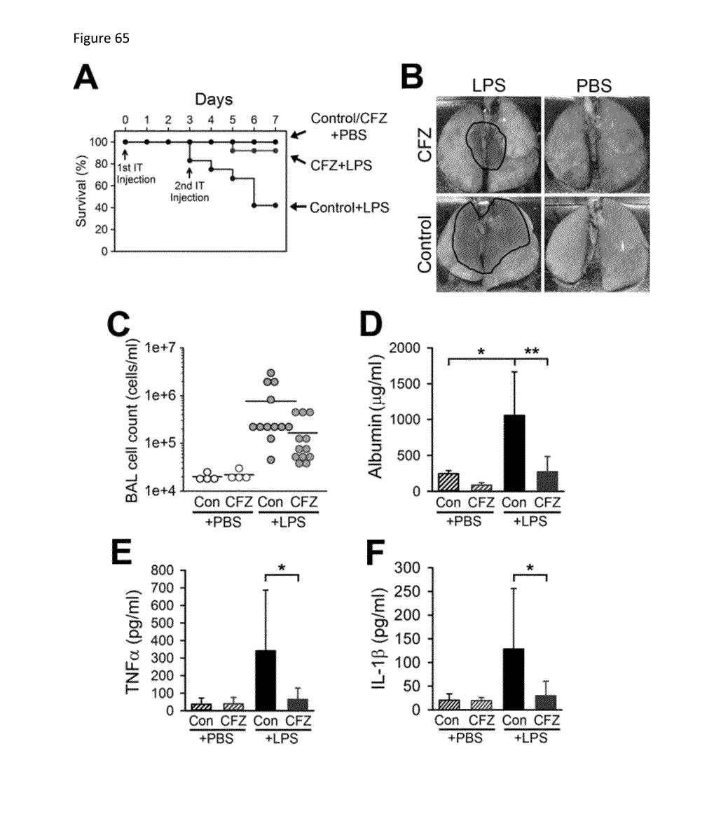

Next, the effect of a lethal dose of LPS on lung function and health of control diet-treated and CFZ-treated mice was evaluated. CFZ-treated mice were highly resistant to LPS-induced acute lung injury with a 92% survival rate compared to control diet-treated mice (42% survival rate) (FIG. 65A). The difference between control and CFZ-treated mice in arterial oxygen saturation levels was more prominent after the second LPS injection, and CFZ-treated mice displayed significantly higher arterial oxygen levels at day 5 and day 6 even though control mice that reached the endpoint on day 3 and 4 were euthanized, indicating that control mice lung function progressively deteriorated at a higher rate than CFZ-treated mice (FIG. 65B). Paralleling impaired lung function, untreated control mice injected with LPS dropped weight more rapidly compared to CFZ-treated mice, with 6 control mice losing more than 15% weight, compared to only 1 CFZ-treated mouse (FIG. 65C). Both control and CFZ-treated and LPS-injured mice displayed large drops in body temperature 1 day after LPS injection (days 1 and 4). However, CFZ-treated mice were more resistant to temperature drop after the second injection compared to control-mice (day 4) (FIG. 65D). CFZ-treated mice also displayed less reduction in heart rate, breathing rate and pulse distention after the second LPS injection (Supplemental FIGS. 65A, B and C) compared to control mice. Examination of lung injury after LPS instillation showed notably less lung injury in CFZ-treated mice compared to control mice as the area injured by LPS in the lung was marked reduced (FIG. 65E). In addition, CFZ mice displayed significantly less cell count in bronchioalveolar lavage (BAL) (FIG. 65F), as well as reduced TNFα and IL-1RA expression after LPS (FIGS. 65G and H). These data demonstrate that CFZ bioaccumulation as CLDIs in vital organs does not lead to decreased lung function and is associated with increase resistance towards acute injury via a lethal pro-inflammatory stimulus.

TABLE 9

| | Control diet | CFZ treated | |
|---|---|---|---|
| Vital signs | treated (n = 16) | (n = 16) | P value |
| Weight (g ± S.D) | 26.13 ± 1.49 | 24.39 ± 1.92 | 0.008 |
| Body Temperature | 35.30 ± 0.54 | 35.52 ± 0.76 | 0.35 |
| (° C. ± S.D) | | | |
| Arterial O₂ Saturation | 97.52 ± 0.76 | 99.01 ± 0.24 | 0.00000064 |
| (% ± S.D.) | | | |
| Breathing rate | 172.85 ± 18.25 | 154.29 ± 19.43 | 0.009 |
| (brpm ± S.D.) | | | |
| Heart rate | 632.91 ± 89.55 | 573.94 ± 85.28 | 0.066 |
| (bpm ± S.D.) | | | |
| Pulse distention | 512.22 ± 60.35 | 425.57 ± 52.32 | 0.00015 |
| (μm ± S.D) | | | |

All publications and patents mentioned in the present application are herein incorporated by reference. Various modification and variation of the described methods and compositions of the disclosure will be apparent to those skilled in the art without departing from the scope and spirit of the disclosure. Although the disclosure has been described in connection with specific preferred embodiments, it should be understood that the disclosure as claimed should not be unduly limited to such specific embodiments. Indeed, various modifications of the described modes for carrying out the disclosure that are obvious to those skilled in the relevant fields are intended to be within the scope of the following claims.

1. A composition comprising a biomimetic crystal of a pharmaceutical agent.

2. The composition of claim 1, wherein said biomimetic crystal is a pure drug crystal or in complex with a counterion or cell-stabilizing agent as a salt, hydrate, solvate or a cocrystal.

3. The composition of claim 1, wherein said biomimetic crystal has an orthorhombic, triclinic, or monoclinic crystal structure.

4. The composition of claim 1, wherein said crystal has a needle, cube, blade, prism, or rhomboid habit.

5. The composition of claim 1, wherein said small molecule pharmaceutical agent is clofazimine.

6. The composition of claim 3, wherein said orthorhombic crystalline structural form has the configuration space group Pbca.

7. The composition of claim 1, wherein the density of said crystals is between 1.25-1.4 g/ml.

8. The composition of claim 1, wherein said crystals have a size of 0.001-20 μm in each dimension.

9. The composition claim 1, wherein said counterion or cell stabilizing agent is hydrochloride.

10. The composition of claim 1, wherein said salt further comprises one or more additional ions.

11. The composition of claim 10, wherein said ions are selected from the group consisting of hydrochloride, hydrobromide, hydroiodide, hydrogen sulfate, sulfate, hydrogen phosphate, phosphate, carbonate, hydrogen carbonate, formate, gluconate, lactate, pyruvate, nitrite, glutarate, tartarate, benzoate, sulfate, fumarate, benzenesulfonate, tosylate, galacturonate, acetate, citrate, nitrate, oxalate, succinate, and maleate.

12. The composition of claim 1, wherein said composition further comprises a lipid.

13. The composition of claim 12, wherein said pharmaceutical agent is encapsulated by a liposome comprising said lipid.

14. The composition of claim 12, wherein said lipid is selected from the group consisting of phosphatidylcholine, cholesterol, phosphatidylethanolamine, phosphatidylglycerol, phosphatidylinositol, phosphatidylserine, sphingomyelin, cardiolipin, dioleoylphosphatidylglycerol (DOPG), diacylphosphatidylcholine, diacylphosphatidylethanolamine, ceramide, sphingomyelin, cephalin, cholesterol, cerebrosides, diacylglycerols, dioleoylphosphatidylcholine (DOPC), dimyristoylphosphatidylcholine (DMPC), and dioleoylphosphatidylserine (DOPS), diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), di stearoylphosphatidylcholine (DSPC), dipalmitoylphosphatidylcholine (DPPC), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide, polyethylene glycol (PEG), and PEG modified lipids.

15. A method of treating a disease in a subject, comprising:
administering the composition of claim 1 to a subject diagnosed with a disease.

16. The method of claim 15, wherein said disease is selected from the group consisting of asthma, bronchiolitis, bronchiolitis obliterans, chronic obstructive pulmonary disease (COPD), bronchitis, emphysema, hypersensitivity pneumonitis, idiopathic pulmonary fibrosis, pneumoconiosis, silicosis, meningitis, sepsis, malaria, rheumatoid osteoarthritis, psoriasis, acute respiratory disease syndrome, inflammatory bowel disease, multiple sclerosis, joint inflammation, reactive arthritis, hay fever, atherosclerosis, rheumatoid arthritis, bursitis, gouty arthritis, osteoarthritis, polymyalgia rheumatic arthritis, septic arthritis, infectious arthritis, asthma, autoimmune diseases, chronic inflammation, chronic prostatitis, glomerulonephritis, nephritis, inflammatory bowel diseases, pelvic inflammatory disease, reperfusion injury, transplant rejection, vasculitis, myocarditis, colitis, appendicitis, peptic ulcer, gastric ulcer, duodenal ulcer, peritonitis, pancreatitis, ulcerative colitis, seudomembranous colitis, acute colitis, ischemic colitis, diverticulitis, epiglottitis, achalasia, cholangitis, cholecystitits, hepatitis, Crohn's disease, enteritis, Whipple's disease, allergy, anaphylactic shock, immune complex disease, organ ischemia, reperfusion injury, organ necrosis, hay fever, sepsis, septicemia, endotoxic shock, cachexia, hyperpyrexia, eosinophilic granuloma, granulomatosis, sarcoidosis, septic abortion, epididymitis, vaginitis, prostatitis, urethritis, bronchitis, emphysema, rhinitis, pneumonitits, pneumoultramicroscopic silicovolcanoconiosis, alvealitis, bronchiolitis, pharyngitis, pleurisy, sinusitis, influenza, respiratory syncytial virus infection, HIV infection, hepatitis B virus infection, hepatitis C virus infection, herpes virus infection disseminated bacteremia, Dengue fever, candidiasis, malaria, filariasis, amebiasis, hydatidcysts, burns, dermatitis, dermatomyositis, sunburn, urticaria, Warts, Wheals, vasulitis, angiitis, endocarditis, arteritis, atherosclerosis, thrombophlebitis, pericarditis, myocarditis, myocardial ischemia, periarteritis nodosa, rheumatic fever, Alzheimer's disease, coeliac disease, congestive heart failure, adult respiratory distress syndrome, meningitis, encephalitis, multiple sclerosis, cerebral infarction, cerebral embolism, Guillame-Barre syndrome, neuritis, neuralgia, spinal cord injury, paralysis, uveitis, arthritides, arthralgias, osteomyelitis, fasciitis, Paget's disease, gout, periodontal disease, rheumatoid arthritis, synovitis, myasthenia gravis, thyroiditis, systemic lupus erythematosis, Goodpasture's syndrome, Behcet's syndrome, allograft rejection, graft-versus-host disease, Type I diabetes, Type II diabetes, ankylosing spondylitis, Berger's disease, Reiter's syndrome, Hodgkin's disease, ileus, hypertension, irritable bowel syndrome, myocardial infarction, sleeplessness, anxiety, local inflammation, and stent thrombosis.

17. The method of claim 16, wherein said disease is caused by infection by a microorganism selected from the group consisting of Staphylococcus aureus, Streptococcus, Streptococcus pneumonia, Neisseria gonorrhoeae, Mycobacterium tuberculosis, Borrelia burgdorferi, and Haemophilus influenza.

18. A method of identifying inflammation in a joint of a subject, comprising:
a) administering the composition claim 1 or clofazimine to said subject; and
b) performing photo-Acoustic Tomography (PAT) of said joint of said subject to identify the presence of said composition in said joint, wherein the presence of said composition in said joint is indicative of inflammation in said joint.

19. The method of claim 18, further comprising the step of imaging said joint using ultrasound.

20. The method of claim 18, wherein the presence of inflammation in said joint is indicative of arthritis in said joint.
